SEQUENCE LISTING (108 sequences)

SEQ ID NO: 1 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 2 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 3 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 4 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 5 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 6 - protein, 585 aa, Homo sapiens
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPR

SEQ ID NO: 7 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 8 - protein, 228 aa, Mus musculus
MGAWAMLYGVSMLCVLDLGQPSVVEEPGCGPGKVQNGSGNNTRCCSLYAPGKEDCPKERCICVTPEYHCGDPQCKICKHYPCQPGQRVESQGDIVFGFRCVACAMGTFSAGRDGHCRLWTNCSQFGFLTMFPGNKTHNAVCIPEPLPTEQYGHLTVIFLVMAACIFFLTTVQLGLHIWQLRRQHMCPRETQPFAEVQLSAEDACSFQFPEEERGEQTEEKCHLGGRWP

SEQ ID NO: 9 - protein, 124 aa, Artificial Sequence
  Description of Artificial Sequence Synthetic polypeptide
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 10 - protein, 107 aa, Artificial Sequence
  Description of Artificial Sequence Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 11 - protein, 123 aa, Artificial Sequence
  Description of Artificial Sequence Synthetic polypeptide
QVQLVESGGGVVQPGRSLRLSCAASGFTFSSYGFHWVRQAPGKGLEWVAVIWYAGSNKFYADSVKGRFTISRDNSKNTLYLQMNSLRAEDTAVYYCARGGQLDYYYYYVMDVWGQGTTVTVSS

SEQ ID NO: 12 - protein, 107 aa, Artificial Sequence
  Description of Artificial Sequence Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 13 - protein, 123 aa, Artificial Sequence
  Description of Artificial Sequence Synthetic polypeptide
QVQLVESGGGVVQPGRSLRLSCAASGFTFSSYGMHWVRQAPGKGLEWVAVIWYAGSNKYYADSVKGRFTISRDNSKNTLYLQMNSLRAEDTAVYYCARGGRIAVAFYYSMDVWGQGTTVTVSS

SEQ ID NO: 14 - protein, 107 aa, Artificial Sequence; identical to SEQ ID NO: 12
  Description of Artificial Sequence Synthetic polypeptide

SEQ ID NO: 15 - protein, 117 aa, Artificial Sequence
  Description of Artificial Sequence Synthetic polypeptide
QVQLQQWGAGLLKPSETLSLTCAVYGGSFSGYYWTWIRQPPGKGLEWIGKINHSGNTNYNPSLKSRVTISVDTSKNQFSLKLSSVTAADTAVYYCARLGAFDAFDIWGQGTMVTVSS

SEQ ID NO: 16 - protein, 107 aa, Artificial Sequence; identical to SEQ ID NO: 12
  Description of Artificial Sequence Synthetic polypeptide

SEQ ID NO: 17 - protein, 106 aa, Artificial Sequence
  Description of Artificial Sequence Synthetic polypeptide
EIVLTQSPATLSLSPGERATLSCRASQGVSSYLAWYQQKPGQAPRLLIYDASNRATGIPARFSGSGPGTDFTLTISSLEPEDFAVYYCQQRSNWHTFGQGTKLEIK

SEQ ID NO: 18 - protein, 124 aa, Artificial Sequence
  Description of Artificial Sequence Synthetic polypeptide
QVQLVESGGGVVQPGGSLRLSCAASGFILSDYGMHWVRQAPGKGLEWVTVIWYDGSNKFYVDSVKGRFTISRDNSKNTLYLQMNSLRVEDTAVYYCARGGRLATGHFYYVMDVWGQGTTVTVSS

SEQ ID NO: 19 - protein, 107 aa, Artificial Sequence; identical to SEQ ID NO: 12
  Description of Artificial Sequence Synthetic polypeptide

SEQ ID NO: 20 - protein, 124 aa, Artificial Sequence
  Description of Artificial Sequence Synthetic polypeptide
QVQLVESGGGVVQPGRSLRLSCTASGFTFSSYGMQWVRQAPGKGLEWVAVIWYEGSNKYYADSVKGRFTISRENSKNTLYLQMNSLRAEDTAVYYCARGGLMVRGLFYYGMDVWGQGTTVTVSS

SEQ ID NO: 21 - protein, 107 aa, Artificial Sequence
  Description of Artificial Sequence Synthetic polypeptide
AIQLTQSPSSLSASVGDRVTITCRASQGISSALAWYQQKPGKAPKFLIYDASSLESGVPSRFSGSGSGTDFTLTISSLQPEDFATYYCQQFNSYPYTFGQGTKLEIK

SEQ ID NO: 22 - protein, 125 aa, Artificial Sequence
  Description of Artificial Sequence Synthetic polypeptide
EVQLVESGGGLVKPGGSLRLSCAASGFTFSTVWMSWVRQAPGKGLEWVGRIKSKTDGGTTDYAAPVKGRFTISRDDSKNTLYLQMNSLHTEDTAVYYCTTGQLIPYSYYYGMDVWGQGTSVTVSS

SEQ ID NO: 23 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 24 - protein, 108 aa, Artificial Sequence
  Description of Artificial Sequence Synthetic polypeptide
EIVLTQSPGTLSLSPGERATLSCRASQSVTSSYLAWYQQKPGQAPRLLIYGASSRATGIPERFSGSGSGTDFTLTISRLEPEDFAVYYCQQYGSSPITFGQGTRLEIK

SEQ ID NO: 25 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEINHSGNTYYNPSLKSRVTISVDTSKNQLSLKLSSVTAADTAVYYCARFGSNDAFDIWGQGTMVTVSS

SEQ ID NO: 26 - protein, 107 aa, Artificial Sequence
  Description of Artificial Sequence Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKVEIK

SEQ ID NO: 27 - protein, 124 aa, Artificial Sequence
  Description of Artificial Sequence Synthetic polypeptide
QVQLVESGGGVVQPGRSLRLSCAASGFTFSNYGMHWVRQAPGKGLEWMAVIWYGGSNKFYADSVKGRFTISRDNSKNSLSLQMNSLRAEDTAVYYCARGGAMVRGVYYYGMDVWGQGTTVTVSS

SEQ ID NO: 28 - protein, 107 aa, Artificial Sequence
  Description of Artificial Sequence Synthetic polypeptide
AIQLTQSPSSLSASVGDRVTITCRASQGISSALAWYQQKPGKAPKFLIYDASSLESGVPSRFSGSGSGTDFTLTISSLQPEDFATYYCQQFNSYPQTFGQGTKVEIK

SEQ ID NO: 29 - protein, 107 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 30 - protein, 124 aa, Artificial Sequence
  Description of Artificial Sequence Synthetic polypeptide
QVQLVESGGDVVQPGRSLRLSCAASGFTFSTYGMHWVRQAPGKGLEWVAVTWYAGSNKFYADSVKGRFTISRDNSKNTLYLQMNSLRAEDTAVYYCARGGSMVRGLYYYGMDVWGQGTTVTVSS

SEQ ID NO: 31 - protein, 107 aa, Artificial Sequence; identical to SEQ ID NO: 10
  Description of Artificial Sequence Synthetic polypeptide

SEQ ID NO: 32 - protein, 118 aa, Artificial Sequence
  Description of Artificial Sequence Synthetic polypeptide
QVQLVQSGAEVKKPGASVKVSCKGSGYTFTDYAMYWVRQAPGQGLEWIGVIRTYSGDVTYNQKFKDRATMTVDKSISTAYMELSRLRSDDTAVYYCAKSGTVRGFAYWGQGTLVTVSS

SEQ ID NO: 33 - protein, 113 aa, Artificial Sequence
  Description of Artificial Sequence Synthetic polypeptide
DIVMTQSPPTLSLSPGERVTLSCKSSQSLLNSGNQKNYLTWYQQKPGQAPRLLIYWASTRESGIPARFSGSGSGTDFTLTISSLQPEDFAVYHCQNDYSYPYTFGQGTKLEIK

SEQ ID NO: 34 - protein, 113 aa, Artificial Sequence
  Description of Artificial Sequence Synthetic polypeptide
DIVMTQSPDSLAVSLGERATINCKSSQSLLNSGNQKNYLTWYQQKPGQPPKLLIYWASTRESGVPDRFSGSGSGTDFTLTISSLQAEDVAVYHCQNDYSYPYTFGQGTKLEIK

SEQ ID NO: 35 - protein, 118 aa, Artificial Sequence
  Description of Artificial Sequence Synthetic polypeptide
QVQLVQSGAEVKKPGASVKVSCKASGYTFTGYAMYWVRQAPGQGMEWIGVIRTFSGDVTYNQKFRGRATMTVDTSISTAYMELSRLRSDDTAVYYCAKSGTVRGFAYWGQGTLVTVSS

SEQ ID NO: 36 - protein, 113 aa, Artificial Sequence
  Description of Artificial Sequence Synthetic polypeptide
DIVMTQSPDSLAVSLGERATINCKSSQSLLNSGNQKNYLSWYHQKPGQPPKMLIYWASTRESGVPDRFSGSGSGTDFTLTISSVQAEDVAVYHCQNDHSFPYTFGQGTKLEIK

SEQ ID NO: 37 - protein, 123 aa, Artificial Sequence
  Description of Artificial Sequence Synthetic polypeptide
QVQVVESGGGVVQPGRSLRLSCAASGFTFSSYGMHWVRQAPGKGLEWVSVIWYEGSNKYYADSVKGRFTISRDNSKNTLYLQMNSLRAEDTAVYYCARGGLLGYYYYYGMDVWGQGTTVTVSS

SEQ ID NO: 38 - protein, 108 aa, Artificial Sequence
  Description of Artificial Sequence Synthetic polypeptide
DIQMTQSPSSLSASVGDRVTITCRASQGIRNDLGWYQQKPGKAPKRLIYDASSLQSGVPSRFSGSGSGTEFTLTISSLQPEDFATYYCLQHHSYPWTFGQGTKVEIKR

SEQ ID NO: 39 - protein, 123 aa, Artificial Sequence
  Description of Artificial Sequence Synthetic polypeptide
QVQLVESGGGVVQPGRSLRLSCAASGFTFSSYGMHWVRQAPGKGLEWVAVIWYPGSNKYYADSVKGRFTISRDNSKNTLYLQMNSLRAEDTAVYYCARGGELGRYYYYGMDVWGQGTTVTVSS

SEQ ID NO: 40 - protein, 108 aa, Artificial Sequence
  Description of Artificial Sequence Synthetic polypeptide
DIQMTQSPSSLSASVGDRVTVTCRASQGIRNDLGWYQQKPGKAPKRLIYAASSLQSGVPSRFSGSGSGTEFTLTISSLQPEDFATYYCLQHNNYPWTFGQGTKVDIKR

SEQ ID NO: 41 - protein, 125 aa, Artificial Sequence
  Description of Artificial Sequence Synthetic polypeptide
QVQLQESGPGLVKPSQTLSLTCTVSGGSISSGGYFWSWIRQHPGKGLEWIGYIYYSGTTYYNPSLKSRVTISIDTSKNHFSLKLSSVTAADTAVYYCARDLFYYDSSGPRGFDPWGQGTLVTVSS

SEQ ID NO: 42 - protein, 109 aa, Artificial Sequence
  Description of Artificial Sequence Synthetic polypeptide
EIVLTQSPGTLSLSPGERATLSCRASQTVSSNYLAWYQQKPGQAPRLLIYGSSTRATGIPDRFSGSGSGTDFTLTISRLEPEDFAVYYCQQYDSSPWTFGQGTKVEIKR

SEQ ID NO: 43 - protein, 123 aa, Artificial Sequence
  Description of Artificial Sequence Synthetic polypeptide
QVQLVESGGGVVQPGRSLRLSCAASGFTFSSYGMHWVRQAPGKGLEWMAVIWYVGSNKYYADSVKGRFTISRDNSKNTLYLQMNSLSAEDTAVYYCARGGELGRDYYSGMDVWGQGTTVTVSS

SEQ ID NO: 44 - protein, 108 aa, Artificial Sequence
  Description of Artificial Sequence Synthetic polypeptide
DIQMTQSPSSLSASVGDRVTITCRASQGIRNDLGWYQQKPGKAPKRLIYAASSLQSGVPSRFSGSGSGTEFTLTISSLQPEDFATYYCQQHNSYPWTFGQGTKVEIKR

SEQ ID NO: 45 - protein, 123 aa, Artificial Sequence
  Description of Artificial Sequence Synthetic polypeptide
QVQLVESGGGVVQPGRSLRLSCVASGFTFSSYGMHWIRQAPGKGLEWVAVIWYEGSNKYYADSVKGRFTISRDNSKNTLYLQMNSLRAEDTAVYYCARGGRLGKDYYSGMDVWGQGTTVTVSS

SEQ ID NO: 46 - protein, 108 aa, Artificial Sequence
  Description of Artificial Sequence Synthetic polypeptide
DIQMTQSPSSLSASVGDRVTITCRASQGIRNDLGWYQQKPGKAPNRLIYATSSLQSGVPSRFSGSGSGTEFTLTISSLQPEDFATYYCLQHNTYPWTFGQGTKVEIKR

SEQ ID NO: 47 - protein, 125 aa, Artificial Sequence
  Description of Artificial Sequence Synthetic polypeptide
QVQLQESGPGLVKPSETLSLTCTVSGGSISSGGYFWSWIRQPPGKGLEWIGYIYYSGTTYYNPSLKSRVTISIDTSKNQFSLKLSSVTAADTAVYYCARDLFYYDTSGPRGFDPWGQGTLVTVSS

SEQ ID NO: 48 - protein, 109 aa, Artificial Sequence; identical to SEQ ID NO: 42
  Description of Artificial Sequence Synthetic polypeptide

SEQ ID NO: 49 - protein, 123 aa, Artificial Sequence
  Description of Artificial Sequence Synthetic polypeptide
QVQLVESGGGVVQPGRSLRLSCAASGFTFSSYGMHWVRQAPGKGLEWMAVIWYVGSNKYYADSVKGRFTISRDNSKNTLYLQMNSLRAEDTAVYYCARGGELGRDYYSGMDVWGQGTTVTVSS

SEQ ID NO: 50 - protein, 108 aa, Artificial Sequence; identical to SEQ ID NO: 44
  Description of Artificial Sequence Synthetic polypeptide

SEQ ID NO: 51 - protein, 123 aa, Artificial Sequence
  Description of Artificial Sequence Synthetic polypeptide
QVQLVESGGGVVQPGRSLRLSCVASGFTFSSYGMHWIRQAPGKGLEWVAVIWYEGSNKYYAESVKGRFTISRDNSKNTLYLQMNSLRAEDTAVYYCARGGRLGKDYYSGMDVWGQGTTVTVSS

SEQ ID NO: 52 - protein, 108 aa, Artificial Sequence
  Description of Artificial Sequence Synthetic polypeptide
DIQMTQSPSSLSASVGDRVTITCRASQGIRNDLGWYQQKPGKAPKRLIYATSSLQSGVPSRFSGSGSGTEFTLTISSLQPEDFATYYCLQHNTYPWTFGQGTKVEIKR

SEQ ID NO: 53 - protein, 123 aa, Artificial Sequence
  Description of Artificial Sequence Synthetic polypeptide
QVQVVESGGGVVQPGRSLRLSCAASGFTFSSYGMHWVRQAPGKGLEWVSVIWYEGSNKYYAESVKGRFTISRDNSKNTLYLQMNSLRAEDTAVYYCARGGLLGYYYYYGMDVWGQGTTVTVSS

SEQ ID NO: 54 - protein, 108 aa, Artificial Sequence; identical to SEQ ID NO: 38
  Description of Artificial Sequence Synthetic polypeptide

SEQ ID NO: 55 - protein, 123 aa, Artificial Sequence
  Description of Artificial Sequence Synthetic polypeptide
QVQLVESGGGVVQPGRSLRLSCAASGFTFSSYGMHWVRQAPGKGLEWVAVIWYPGSNKYYAESVKGRFTISRDNSKNTLYLQMNSLRAEDTAVYYCARGGELGRYYYYGMDVWGQGTTVTVSS

SEQ ID NO: 56 - protein, 108 aa, Artificial Sequence; identical to SEQ ID NO: 40
  Description of Artificial Sequence Synthetic polypeptide

SEQ ID NO: 57 - protein, 130 aa, Artificial Sequence
  Description of Artificial Sequence Synthetic polypeptide
EVQLVQSGGGLVQPGGSLRLSCSASGFSFSSYAMHWVRQAPGKGLEYVSGISDNGGSTKYADSVKGRFTISRDNSQNTLYLQMSSLRSEDTAVYYCARGGPTYYDFWSGYYTDEDAFDIWGQGTLVTVSS

SEQ ID NO: 58 - protein, 107 aa, Artificial Sequence
  Description of Artificial Sequence Synthetic polypeptide
EIVMTQSPSSLSASVGDRVTITCRASQSINNYLNWYQQKPGKAPKLLIYATSRLQSGVPSRFSGSGSGADLTLTISSLQPEDVATYYCQQSYSFPWTFGQGTKVEIK

SEQ ID NO: 59 - protein, 134 aa, Artificial Sequence
  Description of Artificial Sequence Synthetic polypeptide
EVQLEVQLVQSGGGLVQPGGSLRLSCSASGFSFSSYAMHWVRQAPGKGLEYVSGISDNGGSTKYADSVKGRFTISRDNSQNTLYLQMSSLRSEDTAVYYCARGGPTYYDFWSGYYTDEDAFDIWGQGTLVTVSS

SEQ ID NO: 60 - protein, 107 aa, Artificial Sequence; identical to SEQ ID NO: 58
  Description of Artificial Sequence Synthetic polypeptide

SEQ ID NO: 61 - protein, 128 aa, Artificial Sequence
  Description of Artificial Sequence Synthetic polypeptide
QVQLVQSGTQVKMPGASVKVSCKASGYTFDDYGIGWVRQAPGQGLEWMGWISPYTHRTNSSPKLQDRVTMTTDTSTSTAYMELRSLRSDDTAVYYCARDGTYYDFWSGYFDNGAFDIWGQGTLVTVSS

SEQ ID NO: 62 - protein, 110 aa, Artificial Sequence
  Description of Artificial Sequence Synthetic polypeptide
QSVVTQPPSVSAAPGQKVTISCSGSTSNIGNNYVSWYQQLPGTAPKLLIYDNYKRPSGIPDRFSGSKSGTSATLGITGLRTGDEADYFCGTWDSSLNAWVFGGGTKLTVL

SEQ ID NO: 63 - protein, 123 aa, Artificial Sequence
  Description of Artificial Sequence Synthetic polypeptide
EVQLLESGGGLIQPGGSLRLSCAASGFTFSTYGMSWVRQAPGKGLEWVSGITGSAGGGSTNYADSVKGRFTISRDNSKNTLYLQMNSLRAEDTAVYYCAKGYSSNWRSAFDIWGQGTMVTVSS

SEQ ID NO: 64 - protein, 108 aa, Artificial Sequence
  Description of Artificial Sequence Synthetic polypeptide
SYELMQPPSVSVSPGQTAGITCSGDALPKQYAYWYQQRPGQAPVLLIYKDTERPSGIPERFSGSSSGTTVTLTISGVQAEDEADYYCQSADSSGTYPVFGGGTKLTVL

SEQ ID NO: 65 - protein, 127 aa, Artificial Sequence
  Description of Artificial Sequence Synthetic polypeptide
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGIAAAGPPYYYYYYYMDVWGKGTTVTVSS

SEQ ID NO: 66 - protein, 107 aa, Artificial Sequence
  Description of Artificial Sequence Synthetic polypeptide
DIQMTQSPSSLSASVGDRVTITCRASQTIYNYLNWYQQKPGKAPKLLIYAASSLQSGVPSRFGGRGYGTDFTLTINSLQPEDFATYFCQQSYTSPLTFGQGTKVDIK

SEQ ID NO: 67 - protein, 115 aa, Artificial Sequence
  Description of Artificial Sequence Synthetic polypeptide
QVTLVESGGGLVKPGGSLTLSCGASGFTISSYAMSWVRQSPGKALEWVAIISTGGSTYYPDSVRGRFTISRDNAKNSLYLTMSSLDSVDTAMYYCARVGGYYDSMDHWGQGTSVT

SEQ ID NO: 68 - protein, 109 aa, Artificial Sequence
  Description of Artificial Sequence Synthetic polypeptide
DIVLTQSPASLAASVGDRATISCRASETVDNYGISFMNWFQQKPGKSPKLLIYAASNQGSGVPARFSGSGSGTDFSLNIHPMQPDDTATYFCQQSKEVPWTFGGGTKLE

SEQ ID NO: 69 - protein, 115 aa, Artificial Sequence
  Description of Artificial Sequence Synthetic polypeptide
QVTLVESGGGLVKPGGSLTLSCGASGFTISSYAMSWVRQSPGKALEWVAIISTGGSTYYPDSVRGRFTISRDNAKNSLYLTMSSLDSVDTATYYCARVGGYYDSMDHWGQGTSVT

SEQ ID NO: 70 - protein, 115 aa, Artificial Sequence
  Description of Artificial Sequence Synthetic polypeptide
QVTLVESGGGLVKPGGSLTLSCGASGFTISSYAMSWVRQSPGKALEWVAIISTGGSTYYPDKFRGRFTISRDNAKNSLYLTMSSLRSEDTATYYCARVGGYYDSMDHWGQGTSVT

SEQ ID NO: 71 - protein, 109 aa, Artificial Sequence
  Description of Artificial Sequence Synthetic polypeptide
DIVLTQSPASLSASVGDRATISCRASETVDNYGISFMNWFQQKPGQSPKLLIYAASNQGSGVPARFSGSGSGTDFSLTISPMQPDDTATYYCQQSKEVPWTFGGGTKLE

SEQ ID NO: 72 - protein, 115 aa, Artificial Sequence
  Description of Artificial Sequence Synthetic polypeptide
QVTLKESGGGLVKPGGSLTLSCGASGFTISSYAMSWVRQSPGKALEWVAIISTGGSTYYPDKFRGRFTISRDNAKNSLYLTMSSLRSEDTATYYCARVGGYYDSMDHWGQGTSVT

SEQ ID NO: 73 - protein, 115 aa, Artificial Sequence
  Description of Artificial Sequence Synthetic polypeptide
EVQLVESGGGLIQPGGSLKLSCAASGFTISSYAMSWVRQAPGKGLEWVAIISTGGSTYYADSVKGRFTISRDNSKNTLYLQMNSLRAEDTAVYYCARVGGYYDSMDHWGQGTSVT

SEQ ID NO: 74 - protein, 109 aa, Artificial Sequence
  Description of Artificial Sequence Synthetic polypeptide
DIVLTQSPASLAVSPGQRATITCRASETVDNYGISFMNWFQQKPGQPPKLLIYAASNQGSGVPARFSGSGSGTDFTLTINPVEADDTANYYCQQSKEVPWTFGQGTKVE

SEQ ID NO: 75 - protein, 122 aa, Artificial Sequence
  Description of Artificial Sequence Synthetic polypeptide
  MOD_RES (24)..(24): Any amino acid
  MOD_RES (69)..(69): Any amino acid
  MOD_RES (73)..(73): Any amino acid
  MOD_RES (75)..(75): Any amino acid
  MOD_RES (80)..(80): Any amino acid
  MOD_RES (98)..(98): Any amino acid
QVQLVESGGGVVQPGRSLRLSCAXSGFSLSTSGMGVGWVRQAPGKGLEWVAHIWWDDDKYYSPSLKSRXTISXDXSKNTXYLQMNSLRAEDTAVYYCXRSYYYGSSGAMDYWGQGTLVTVSS

SEQ ID NO: 76 - protein, 113 aa, Artificial Sequence
  Description of Artificial Sequence Synthetic polypeptide
DIVMTQSPLSLPVTPGEPASISCRSSQSLVHSDGNTYLHWYLQKPGQSPQLLIYKVSKRFSGVPDRFSGSGSGTDFTLKISRVEAEDVGVYYCSQSTHVPPTFGQGTKVEIKR

SEQ ID NO: 77 - protein, 123 aa, Artificial Sequence
  Description of Artificial Sequence Synthetic polypeptide
  MOD_RES (97)..(98): Any amino acid
QVQLVESGGGVVQPGRSLRLSCAASGFTFSDYYMAWVRQAPGKGLEWVAYIHANGGSTYYRDSVRGRFTISRDNSKNTLYLQMNSLRAEDTAVYYCXXGSFMYAADYYIMDAWGQGTLVTVSS

SEQ ID NO: 78 - protein, 113 aa, Artificial Sequence
  Description of Artificial Sequence Synthetic polypeptide
DIVMTQSPLSLPVTPGEPASISCRSSQSLLHSDGNTFLSWYLQKPGQSPQLLIYLASNRFSGVPDRFSGSGSGTDFTLKISRVEAEDVGVYYCFQHTHLPLTFGQGTKVEIKR

SEQ ID NO: 79 - protein, 118 aa, Artificial Sequence
  Description of Artificial Sequence Synthetic polypeptide
  MOD_RES (48)..(48): Any amino acid
  MOD_RES (68)..(68): Any amino acid
  MOD_RES (70)..(70): Any amino acid
  MOD_RES (72)..(72): Any amino acid
QVQLVQSGAEVKKPGASVKVSCKASGYTFSRYWIEWVRQAPGQGLEWXGEILPGSGSSNYNEKFKDRXTXTXDTSTSTAYMELRSLRSDDTAVYYCARKVYYYAMDFWGQGTLVTVSS

SEQ ID NO: 80 - protein, 109 aa, Artificial Sequence
  Description of Artificial Sequence Synthetic polypeptide
  MOD_RES (48)..(48): Any amino acid
  MOD_RES (72)..(72): Any amino acid
EIVLTQSPGTLSLSPGERATLSCTASSSVSSSYFHWYQQKPGQAPRLXIYSTSNLASGIPDRFSGSGSGTDXTLTISRLEPEDFAVYYCHQYHRSPRTFGQGTKVEIKR

SEQ ID NO: 81 - protein, 121 aa, Artificial Sequence
  Description of Artificial Sequence Synthetic polypeptide
  MOD_RES (24)..(24): Any amino acid
  MOD_RES (50)..(51): Any amino acid
  MOD_RES (69)..(69): Any amino acid
  MOD_RES (71)..(71): Any amino acid
  MOD_RES (73)..(73): Any amino acid
  MOD_RES (80)..(80): Any amino acid
  MOD_RES (99)..(99): Any amino acid
QVQLQESGPGLVKPSETLSLTCTXSGFSLSTYGVGVGWIRQPPGKGLEWXXNIWWDDDNYYNPSLIHRXTXSXDTSKNQXSLKLSSVTAADTAVYYCAXIKEPRDWFFEFWGQGTLVTVSS

SEQ ID NO: 82 - protein, 108 aa, Artificial Sequence
  Description of Artificial Sequence Synthetic polypeptide
  MOD_RES (46)..(46): Any amino acid
  MOD_RES (49)..(49): Any amino acid
  MOD_RES (71)..(71): Any amino acid
DIQMTQSPSSLSASVGDRVTITCRASQGVNNFLTWYQQKPGKAPKXLIXYTSNLQSGVPSRFSGSGSGTDXTLTISSLQPEDFATYYCQQYHGFPNTFGQGTKVEIKR

SEQ ID NO: 83 - protein, 121 aa, Artificial Sequence
  Description of Artificial Sequence Synthetic polypeptide
  MOD_RES (24)..(24): Any amino acid
  MOD_RES (50)..(51): Any amino acid
  MOD_RES (69)..(69): Any amino acid
  MOD_RES (73)..(73): Any amino acid
  MOD_RES (80)..(80): Any amino acid
  MOD_RES (99)..(99): Any amino acid
QVQLQESGPGLVKPSETLSLTCTXSGFSLSTYGVGVGWIRQPPGKGLEWXXNIWWDDDKYYNPSLKNRXTISXDTSKNQXSLKLSSVTAADTAVYYCAXIKEPRDWFFEFWGQGTLVTVSS

SEQ ID NO: 84 - protein, 108 aa, Artificial Sequence
  Description of Artificial Sequence Synthetic polypeptide
  MOD_RES (46)..(46): Any amino acid
  MOD_RES (49)..(49): Any amino acid
  MOD_RES (71)..(71): Any amino acid
DIQMTQSPSSLSASVGDRVTITCRASQGVNNYLTWYQQKPGKAPKXLIXYTSNLQSGVPSRFSGSGSGTDXTLTISSLQPEDFATYYCQQYHGFPNTFGQGTKVEIKR

SEQ ID NO: 85 - protein, 117 aa, Artificial Sequence
  Description of Artificial Sequence Synthetic polypeptide
  MOD_RES (96)..(96): Any amino acid
QVQLVESGGGVVQPGRSLRLSCAASGFTVRNYAMSWVRQAPGKGLEWVASISTGDRSYLPDSMKGRFTISRDNSKNTLYLQMNSLRAEDTAVYYCXRYFDFDSFAFWGQGTLVTVSS

SEQ ID NO: 86 - protein, 108 aa, Artificial Sequence
  Description of Artificial Sequence Synthetic polypeptide
DIQMTQSPSSLSASVGDRVTITCRASQDINNFLNWYQQKPGKAPKLLIYYTSKLHSGVPSRFSGSGSGTDFTLTISSLQPEDFATYYCQQGHTLPPTFGQGTKVEIKR

SEQ ID NO: 87 - protein, 120 aa, Artificial Sequence
  Description of Artificial Sequence Synthetic polypeptide
  MOD_RES (47)..(48): Any amino acid
  MOD_RES (71)..(71): Any amino acid
  MOD_RES (96)..(96): Any amino acid
QVQLQESGPGLVKPSETLSLTCTVSGDSITSGYWNWIRQPPGKGLEXXGYISYSGSTYYNPSLRGRVTISXDTSKNQFSLKLSSVTAADTAVYYCXRRHLGSGYGWFAYWGQGTLVTVSS

SEQ ID NO: 88 - protein, 109 aa, Artificial Sequence
  Description of Artificial Sequence Synthetic polypeptide
  MOD_RES (71)..(71): Any amino acid
DIVMTQSPDSLAVSLGERATINCKASQDVNTAVAWYQQKPGQPPKLLIYWASTRHTGVPDRFSGSGSGTDXTLTISSLQAEDVAVYYCQQHSYTPPWTFGQGTKVEIKR

SEQ ID NO: 89 - protein, 118 aa, Artificial Sequence
  Description of Artificial Sequence Synthetic polypeptide
QVQLVESGGGVVQPGRSLRLSCAASGFTFSSYAMSWVRQAPGKGLEWVASISSGGTTYYPDSVKGRFTISRDNSKNTLYLQMNSLRAEDTAVYYCARVGGYYDSMDYWGQGTLVTVSS

SEQ ID NO: 90 - protein, 112 aa, Artificial Sequence
  Description of Artificial Sequence Synthetic polypeptide
  MOD_RES (31)..(31): Any amino acid
  MOD_RES (57)..(57): Any amino acid
EIVLTQSPGTLSLSPGERATLSCRASESVDXYGVSFMNWYQQKPGQAPRLLIYAASXQGSGIPDRFSGSGSGTDFTLTISRLEPEDFAVYYCQQTKEVTWTFGQGTKVEIKR

SEQ ID NO: 91 - protein, 120 aa, Artificial Sequence
  Description of Artificial Sequence Synthetic polypeptide
  MOD_RES (47)..(48): Any amino acid
  MOD_RES (67)..(67): Any amino acid
  MOD_RES (71)..(71): Any amino acid
  MOD_RES (78)..(78): Any amino acid
  MOD_RES (96)..(96): Any amino acid
QVQLQESGPGLVKPSETLSLTCTVSGDSITSGYWNWIRQPPGKGLEXXGFISYSGNTYYNPSLRSRXTISXDTSKNQXSLKLSSVTAADTAVYYCXRRHLISGYGWFAYWGQGTLVTVSS

SEQ ID NO: 92 - protein, 109 aa, Artificial Sequence
  Description of Artificial Sequence Synthetic polypeptide
  MOD_RES (1)..(1): Any amino acid
XIVMTQSPDSLAVSLGERATINCKASQDVISAVAWYQQKPGQPPKLLIYWASTRHTGVPDRFSGSGSGTDFTLTISSLQAEDVAVYYCQQHSYTPPWTFGQGTKVEIKR

SEQ ID NO: 93 - protein, 119 aa, Artificial Sequence
  Description of Artificial Sequence Synthetic polypeptide
  MOD_RES (48)..(48): Any amino acid
  MOD_RES (68)..(68): Any amino acid
  MOD_RES (70)..(70): Any amino acid
  MOD_RES (72)..(72): Any amino acid
  MOD_RES (74)..(74): Any amino acid
QVQLVQSGAEVKKPGASVKVSCKASGYTFTSYTMHWVRQAPGQGLEWXGYINPRSVYTNYNQKFKDRXTXTXDXSTSTAYMELRSLRSDDTAVYYCARLGGYYDTMDYWGQGTLVTVSS

SEQ ID NO: 94 - protein, 112 aa, Artificial Sequence
  Description of Artificial Sequence Synthetic polypeptide
DIQMTQSPSSLSASVGDRVTITCRASESVDNYGISFMNWYQQKPGKAPKLLIYAASNQGSGVPSRFSGSGSGTDFTLTISSLQPEDFATYYCQQSKEVPFTFGQGTKVEIKR

SEQ ID NO: 95 - protein, 118 aa, Artificial Sequence
  Description of Artificial Sequence Synthetic polypeptide
  MOD_RES (49)..(49): Any amino acid
  MOD_RES (68)..(68): Any amino acid
  MOD_RES (72)..(72): Any amino acid
QVQLQESGPGLVKPSETLSLTCTVSGYSITSDYAWNWIRQPPGKGLEWXGYISYSGSTRYNPSLKSRXTISXDTSKNQFSLKLSSVTAADTAVYYCARQLGLRFFDYWGQGTLVTVSS

SEQ ID NO: 96 - protein, 107 aa, Artificial Sequence
  Description of Artificial Sequence Synthetic polypeptide
  MOD_RES (45)..(46): Any amino acid
EIVLTQSPGTLSLSPGERATLSCSANSTVNYMYWYQQKPGQAPRXXIYLTSNLASGIPDRFSGSGSGTDFTLTISRLEPEDFAVYYCQQWNSNPPTFGQGTKVEIKR

SEQ ID NO: 97 - protein, 119 aa, Artificial Sequence
  Description of Artificial Sequence Synthetic polypeptide
QVTLRESGPALVKPTQTLTLTCTFSGFSLSTSGMGVGWIRQPPGKALEWLAHIWWDDDKYYNPSLKSRLTISKDTSKNQVVLTMTNMDPVDTATYYCARTRRYFPFAYWGQGTLVTVSS

SEQ ID NO: 98 - protein, 107 aa, Artificial Sequence
  Description of Artificial Sequence Synthetic polypeptide
EIVMTQSPATLSVSPGERATLSCKASQNVGTNVAWYQQKPGQAPRLLIYSASYRYSGIPARFSGSGSGTEFTLTISSLQSEDFAVYYCQQYNTDPLTFGGGTKVEIK

SEQ ID NO: 99 - protein, 119 aa, Artificial Sequence
  Description of Artificial Sequence Synthetic polypeptide
QVTLRESGPALVKPTQTLTLTCTFSGFSLSTSGMGVGWIRQPPGKALEWLAHIWWDDDKYYQPSLKSRLTISKDTSKNQVVLTMTNMDPVDTATYYCARTRRYFPFAYWGQGTLVTVSS

SEQ ID NO: 100 - protein, 121 aa, Artificial Sequence
  Description of Artificial Sequence Synthetic polypeptide
QVQLVESGGGLVQPGGSLRLSCAASGFSLSSYGVDWVRQAPGKGLEWVGVIWGGGGTYYASSVMARFTISRDNSKNTLYLQMNSLRAEDTAVYYCAKHAYGHDGGFAMDYWGQGTLVTVSS

SEQ ID NO: 101 - protein, 107 aa, Artificial Sequence
  Description of Artificial Sequence Synthetic polypeptide
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIK

SEQ ID NO: 102 - protein, 121 aa, Artificial Sequence
  Description of Artificial Sequence Synthetic polypeptide
QVQLVESGGGLVQPGGSLRLSCAASGFSLSSYGVDWVRQAPGKGLEWLGVIWGGGGTYYTASLMGRFTISRDNSKNTLYLQMNSLRAEDTAVYYCAKHAYGHDGGFAMDYWGQGTLVTVSS

SEQ ID NO: 103 - protein, 107 aa, Artificial Sequence
  Description of Artificial Sequence Synthetic polypeptide
EIVMTQSPATLSVSPGERATLSCRASQSVSSNLAWYQQKPGQAPRLLIYGASNRATGIPDRFSGSGSGTDFTLTISRLEPEDFAVYYCGQSYSYPFTFGQGTKLEIK

SEQ ID NO: 104 - protein, 121 aa, Artificial Sequence
  Description of Artificial Sequence Synthetic polypeptide
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCAKHAYGHDGGFAMDYWGQGTLVTVSS

SEQ ID NO: 105 - protein, 121 aa, Artificial Sequence
  Description of Artificial Sequence Synthetic polypeptide
EVQLVESGGGLVQSGGSLRLSCAASGFSLSSYGVDWVRQAPGKGLEWLGVIWGGGGTYYTSSLMGRFTISRDNSKNTLYLQMNSLRAEDTAVYYCAKHAYGHDGGFAMDYWGQGTLVTVSS

SEQ ID NO: 106 - protein, 121 aa, Artificial Sequence
  Description of Artificial Sequence Synthetic polypeptide
EVQLVESGGGLVQSGGSLRLSCAASGFSLSSYGVDWVRQAPGKGLEWLGVIWGGGGTYYTSSLMARFTISRDNSKNTLYLQMNSLRAEDTAVYYCAKHAYGHDGGFAMDYWGQGTLVTVSS

SEQ ID NO: 107 - protein, 121 aa, Artificial Sequence
  Description of Artificial Sequence Synthetic polypeptide
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSS

SEQ ID NO: 108 - protein, 121 aa, Artificial Sequence
  Description of Artificial Sequence Synthetic polypeptide
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARNAYGHDGGFAMDYWGQGTLVTVSS

(19) United States
(12) Patent Application Publication
(10) Pub. No.: US 2022/0177595 A1
(43) Pub. Date: Jun. 9, 2022

(54) MULTIMERIC GITR BINDING MOLECULES AND USES THEREOF

(71) Applicant: IGM Biosciences, Inc., Mountain View, CA (US)

(72) Inventors: Beatrice Tien-Yi WANG, Sunnyvale, CA (US); Bruce Alan KEYT, Hillsborough, CA (US); Ramesh BALIGA, Redwood City, CA (US)

(21) Appl. No.: 17/680,026

(22) Filed: Feb. 24, 2022

Related U.S. Application Data
(63) Continuation of application No. 16/317,816, filed on Jan. 14, 2019, now abandoned.
(60) Provisional application No. 62/364,762, filed on Jul. 20, 2016.

(51) Int. Cl. C07K 16/28; A61P 35/00; C07K 16/46

(52) U.S. Cl. CPC C07K 16/2878; C07K 16/468; A61P 35/00

(57) ABSTRACT

This disclosure provides dimeric, pentameric, and hexameric GITR agonist binding molecules and methods of using such binding molecules to induce anti-tumor immunity.

This application is a continuation of U.S. patent application Ser. No. 16/317,816, filed Jan. 14, 2019, which is a US National Stage Entry of PCT Application No. PCT/US2017/043166, filed Jul. 20, 2017, which claims the benefit of U.S. Provisional Patent Application Ser. No. 62/364,762, filed Jul. 20, 2016, which are all hereby incorporated by referenced in their entireties.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Feb. 23, 2022, is named 007US2-Sequence-Listing and is 144,318 bytes in size.

### BACKGROUND

Tumor Necrosis Factor superfamily receptor (TNFSFR) proteins are important targets for immuno oncology therapeutic agents. For example, agonist monoclonal antibodies directed against TNFSFR targets such as CD40, GITR, CD137, and OX40, among many others, are currently in clinical trials for myriad cancer indications.

In many instances, activation of the TNFSFR targets requires that at least three non-interacting receptor monomers on the surface of a cell expressing the receptor be cross-linked to form a stabilized receptor trimer, resulting in signal transduction across the cell membrane. Clustering of TNFSFR protein trimers into “rafts” of trimers leads to more effective activation of the signaling cascade. (See, Valley et al., J. Biol. Chem., 287(25):21265-21278, 2012). Typically, clustering of TNFSFR on the surface of a cell can be accomplished via engagement by multimeric, e.g., trimeric ligands. Recent work has demonstrated that a multimeric agonistic IgM antibody directed against the TNFSFR DR5 can effectively bind multiple DR5 receptor monomers on the surface of a cell in the absence of secondary cross linking, and with increased cytotoxicity over an IgG molecule with identical binding domains. See PCT Application No. PCT/US16/14153, filed Jan. 20, 2016, which is incorporated herein by reference in its entirety.

Glucocorticoid-Induced TNF Receptor (“GITR,” also known as AITR or TNFRSF18) is a TNFSFR expressed on activated T cells, NK cells, and NKT cells. GITR has low basal expression on naïve murine effector CD4+ and CD8 T+ cells, and very low expression on human effector T cells, e.g., cytotoxic T lymphocytes (CTLs). Murine and human CD4+CD25+ FoxP3+ regulatory T cells (Tregs) constitutively express GITR (Schaer, D A, et al., Curr Opin. Immunol. 24:217-224 (2012)). Upon activation, both effector T cells and Tregs upregulate GITR expression (Id.). Interaction with its trimeric ligand (GITRL, TNFSF18, AITRL) expressed on activated antigen-presenting cells (APCs), e.g., macrophages and dendritic cells (DC), provides enhanced costimulatory proliferation and effector functions in CD4+ and CD8 effector T cells (Tone M, et al., Proc Natl Acad Sci USA. 100:15059-15064 (2003); Ronchetti, S., et al., Eur J. Immunol. 34:613-622 (2004)). GITR signaling can also block the immunosuppressive abilities of Tregs, thereby enhancing cytotoxic T lymphocyte (CTL) function (Shimizu, J., et al., Nature Immunol 3:135-142 (2002)). GITR agonist mAbs can enhance the effector functions and proliferation of CTLs and can impair intratumoral CD25+ CD4+ FoxP3+ Treg stability (Schaer D A, et al. Cancer Immunol Res. 1:320-31 (2013)). Agonist monoclonal antibodies directed against GITR have shown therapeutic activity in preclinical models (See, e.g., Cohen, A D, et al., PLoS One 5(5):e10436. doi: 10.1371/journal.pone.0010436(2010)). Moreover, several GITR IgG agonist mAbs are being investigated in human clinical trials, including, but not limited to TRX518 (humanized agly IgG1) (Schaer, D A, et al., Curr Opin. Immunol. 24:217-224 (2012)); MK-4166 (ClinicalTrials.gov #NCT02132754); and INCAGN1876 (ClinicalTrials.gov #NCT02697591). Typical bivalent IgG agonist antibodies, however, require cross-linking to sufficiently engage TNFSFRs on the surface of a cell to trigger signal transduction.

There remains a need to develop more potent and therefore more effective GITR agonist antibodies for use in cancer immunotherapy.

### SUMMARY

This disclosure provides a multimeric, e.g., dimeric, pentameric, or hexameric binding molecule including two, five, or six bivalent binding units or variants or fragments thereof, where each binding unit includes two IgA or IgM heavy chain constant regions or fragments thereof, each associated with an antigen-binding domain, where at least three of the antigen-binding domains of the binding molecule specifically and agonistically bind to GITR expressed on the surface of activated T cells, e.g., CTLs, or on resting or activated Tregs, where the binding molecule can bind to multiple, e.g., three or more GITR monomers expressed on Tregs or activated CTLs in the absence of a secondary cross-linking moiety, thereby eliciting an anti-tumor immune response.

This disclosure provides a multimeric binding molecule that includes two, five, or six bivalent binding units or variants or fragments thereof, where each binding unit includes two IgA or IgM heavy chain constant regions or fragments thereof, each associated with an antigen-binding domain, where at least three of the antigen-binding domains of the binding molecule can specifically and agonistically bind to a GITR monomer on a cell expressing GITR, and where the binding molecule can induce GITR-mediated signal transduction in the cell in the absence of a secondary cross-linking moiety. In certain aspects, the multimeric binding molecule can bind to and engage three or more GITR monomers expressed on the surface of the cell in the absence of a secondary cross-linking moiety.

In certain aspects the cell expressing GITR is a T cell, e.g., a cytotoxic T lymphocyte (CTL), and GITR mediated signal transduction in the cell can, e.g., increase surface expression of GITR, increase CTL proliferation, increase production of proinflammatory cytokines, increase resistance to the inhibitory effects of CD4+ CD25+ FoxP3+ Treg cells, increase or enhance killing of tumor cells, or any combination thereof. In certain aspects the T cell is a CD4+ CD25+ FoxP3+ Treg cell, and GITR-mediated signal transduction in the cell can, e.g., interfere with the cell's ability to suppress anti-tumor immunity in the tumor microenvironment.

In certain aspects, the multimeric binding molecule provided herein can induce GITR-mediated signal transduction in the cell expressing GITR at a higher potency than an equivalent amount of a bivalent IgG antibody or fragment thereof that includes two equivalent GITR antigen-binding domains. In certain aspects the multimeric binding molecule provided herein includes at least three, at least four, at least five, at least six, at least seven, at least eight, at least nine, at least ten, at least eleven, or twelve antigen-binding domains that specifically and agonistically bind to a GITR monomer expressed on the surface of the cell, thereby activating GITR-mediated signal transduction in the cell. In certain aspects the three, four, five, six, seven, eight, nine, ten, eleven, or twelve antigen-binding domains bind to the same extracellular GITR epitope. In certain aspects the three, four, five, six, seven, eight, nine, ten, eleven, or twelve antigen-binding domains each specifically bind one of a group of two or more different extracellular GITR epitopes.

In certain aspects the two, five, or six binding units of the multimeric binding molecule provided herein are human, humanized, or chimeric immunoglobulin binding units.

In certain aspects at the least three antigen-binding domains of the multimeric binding molecule provided herein are GITR agonist binding domains, and at least one, at least two, at least three, at least four, at least five, at least six, at least seven, at least eight, at least nine, at least ten, at least eleven, or twelve of the antigen-binding domains include a heavy chain variable region (VH) and a light chain variable region (VL), where the VH and VL include six immunoglobulin complementarity determining regions HCDR1, HCDR2, HCDR3, LCDR1, LCDR2, and LCDR3 that include the six CDRs of an antibody that includes the VH and VL amino acid sequences comprising or contained within SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 15 and SEQ ID NO: 17; SEQ ID NO: 18 and SEQ ID NO: 19; SEQ ID NO: 20 and SEQ ID NO: 21; SEQ ID NO: 22 and SEQ ID NO: 23; SEQ ID NO: 22 and SEQ ID NO: 24; SEQ ID NO: 25 and SEQ ID NO: 26; SEQ ID NO: 27 and SEQ ID NO: 28; SEQ ID NO: 27 and SEQ ID NO: 29; SEQ ID NO: 30 and SEQ ID NO: 31; SEQ ID NO: 32 and SEQ ID NO: 33; SEQ ID NO: 32 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 57 and SEQ ID NO: 58; SEQ ID NO: 59 and SEQ ID NO: 60; SEQ ID NO: 61 and SEQ ID NO: 62; SEQ ID NO: 63 and SEQ ID NO: 64; SEQ ID NO: 65 and SEQ ID NO: 66; SEQ ID NO: 67 and SEQ ID NO: 68; SEQ ID NO: 69 and SEQ ID NO: 68; SEQ ID NO: 70 and SEQ ID NO: 71; SEQ ID NO: 72 and SEQ ID NO: 71; SEQ ID NO: 73 and SEQ ID NO: 74; SEQ ID NO: 75 and SEQ ID NO: 76; SEQ ID NO: 77 and SEQ ID NO: 78; SEQ ID NO: 79 and SEQ ID NO: 80; SEQ ID NO: 81 and SEQ ID NO: 82; SEQ ID NO: 83 and SEQ ID NO: 84; SEQ ID NO: 85 and SEQ ID NO: 86; SEQ ID NO: 87 and SEQ ID NO: 88; SEQ ID NO: 89 and SEQ ID NO: 90; SEQ ID NO: 91 and SEQ ID NO: 92; SEQ ID NO: 93 and SEQ ID NO: 94; SEQ ID NO: 95 and SEQ ID NO: 96; SEQ ID NO: 97 and SEQ ID NO: 98; SEQ ID NO: 99 and SEQ ID NO: 98; SEQ ID NO: 100 and SEQ ID NO: 101; SEQ ID NO: 102 and SEQ ID NO: 103; SEQ ID NO: 104 and SEQ ID NO: 101; SEQ ID NO: 105 and SEQ ID NO: 101; SEQ ID NO: 106 and SEQ ID NO: 101; SEQ ID NO: 107 and SEQ ID NO: 101; SEQ ID NO: 108 and SEQ ID NO: 101; or SEQ ID NO: 109 and SEQ ID NO: 110, respectively or the CDRs of an antibody that includes the VH and VL amino acid sequences comprising or contained within SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 15 and SEQ ID NO: 17; SEQ ID NO: 18 and SEQ ID NO: 19; SEQ ID NO: 20 and SEQ ID NO: 21; SEQ ID NO: 22 and SEQ ID NO: 23; SEQ ID NO: 22 and SEQ ID NO: 24; SEQ ID NO: 25 and SEQ ID NO: 26; SEQ ID NO: 27 and SEQ ID NO: 28; SEQ ID NO: 27 and SEQ ID NO: 29; SEQ ID NO: 30 and SEQ ID NO: 31; SEQ ID NO: 32 and SEQ ID NO: 33; SEQ ID NO: 32 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 57 and SEQ ID NO: 58; SEQ ID NO: 59 and SEQ ID NO: 60; SEQ ID NO: 61 and SEQ ID NO: 62; SEQ ID NO: 63 and SEQ ID NO: 64; SEQ ID NO: 65 and SEQ ID NO: 66; SEQ ID NO: 67 and SEQ ID NO: 68; SEQ ID NO: 69 and SEQ ID NO: 68; SEQ ID NO: 70 and SEQ ID NO: 71; SEQ ID NO: 72 and SEQ ID NO: 71; SEQ ID NO: 73 and SEQ ID NO: 74; SEQ ID NO: 75 and SEQ ID NO: 76; SEQ ID NO: 77 and SEQ ID NO: 78; SEQ ID NO: 79 and SEQ ID NO: 80; SEQ ID NO: 81 and SEQ ID NO: 82; SEQ ID NO: 83 and SEQ ID NO: 84; SEQ ID NO: 85 and SEQ ID NO: 86; SEQ ID NO: 87 and SEQ ID NO: 88; SEQ ID NO: 89 and SEQ ID NO: 90; SEQ ID NO: 91 and SEQ ID NO: 92; SEQ ID NO: 93 and SEQ ID NO: 94; SEQ ID NO: 95 and SEQ ID NO: 96; SEQ ID NO: 97 and SEQ ID NO: 98; SEQ ID NO: 99 and SEQ ID NO: 98; SEQ ID NO: 100 and SEQ ID NO: 101; SEQ ID NO: 102 and SEQ ID NO: 103; SEQ ID NO: 104 and SEQ ID NO: 101; SEQ ID NO: 105 and SEQ ID NO: 101; SEQ ID NO: 106 and SEQ ID NO: 101; SEQ ID NO: 107 and SEQ ID NO: 101; SEQ ID NO: 108 and SEQ ID NO: 101; or SEQ ID NO: 109 and SEQ ID NO: 110, respectively, except for one or two amino acid substitutions in one or more of the six CDRs.

In certain aspects, at least one, at least two, at least three, at least four, at least five, at least six, at least seven, at least eight, at least nine, at least ten, at least eleven, or twelve antigen-binding domains of the multimeric binding molecule provided herein include an antibody VH and a VL, where the VH and VL have amino acid sequences at least 80%, at least 85%, at least 90%, at least 95% or 100% identical to the mature VH and VL amino acid sequences comprising or contained within SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 15 and SEQ ID NO: 17; SEQ ID NO: 18 and SEQ ID NO: 19; SEQ ID NO: 20 and SEQ ID NO: 21; SEQ ID NO: 22 and SEQ ID NO: 23; SEQ ID NO: 22 and SEQ ID NO: 24; SEQ ID NO: 25 and SEQ ID NO: 26; SEQ ID NO: 27 and SEQ ID NO: 28; SEQ ID NO: 27 and SEQ ID NO: 29; SEQ ID NO: 30 and SEQ ID NO: 31; SEQ ID NO: 32 and SEQ ID NO: 33; SEQ ID NO: 32 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 57 and SEQ ID NO: 58; SEQ ID NO: 59 and SEQ ID NO: 60; SEQ ID NO: 61 and SEQ ID NO: 62; SEQ ID NO: 63 and SEQ ID NO: 64; SEQ ID NO: 65 and SEQ ID NO: 66; SEQ ID NO: 67 and SEQ ID NO: 68; SEQ ID NO: 69 and SEQ ID NO: 68; SEQ ID NO: 70 and SEQ ID NO: 71; SEQ ID NO: 72 and SEQ ID NO: 71; SEQ ID NO: 73 and SEQ ID NO: 74; SEQ ID NO: 75 and SEQ ID NO: 76; SEQ ID NO: 77 and SEQ ID NO: 78; SEQ ID NO: 79 and SEQ ID NO: 80; SEQ ID NO: 81 and SEQ ID NO: 82; SEQ ID NO: 83 and SEQ ID NO: 84; SEQ ID NO: 85 and SEQ ID NO: 86; SEQ ID NO: 87 and SEQ ID NO: 88; SEQ ID NO: 89 and SEQ ID NO: 90; SEQ ID NO: 91 and SEQ ID NO: 92; SEQ ID NO: 93 and SEQ ID NO: 94; SEQ ID NO: 95 and SEQ ID NO: 96; SEQ ID NO: 97 and SEQ ID NO: 98; SEQ ID NO: 99 and SEQ ID NO: 98; SEQ ID NO: 100 and SEQ ID NO: 101; SEQ ID NO: 102 and SEQ ID NO: 103; SEQ ID NO: 104 and SEQ ID NO: 101; SEQ ID NO: 105 and SEQ ID NO: 101; SEQ ID NO: 106 and SEQ ID NO: 101; SEQ ID NO: 107 and SEQ ID NO: 101; SEQ ID NO: 108 and SEQ ID NO: 101; or SEQ ID NO: 109 and SEQ ID NO: 110, respectively.

In certain aspects the multimeric binding molecule provided herein is a dimeric binding molecule that includes two bivalent IgA binding units or fragments thereof and a J chain or fragment or variant thereof, where each binding unit includes two IgA heavy chain constant regions or fragments thereof each associated with an antigen-binding domain. In certain aspects this binding molecule can further include a secretory component, or fragment or variant thereof. In certain aspects the IgA heavy chain constant regions or fragments thereof each include a Cα2 domain or a Cα3-tp domain, and can further include a Cα1 domain. In certain aspects the IgA heavy chain constant region is a human IgA constant region. In certain aspects each binding unit of this binding molecule can include two IgA heavy chains each having a VH situated amino terminal to the IgA constant region or fragment thereof, and two immunoglobulin light chains each having a VL situated amino terminal to an immunoglobulin light chain constant region.

In certain aspects the multimeric binding molecule provided herein is a pentameric or a hexameric binding molecule that includes five or six bivalent IgM binding units, respectively, where each binding unit includes two IgM heavy chain constant regions or fragments thereof each associated with an antigen-binding domain. Where the multimeric binding molecule is a pentameric IgM molecule, it can further include a J chain or fragment or variant thereof.

In certain aspects the IgM heavy chain constant regions or fragments thereof each include a Cμ3 domain or fragment or variant thereof and a Cμ4-tp domain or fragment or variant thereof, and can further include a Cμ2 domain, a Cμ1 domain, or any combination thereof. In certain aspects, this multimeric binding molecule is pentameric, and further includes a J chain, or fragment thereof, or variant thereof. In certain aspects, the IgM heavy chain constant region of this multimeric binding molecule is a human IgM constant region. In certain aspects each binding unit of this multimeric binding molecule includes two IgM heavy chains each having a VH situated amino terminal to the IgM constant region or fragment thereof, and two immunoglobulin light chains each having a VL situated amino terminal to an immunoglobulin light chain constant region.

In certain aspects, each binding unit of the multimeric binding molecule provided herein includes two heavy chains and two light chains, where the heavy chains and light chains include VH and VL amino acid sequences at least 80%, at least 85%, at least 90%, at least 95% or 100% identical to the VH and VL amino acid sequences comprising or contained within SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 15 and SEQ ID NO: 17; SEQ ID NO: 18 and SEQ ID NO: 19; SEQ ID NO: 20 and SEQ ID NO: 21; SEQ ID NO: 22 and SEQ ID NO: 23; SEQ ID NO: 22 and SEQ ID NO: 24; SEQ ID NO: 25 and SEQ ID NO: 26; SEQ ID NO: 27 and SEQ ID NO: 28; SEQ ID NO: 27 and SEQ ID NO: 29; SEQ ID NO: 30 and SEQ ID NO: 31; SEQ ID NO: 32 and SEQ ID NO: 33; SEQ ID NO: 32 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 57 and SEQ ID NO: 58; SEQ ID NO: 59 and SEQ ID NO: 60; SEQ ID NO: 61 and SEQ ID NO: 62; SEQ ID NO: 63 and SEQ ID NO: 64; SEQ ID NO: 65 and SEQ ID NO: 66; SEQ ID NO: 67 and SEQ ID NO: 68; SEQ ID NO: 69 and SEQ ID NO: 68; SEQ ID NO: 70 and SEQ ID NO: 71; SEQ ID NO: 72 and SEQ ID NO: 71; SEQ ID NO: 73 and SEQ ID NO: 74; SEQ ID NO: 75 and SEQ ID NO: 76; SEQ ID NO: 77 and SEQ ID NO: 78; SEQ ID NO: 79 and SEQ ID NO: 80; SEQ ID NO: 81 and SEQ ID NO: 82; SEQ ID NO: 83 and SEQ ID NO: 84; SEQ ID NO: 85 and SEQ ID NO: 86; SEQ ID NO: 87 and SEQ ID NO: 88; SEQ ID NO: 89 and SEQ ID NO: 90; SEQ ID NO: 91 and SEQ ID NO: 92; SEQ ID NO: 93 and SEQ ID NO: 94; SEQ ID NO: 95 and SEQ ID NO: 96; SEQ ID NO: 97 and SEQ ID NO: 98; SEQ ID NO: 99 and SEQ ID NO: 98; SEQ ID NO: 100 and SEQ ID NO: 101; SEQ ID NO: 102 and SEQ ID NO: 103; SEQ ID NO: 104 and SEQ ID NO: 101; SEQ ID NO: 105 and SEQ ID NO: 101; SEQ ID NO: 106 and SEQ ID NO: 101; SEQ ID NO: 107 and SEQ ID NO: 101; SEQ ID NO: 108 and SEQ ID NO: 101; or SEQ ID NO: 109 and SEQ ID NO: 110, respectively.

The disclosure further provides a composition that includes the multimeric binding molecule provided herein.

The disclosure further provides a polynucleotide that includes a nucleic acid sequence encoding a polypeptide subunit of the multimeric binding molecule provided herein.

In certain aspects the polypeptide subunit includes an IgM heavy chain constant region and at least an antibody VH portion of the antigen-binding domain of the multimeric binding molecule. In certain aspects the polypeptide subunit includes a human IgM constant region or fragment thereof fused to the C-terminal end of a VH that includes the HCDR1, HCDR2, and HCDR3 regions contained in the VH amino acid sequence comprising or contained within SEQ ID NO: 9, SEQ ID NO: 11, SEQ ID NO: 13, SEQ ID NO: 15, SEQ ID NO: 18, SEQ ID NO: 20, SEQ ID NO: 22, SEQ ID NO: 25, SEQ ID NO: 27, SEQ ID NO: 30, SEQ ID NO: 32, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 70, SEQ ID NO: 72, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 100, SEQ ID NO: 102, SEQ ID NO: 104, SEQ ID NO: 105, SEQ ID NO: 106, SEQ ID NO: 107, SEQ ID NO: 108, or SEQ ID NO: 109; or the HCDR1, HCDR2, and HCDR3 regions contained in the VH amino acid sequence comprising or contained within SEQ ID NO: 9, SEQ ID NO: 11, SEQ ID NO: 13, SEQ ID NO: 15, SEQ ID NO: 18, SEQ ID NO: 20, SEQ ID NO: 22, SEQ ID NO: 25, SEQ ID NO: 27, SEQ ID NO: 30, SEQ ID NO: 32, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 70, SEQ ID NO: 72, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 100, SEQ ID NO: 102, SEQ ID NO: 104, SEQ ID NO: 105, SEQ ID NO: 106, SEQ ID NO: 107, SEQ ID NO: 108, or SEQ ID NO: 109 except for one or two single amino acid substitutions in one or more of the HCDRs; and/or an amino acid sequence at least 80%, at least 85%, at least 90%, at least 95% or 100% identical to the mature VH amino acid sequence comprising or contained within SEQ ID NO: 9, SEQ ID NO: 11, SEQ ID NO: 13, SEQ ID NO: 15, SEQ ID NO: 18, SEQ ID NO: 20, SEQ ID NO: 22, SEQ ID NO: 25, SEQ ID NO: 27, SEQ ID NO: 30, SEQ ID NO: 32, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 70, SEQ ID NO: 72, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 100, SEQ ID NO: 102, SEQ ID NO: 104, SEQ ID NO: 105, SEQ ID NO: 106, SEQ ID NO: 107, SEQ ID NO: 108, or SEQ ID NO: 109.

In certain aspects the polypeptide subunit includes a light chain constant region and an antibody VL portion of the antigen-binding domain of the multimeric binding molecule. In certain aspects the polypeptide subunit include a human kappa or lambda light chain constant region or fragment thereof fused to the C-terminal end of a VL that includes LCDR1, LCDR2, and LCDR3 regions contained in the VL amino acid sequence comprising or contained within SEQ ID NO: 10, SEQ ID NO: 12, SEQ ID NO: 14, SEQ ID NO: 16, SEQ ID NO: 17, SEQ ID NO: 19, SEQ ID NO: 21, SEQ ID NO: 23, SEQ ID NO: 24, SEQ ID NO: 26, SEQ ID NO: 28, SEQ ID NO: 29, SEQ ID NO: 31, SEQ ID NO: 33, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 71, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 101, SEQ ID NO: 103, or SEQ ID NO: 110; LCDR1, LCDR2, and LCDR3 regions contained in the VL amino acid sequence comprising or contained within SEQ ID NO: 10, SEQ ID NO: 12, SEQ ID NO: 14, SEQ ID NO: 16, SEQ ID NO: 17, SEQ ID NO: 19, SEQ ID NO: 21, SEQ ID NO: 23, SEQ ID NO: 24, SEQ ID NO: 26, SEQ ID NO: 28, SEQ ID NO: 29, SEQ ID NO: 31, SEQ ID NO: 33, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 71, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 101, SEQ ID NO: 103, or SEQ ID NO: 110 except for one or two single amino acid substitutions in one or more of the LCDRs; and/or an amino acid sequence at least 80%, at least 85%, at least 90%, at least 95% or 100% identical to the mature VL amino acid sequence comprising or contained within SEQ ID NO: 10, SEQ ID NO: 12, SEQ ID NO: 14, SEQ ID NO: 16, SEQ ID NO: 17, SEQ ID NO: 19, SEQ ID NO: 21, SEQ ID NO: 23, SEQ ID NO: 24, SEQ ID NO: 26, SEQ ID NO: 28, SEQ ID NO: 29, SEQ ID NO: 31, SEQ ID NO: 33, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 71, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 101, SEQ ID NO: 103, or SEQ ID NO: 110.

The disclosure further provides a composition that includes a polynucleotide encoding a VH and a polynucleotide encoding a VL. In certain aspects the polynucleotides are on separate vectors. In certain aspects the polynucleotides are on a single vector. In certain aspects the composition further includes a polynucleotide that includes a nucleic acid sequence encoding a J chain, or fragment thereof, or variant thereof, that can be on the same or on a separate vector relative to the VH and/or the VL. This vector or these vectors are also provided.

The disclosure further provides a host cell that includes one or more of the provided polynucleotides, the provided composition, and/or the provided vector or vectors. In certain aspects the provided host cell can express the multimeric binding molecule provided herein. The disclosure further provides a method of producing the multimeric binding molecule provided herein, where the method includes culturing the provided host cell and recovering the binding molecule.

The disclosure further provides a method of inducing GITR-mediated activation in a GITR-expressing cell, where the method includes contacting the GITR-expressing cell with the multimeric binding molecule provided herein.

The disclosure further provides a method of inducing GITR translocation and clustering in GITR-expressing T cells, where the method includes contacting GITR-expressing T cells with the multimeric binding molecule provided herein.

The disclosure further provides a method of treating cancer where the method includes administering to a subject in need of treatment an effective amount of the multimeric binding molecule provided herein, where the multimeric binding molecule can activate GITR-expressing CTL thereby triggering a tumoricidal CTL response. In certain aspects the subject is human. In another aspect the disclosure provides use of the multimeric binding molecule provided herein in the preparation of a medicament for treating cancer, where the multimeric binding molecule can activate GITR-expressing CTL thereby triggering a tumoricidal CTL response. In another aspect the disclosure provides the multimeric binding molecule provided herein for use in treating cancer.

### BRIEF DESCRIPTION OF THE DRAWINGS

FIG. 1A-1C: Generation of anti-GITR IgMs. FIG. 1A. Non-reduced gel shows high molecular weight IgMs. FIG. 1B. Reduced gel shows IgM heavy and light chains.

FIG. 1C. Anti-J chain western blot confirms presence of J chain in IgM pentamer. M, Molecular weight standard; 1, Anti-GITR IgM #1; 2, Anti-GITR IgM #2.

FIG. 2A-2D: The specificity of the IgG and IgM versions of Anti-GITR #1 and Anti-GITR #2 for human GITR was measured in an ELISA assay at two different antigen densities. FIG. 2A-B: Anti-GITR IgG or IgM #1 binding at 10 ng/ml antigen density (FIG. 2A), or 1 ng/ml antigen density (FIG. 2B); FIG. 2C-D: Anti-GITR IgG or IgM #2 binding at 10 ng/ml antigen density (FIG. 2C), or 1 ng/ml antigen density (FIG. 2D). IgG, open circles; IgM, filled squares.

FIG. 3A-3D: Anti-GITR IgG and IgM antibodies bind GITR on activated T cells. T cells were activated with 5 μg/mL anti-CD3 and 2 μg/mL anti-CD28 for 4 days, and GITR binding on CD4 T cells was measured for 5 μg/mL anti-GITR IgG and IgM #1 (FIG. 3A) and 5 μg/mL anti-GITR IgG and IgM #2 (FIG. 3B). Filled histograms, Isotype controls; Open histograms, Anti-GITR antibodies. Anti-GITR IgG and IgM #1 (FIG. 3C) and Anti-GITR IgG and IgM #2 (FIG. 3D) binding dose response on activated T cells. Closed sequares: IgM; open circles: IgG; x: IgG isotype control; star: IgM isotype control.

FIG. 4A-4B: Induction of GITR signaling. Anti-GITR IgM and IgG induced signaling were measured in NFκB-luc2/GITR Jurkat reporter cells. FIG. 4A: anti-GITR #1; FIG. 4B: anti-GITR #2. Closed squares: IgM; open circles, IgG; closed circles: IgG+ crosslinker; x: IgG isotype control; star: IgM isotype control.

FIG. 5A-5D: Anti-GITR IgM enhances T cell activation. FIGS. 5A and 5B: cytokine production by Human CD4 T cells activated with a suboptimal (0.6 μg/mL) (FIG. 5A) or a high (3 μg/mL) (FIG. 5B) dose of plate bound anti-CD3 (clone OKT3) along with anti-CD28 and anti-GITR antibodies. FIG. 5C-5D: T cells were activated as above, but 10 μg/mL of anti-human IgG Fc cross-linker was additionally plate-coated prior to seeding cells in the anti-GITR IgG #1 plus cross-linker samples. IFN□ in the supernatant was measured by ELISA. White bars, No Anti-GITR antibody; Gray bars, Anti-GITR IgG #1; Patterned gray bars, Anti-GITR IgG #1 plus cross-linker; Black bars, Anti-GITR IgM #1.

### DETAILED DESCRIPTION

### Definitions

It is to be noted that the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology, but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain aspects, variants can be non-naturally occurring. Non-naturally occurring variants can be produced using art-known mutagenesis techniques. Variant polypeptides can comprise conservative or non-conservative amino acid substitutions, deletions or additions. Derivatives are polypeptides that have been altered so as to exhibit additional features not found on the original polypeptide. Examples include fusion proteins. Variant polypeptides can also be referred to herein as “polypeptide analogs.” As used herein a “derivative” of a polypeptide can also refer to a subject polypeptide having one or more amino acids chemically derivatized by reaction of a functional side group. Also included as “derivatives” are those peptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the present disclosure do not abrogate the binding of the polypeptide or antibody containing the amino acid sequence, to the antigen to which the binding molecule binds. Methods of identifying nucleotide and amino acid conservative substitutions which do not eliminate antigen-binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94: 412-417 (1997)).

The term “polynucleotide” is intended to encompass a singular nucleic acid as well as plural nucleic acids, and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, the term “a non-naturally occurring polynucleotide” or any grammatical variants thereof, is a conditional definition that explicitly excludes, but only excludes, those forms of the nucleic acid or polynucleotide that are, or might be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit B-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

As used herein, the terms “TNF superfamily receptor proteins,” “TNFSFR,” “TNF receptor family,” “TNF receptors” or any combination of such phrases, refer to the family of Tumor Necrosis Factor transmembrane receptor proteins expressed on the surface of various cells and tissues. Family members of this superfamily include those that, upon activation by ligand binding or agonist antibody binding can trigger: activation, an inflammatory response, apoptosis (or inhibit apoptosis), proliferation, and/or morphogenesis in a cell in which the receptor protein is expressed. TNFSFRs include, but are not limited to TNFR1 (DR1), TNFR2, TNFR1/2, CD40 (p50), Fas (CD95, Apo1, DR2), CD30, 4-1BB (CD137, ILA), TRAILR1 (DR4, Apo2), TRAILR2 (DR5), TRAILR3 (DcR1), TRAILR4 (DcR2), OPG (OCIF), TWEAKR (FN14), LIGHTR (HVEM), DcR3, DR3, EDAR, XEDAR, LT-βR, GITR (AITR), TACI, BCMA, CD27, OX40 (CD134), RANK (TRANCER), RELT, and BAFF-R. See, e.g., Wajant, H. Cell Death and Differentiation 22:1727-1741 (2015).

Disclosed herein are certain binding molecules, or antigen-binding fragments, variants, or derivatives thereof that agonistically bind to the TNFSFR GITR, and can thereby elicit, e.g., proliferation and enhanced effector function in activated CTLs expressing GITR, and impairment of immune suppression by CD25+ CD4+ FoxP3+ Tregs, e.g., in the microenvironment surrounding a tumor, thus promoting anti-tumor immunity. Unless specifically referring to full-sized antibodies, the term “binding molecule” encompasses full-sized antibodies as well as antigen-binding subunits, fragments, variants, analogs, or derivatives of such antibodies, e.g., engineered antibody molecules or fragments that bind antigen in a manner similar to antibody molecules, but which use a different scaffold.

The precursor form of isoform 1 of human GITR comprises the amino acid sequence SEQ ID NO: 7 (UniProtKB/Swiss-Prot: 035714.1). Other isoforms share significant homology with SEQ ID NO: 7. The mature protein includes amino acids 26 to 241 of SEQ ID NO: 7, with amino acids 1-25 comprising the signal peptide. The extracellular domain of human GITR includes amino acids 26 to 162 of SEQ ID NO: 7. The transmembrane domain of human GITR includes amino acids 163 to 183 of SEQ ID NO: 7. The cytoplasmic domain of human GITR includes amino acids 184 to 241 of SEQ ID NO: 7. SEQ ID NO: 7:

MAQHGAMGAFRALCGLALLCALSLGQRPTGGPGCG
PGRLLLGTGTDARCCRVHTTRCCRDYPGEECCSEWD
CMCVQPEFHCGDPCCTTCRHHPCPPGQGVQSQGKFS
FGFQCIDCASGTFSGGHEGHCKPWTDCTQFGFLTVFP
GNKTHNAVCVPGSPPAEPLGWLTVVLLAVAACVLLL
TSAQLGLHIWQLRSQCMWPRETQLLLEVPPSTEDARS
CQFPEEERGERSAEEKGRLGDLWV

The precursor form of murine GITR comprises the amino acid sequence SEQ ID NO: 8 (UniProtKB/Swiss-Prot: 035714.1). Other isoforms share significant homology with SEQ ID NO: 8. The mature protein includes amino acids 20 to 228 of SEQ ID NO: 8, with amino acids 1-19 comprising the signal peptide. The extracellular domain of murine GITR includes amino acids 20 to 153 of SEQ ID NO: 8. The transmembrane domain of murine GITR includes amino acids 154 to 174 of SEQ ID NO: 8. The cytoplasmic domain of murine GITR includes amino acids 175 to 228 of SEQ ID NO: 8. SEQ ID NO: 8:

MGAWAMLYGVSMLCVLDLGQPSVVEEPGCGPGKVQ
NGSGNNTRCCSLYAPGKEDCPKERCICVTPEYHCGDP
QCKICKHYPCQPGQRVESQGDIVFGFRCVACAMGTFS
AGRDGHCRLWTNCSQFGFLTMFPGNKTHNAVCIPEP
LPTEQYGHLTVIFLVMAACIFFLTTVQLGLHIWQLRR
QHMCPRETQPFAEVQLSAEDACSFQFPEEERGEQTEE
KCHLGGRWP

As used herein, the term “binding molecule” refers in its broadest sense to a molecule that specifically binds to a receptor, e.g., an epitope or an antigenic determinant. As described further herein, a binding molecule can comprise one of more “antigen binding domains” described herein. A non-limiting example of a binding molecule is an antibody or fragment thereof that retains antigen-specific binding.

As used herein, the terms “binding domain” or “antigen binding domain” refer to a region of a binding molecule that is necessary and sufficient to specifically bind to an epitope. For example, an “Fv,” e.g., a variable heavy chain and variable light chain of an antibody, either as two separate polypeptide subunits or as a single chain, is considered to be a “binding domain.” Other binding domains include, without limitation, the variable heavy chain (VHH) of an antibody derived from a camelid species, or six immunoglobulin complementarity determining regions (CDRs) expressed in a fibronectin scaffold. A “binding molecule” as described herein can include one, two, three, four, five, six, seven, eight, nine, ten, eleven, twelve or more “antigen binding domains.”

The terms “antibody” and “immunoglobulin” can be used interchangeably herein. An antibody (or a fragment, variant, or derivative thereof as disclosed herein) includes at least the variable domain of a heavy chain (for camelid species) or at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody that includes four complete heavy chains and four complete light chains and optionally includes a J chain and/or a secretory component, or an IgM antibody that includes ten or twelve complete heavy chains and ten or twelve complete light chains and optionally includes a J chain.

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2)). It is the nature of this chain that determines the “isotype” of the antibody as IgG, IgM, IgA IgG, or IgE, respectively. The immunoglobulin subclasses (subtypes) e.g., IgG₁, IgG₂, IgG₃, IgG₄, IgA₁, IgA₂, etc. are well characterized and are known to confer functional specialization. Modified versions of each of these immunoglobulins are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

The term “binding unit” is used herein to refer to the portion of a binding molecule, e.g., an antibody or antigen-binding fragment thereof, which corresponds to a standard “H2L2” immunoglobulin structure, i.e., two heavy chains or fragments thereof and two light chains or fragments thereof. In certain aspects, e.g., where the binding molecule is a bivalent IgG antibody or antigen-binding fragment thereof, the terms “binding molecule” and “binding unit” are equivalent. In other aspects, e.g., where the binding molecule is an IgA dimer, an IgM pentamer, or an IgM hexamer, the binding molecule comprises two or more “binding units.” Two in the case of an IgA dimer, or five or six in the case of an IgM pentamer or hexamer, respectively. A binding unit need not include full-length antibody heavy and light chains, but will typically be bivalent, i.e., will include two “binding domains,” as defined above. As used herein, certain binding molecules provided in this disclosure are “dimeric,” and include two bivalent binding units that include IgA constant regions or fragments thereof. Certain binding molecules provided in this disclosure are “pentameric” or “hexameric,” and include five or six bivalent binding units that include IgM constant regions or fragments thereof. A binding molecule comprising two or more, e.g., two, five, or six binding units, is referred to herein as “multimeric.”

The terms “valency,” “bivalent,” “multivalent” and grammatical equivalents, refer to the number of binding domains in given binding molecule or binding unit. As such, the terms “bivalent”, “tetravalent”, and “hexavalent” in reference to a given binding molecule, e.g., an IgM antibody or fragment thereof, denote the presence of two binding domains, four binding domains, and six binding domains, respectively. In a typical IgM-derived binding molecule where each binding unit is bivalent, the binding molecule itself can have 10 or 12 valencies. A bivalent or multivalent binding molecule can be monospecific, i.e., all of the binding domains are the same, or can be bispecific or multispecific, e.g., where two or more binding domains are different, e.g., bind to different epitopes on the same antigen, or bind to entirely different antigens.

The term “epitope” includes any molecular determinant capable of specific binding to an antibody. In certain aspects, an epitope can include chemically active surface groupings of molecules such as amino acids, sugar side chains, phosphoryl, or sulfonyl, and, in certain aspects, can have a three dimensional structural characteristics, and or specific charge characteristics. An epitope is a region of a target that is bound by an antibody.

The term “target” is used in the broadest sense to include substances that can be bound by a binding molecule. A target can be, e.g., a polypeptide, a nucleic acid, a carbohydrate, a lipid, or other molecule. Moreover, a “target” can, for example, be a cell, an organ, or an organism that comprises an epitope bound that can be bound by a binding molecule.

Both the light and heavy chains are divided into regions of structural and functional homology. The terms “constant” and “variable” are used functionally. In this regard, it will be appreciated that the variable domains of both the variable light (VL) and variable heavy (VH) chain portions determine antigen recognition and specificity. Conversely, the constant domains of the light chain (CL) and the heavy chain (e.g., CH1, CH2 or CH3) confer biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 (or CH4 in the case of IgM) and CL domains actually comprise the carboxy-terminus of the heavy and light chain, respectively.

A “full length IgM antibody heavy chain” is a polypeptide that includes, in N-terminal to C-terminal direction, an antibody heavy chain variable domain (VH), an antibody constant heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a tailpiece.

A “full length IgA antibody heavy chain” is a polypeptide that includes, in N-terminal to C-terminal direction, an antibody heavy chain variable domain (VH), an antibody constant heavy chain constant domain 1 (CA1 or Cα1), an antibody heavy chain constant domain 2 (CA2 or Cα2), and an antibody heavy chain constant domain 3 (CA3 or Cα3) that can include a tailpiece.

As indicated above, variable region(s) allows a binding molecule to selectively recognize and specifically bind epitopes on antigens. That is, the VL domain and VH domain, or subset of the complementarity determining regions (CDRs), of a binding molecule, e.g., an antibody, combine to form the antigen binding domain. More specifically, an antigen binding domain can be defined by three CDRs on each of the VH and VL chains. Certain antibodies form larger structures. For example, IgA can form a molecule that includes two H2L2 binding units and a J chain covalently connected via disulfide bonds, which can be further associated with a secretory component, and IgM can form a pentameric or hexameric molecule that includes five or six H2L2 binding units and optionally a J chain covalently connected via disulfide bonds.

The six “complementarity determining regions” or “CDRs” present in an antibody antigen-binding domain are short, non-contiguous sequences of amino acids that are specifically positioned to form the binding domain as the antibody assumes its three dimensional configuration in an aqueous environment. The remainder of the amino acids in the binding domain, referred to as “framework” regions, show less inter-molecular variability. The framework regions largely adopt a n-sheet conformation and the CDRs form loops which connect, and in some cases form part of, the n-sheet structure. Thus, framework regions act to form a scaffold that provides for positioning the CDRs in correct orientation by inter-chain, non-covalent interactions. The binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

Binding molecules, e.g., antibodies or antigen-binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019.

By “specifically binds,” it is generally meant that a binding molecule, e.g., an antibody or fragment, variant, or derivative thereof binds to an epitope via its antigen binding domain, and that the binding entails some complementarity between the antigen binding domain and the epitope. According to this definition, a binding molecule is said to “specifically bind” to an epitope when it binds to that epitope, via its antigen binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k(off)) of less than or equal to 5×10⁻² sec⁻¹, 10⁻² sec⁻¹, 5×10⁻³ sec⁻¹, 10⁻³ sec⁻¹, 5×10⁻⁴ sec⁻¹, 10⁻⁴ sec⁻¹, 5×10⁻⁵ sec⁻¹, or 10⁻⁵ sec⁻¹5×10⁻⁶ sec⁻¹, 10⁻⁶ sec⁻¹, 5×10⁻⁷ sec⁻¹ or 10⁻⁷ sec⁻¹.

A binding molecule, e.g., an antibody or antigen-binding fragment, variant, or derivative disclosed herein can be said to bind a target antigen with an on rate (k(on)) of greater than or equal to 10³ M⁻¹ sec⁻¹, 5×10³ M⁻¹ sec⁻¹, 10⁴ M⁻¹ sec⁻¹, 5×10⁴ M⁻¹ sec⁻¹, 10⁵ M⁻¹ sec⁻¹, 5×10⁵M⁻¹ sec⁻¹, 10⁶M⁻¹ sec⁻¹, or 5×10⁶M⁻¹ sec⁻¹ or 10⁷M⁻¹ sec⁻¹.

A binding molecule, e.g., an antibody or fragment, variant, or derivative thereof is said to competitively inhibit binding of a reference antibody or antigen binding fragment to a given epitope if it preferentially binds to that epitope to the extent that it blocks, to some degree, binding of the reference antibody or antigen binding fragment to the epitope. Competitive inhibition can be determined by any method known in the art, for example, competition ELISA assays. A binding molecule can be said to competitively inhibit binding of the reference antibody or antigen binding fragment to a given epitope by at least 90%, at least 80%, at least 70%, at least 60%, or at least 50%.

As used herein, the term “affinity” refers to a measure of the strength of the binding of an individual epitope with one or more binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of binding domains and an antigen. See, e.g., Harlow at pages 29-34. Avidity is related to both the affinity of individual binding domains in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a between a bivalent monoclonal antibody with a receptor present at a high density on a cell surface would also be of high avidity.

Binding molecules or antigen-binding fragments, variants or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or K_{D} no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹M, 5×10⁻¹⁰ M, 10⁻¹⁰ M 5×10⁻¹¹M, 10⁻¹¹ M, 5×10⁻¹²M, 10⁻¹²M, 5×10⁻¹³M, 10⁻¹³M, 5×10⁻¹⁴M, 10⁻¹⁴M, 5×10⁻¹⁵M, or 10⁻¹⁵M.

Antibody fragments including single-chain antibodies or other binding domains can exist alone or in combination with one or more of the following: hinge region, CH1, CH2, CH3, or CH4 domains, J chain, or secretory component. Also included are antigen-binding fragments that can include any combination of variable region(s) with one or more of a hinge region, CH1, CH2, CH3, or CH4 domains, a J chain, or a secretory component. Binding molecules, e.g., antibodies, or antigen-binding fragments thereof can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al.

As used herein, the term “heavy chain subunit” includes amino acid sequences derived from an immunoglobulin heavy chain, a binding molecule, e.g., an antibody comprising a heavy chain subunit can include at least one of: a VH domain, a CH1 domain, a hinge (e.g., upper, middle, and/or lower hinge region) domain, a CH2 domain, a CH3 domain, a CH4 domain, or a variant or fragment thereof. For example, a binding molecule, e.g., an antibody or fragment, variant, or derivative thereof can include without limitation, in addition to a VH domain, a CH1 domain; a CH1 domain, a hinge, and a CH2 domain; a CH1 domain and a CH3 domain; a CH1 domain, a hinge, and a CH3 domain; or a CH1 domain, a hinge domain, a CH2 domain, and a CH3 domain. In certain aspects a binding molecule, e.g., an antibody or fragment, variant, or derivative thereof can include, in addition to a VH domain, a CH3 domain and a CH4 domain; or a CH3 domain, a CH4 domain, and a J chain. Further, a binding molecule for use in the disclosure can lack certain constant region portions, e.g., all or part of a CH2 domain. It will be understood by one of ordinary skill in the art that these domains (e.g., the heavy chain subunit) can be modified such that they vary in amino acid sequence from the original immunoglobulin molecule.

As used herein, the term “light chain subunit” includes amino acid sequences derived from an immunoglobulin light chain. The light chain subunit includes at least a VL, and can further include a CL (e.g., Cκ or Cλ) domain.

Binding molecules, e.g., antibodies or antigen-binding fragments, variants, or derivatives thereof can be described or specified in terms of the epitope(s) or portion(s) of an antigen that they recognize or specifically bind. The portion of a target antigen that specifically interacts with the antigen binding domain of an antibody is an “epitope,” or an “antigenic determinant.” A target antigen can comprise a single epitope or at least two epitopes, and can include any number of epitopes, depending on the size, conformation, and type of antigen.

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of a typical IgG heavy chain molecule.

As used herein the term “CH2 domain” includes the portion of a heavy chain molecule that extends, e.g., from about amino acid 244 to amino acid 360 of an IgG antibody using conventional numbering schemes (amino acids 244 to 360, Kabat numbering system; and amino acids 231-340, EU numbering system; see Kabat E A et al., op. cit. The CH3 domain extends from the CH2 domain to the C-terminal of the IgG molecule and comprises approximately 108 amino acids. Certain immunoglobulin classes, e.g., IgM, further include a CH4 region.

As used herein, the term “hinge region” includes the portion of a heavy chain molecule that joins the CH1 domain to the CH2 domain in IgG, IgA, and IgD heavy chains. This hinge region comprises approximately 25 amino acids and is flexible, thus allowing the two N-terminal antigen binding regions to move independently.

As used herein the term “disulfide bond” includes the covalent bond formed between two sulfur atoms. The amino acid cysteine comprises a thiol group that can form a disulfide bond or bridge with a second thiol group.

As used herein, the term “chimeric antibody” refers to an antibody in which the immunoreactive region or site is obtained or derived from a first species and the constant region (which can be intact, partial or modified) is obtained from a second species. In some embodiments the target binding region or site will be from a non-human source (e.g. mouse or primate) and the constant region is human.

The terms “multispecific antibody” or “bispecific antibody” refer to an antibody that has binding domains for two or more different epitopes within a single antibody molecule. Other binding molecules in addition to the canonical antibody structure can be constructed with two binding specificities. Epitope binding by bispecific or multispecific antibodies can be simultaneous or sequential. Triomas and hybrid hybridomas are two examples of cell lines that can secrete bispecific antibodies. Bispecific antibodies can also be constructed by recombinant means. (Ströhlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which the variable domain in either the heavy and light chain or both is altered by at least partial replacement of one or more amino acids in either the CDR or framework regions. In certain aspects entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain aspects not all of the CDRs are replaced with the complete CDRs from the donor variable region and yet the antigen binding capacity of the donor can still be transferred to the recipient variable domains. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

As used herein, the term “cross-linked” refers to joining together of two or more molecules by a third molecule. For example, a bivalent antibody with two binding domains that specifically bind to the same antigen can “cross-link” two copies of that antigen, e.g., as they are expressed on a cell. Signal transduction via TNFSFRs typically requires that three or more receptor monomers be brought into close proximity on the surface of a cell. This is naturally accomplished by engagement of the receptor monomers via a homotrimeric ligand. A typical bivalent IgG antibody is capable of engaging only two TNFSFR monomers on the surface of a cell, and thus such bivalent antibodies must be themselves cross-linked to effectively activate the receptor. Such cross-linking can be accomplished, e.g., with a secondary antibody which binds to the Fc region of bivalent antibody, or by Fc gamma receptors (FcγR). A “secondary cross-linking moiety” as used herein can be any substance capable of cross-linking binding molecules, e.g., binding molecules specific for a TNFSFR. A dimeric, pentameric, or hexameric binding molecule as provided herein comprises up to four, ten, or twelve identical antigen-binding domains in a single covalent molecule. Each antigen-binding domain can engage a TNFSFR monomer, clustering the monomers in close proximity. Thus, a dimeric, pentameric, or hexameric binding molecule as provided herein can, for example specifically bind to and cross-link at least three, e.g., four, ten, or twelve TNFSFRs simultaneously, thereby activating signal transduction in the absence of a secondary cross-linking moiety.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly a portion of a polypeptide that is “carboxy-terminal” or “C-terminal” to another portion of a polypeptide is that portion that comes later in the sequential polypeptide chain. For example in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

Terms such as “treating” or “treatment” or “to treat” or “alleviating” or “to alleviate” refer to therapeutic measures that cure, slow down, lessen symptoms of, and/or halt or slow the progression of an existing diagnosed pathologic condition or disorder. Terms such as “prevent,” “prevention,” “avoid,” “deterrence” and the like refer to prophylactic or preventative measures that prevent the development of an undiagnosed targeted pathologic condition or disorder. Thus, “those in need of treatment” can include those already with the disorder; those prone to have the disorder; and those in whom the disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, phrases such as “a subject that would benefit from therapy” and “an animal in need of treatment” refers to a subset of subjects, from amongst all prospective subjects, which would benefit from administration of a given therapeutic agent, e.g., a binding molecule such as an antibody, comprising one or more antigen binding domains. Such binding molecules, e.g., antibodies, can be used, e.g., for a diagnostic procedures and/or for treatment or prevention of a disease.

IgM Binding Molecules

IgM is the first immunoglobulin produced by B cells in response to stimulation by antigen, and is present at around 1.5 mg/ml in serum with a half-life of 5 days. IgM is a pentameric or hexameric molecule. An IgM binding unit includes two light and two heavy chains. While IgG contains three heavy chain constant domains (CH1, CH2 and CH3), the heavy (μ) chain of IgM additionally contains a fourth constant domain (CH4), that includes a C-terminal “tailpiece.” The human IgM constant region typically comprises the amino acid sequence SEQ ID NO: 1. The human Cμ1 region ranges from about amino acid 5 to about amino acid 102 of SEQ ID NO: 1; the human Cμ2 region ranges from about amino acid 114 to about amino acid 205 of SEQ ID NO: 1, the human Cμ3 region ranges from about amino acid 224 to about amino acid 319 of SEQ ID NO: 1, the Cμ4 region ranges from about amino acid 329 to about amino acid 430 of SEQ ID NO: 1, and the tailpiece ranges from about amino acid 431 to about amino acid 453 of SEQ ID NO: 1. SEQ ID NO: 1 is presented below:

GSASAPTLFPLVSCENSPSDTSSVAVGCLAQDFLPDSI
TLSWKYKNNSDISSTRGFPSVLRGGKYAATSQVLLPS
KDVMQGTDEHVVCKVQHPNGNKEKNVPLPVIAELPP
KVSVFVPPRDGFFGNPRKSKLICQATGFSPRQIQVSWL
REGKQVGSGVTTDQVQAEAKESGPTTYKVTSTLTIKE
SDWLGQSMFTCRVDHRGLTFQQNASSMCVPDQDTAI
RVFAIPPSFASIFLTKSTKLTCLVTDLTTYDSVTISWTR
QNGEAVKTHTNISESHPNATFSAVGEASICEDDWNSG
ERFTCTVTHTDLPSPLKQTISRPKGVALHRPDVYLLPP
AREQLNLRESATITCLVTGFSPADVFVQWMQRGQPLS
PEKYVTSAPMPEPQAPGRYFAHSILTVSEEEWNTGET
YTCVAHEALPNRVTERTVDKSTGKPTLYNVSLVMSD
TAGTCY

Five IgM binding units can form a complex with an additional small polypeptide chain (the J chain) to form an IgM antibody. The human J chain comprises the amino acid sequence SEQ ID NO: 2. Without the J chain, IgM binding units typically assemble into a hexamer. While not wishing to be bound by theory, the assembly of IgM binding units into a pentameric or hexameric binding molecule is thought to involve the Cμ3 and Cμ4 domains. Accordingly, a pentameric or hexameric binding molecule provided in this disclosure typically includes IgM constant regions that include at least the Cμ3 and Cμ4 domains. SEQ ID NO: 2 is presented below:

MKNHLLFWGVLAVFIKAVHVKAQEDERIVLVDNKC
KCARITSRIIRSSEDPNEDIVERNIRIIVPLNNRENISDPT
SPLRTRFVYHLSDLCKKCDPTEVELDNQIVTATQSNIC
DEDSATETCYTYDRNKCYTAVVPLVYGGETKMVET
ALTPDACYPD

An IgM heavy chain constant region can additionally include a Cμ2 domain or a fragment thereof, a Cμ1 domain or a fragment thereof, and/or other IgM heavy chain domains. In certain aspects, a binding molecule as provided herein can include a complete IgM heavy (μ) chain constant domain, e.g., SEQ ID NO: 1, or a variant, derivative, or analog thereof.

Agonistic Pentameric or Hexameric GITR Binding Molecules

This disclosure provides a pentameric or hexameric binding molecule, i.e., a binding molecule with five or six “binding units” as defined herein, that can specifically bind to three or more, e.g., four or more, e.g., five, six, seven, eight, nine, ten, eleven, or twelve GITR monomers, e.g., murine and/or human GITR monomers. In certain aspects, where GITR is expressed on a cell, e.g., a T cell, e.g., a Treg or an activated effector CTL, a pentameric or hexameric binding molecule as provided herein can sufficiently engage multiple, e.g., three or more GITR monomers on the cell to trigger a signal transduction pathway in the absence of a secondary cross-linking moiety, thereby inducing anti-tumor immunity. A binding molecule as provided herein can possess improved binding characteristics or biological activity as compared to a binding molecule composed of a single binding unit, e.g., a bivalent IgG antibody. For example, a pentameric or hexameric binding molecule can more efficiently cross-link multiple, e.g., three or more GITR receptors on the surface of a cell, and/or can effectively cross-link multiple, e.g., three or more GITR receptors on the surface of a cell in the absence of a secondary cross-linking moiety such as, but not limited to an FcγR, thereby facilitating anti-tumor immunity.

A binding molecule as provided herein can likewise possess distinctive characteristics compared to multivalent binding molecules composed of synthetic or chimeric structures. For example, use of human IgM constant regions can afford reduced immunogenicity and thus increased safety relative to a binding molecule containing chimeric constant regions or synthetic structures. Moreover, an IgM-based binding molecule can consistently form hexameric or pentameric oligomers resulting in a more homogeneous expression product. Superior complement fixation can also be an advantageous effector function of IgM-based binding molecules.

In certain aspects, the disclosure provides a pentameric or hexameric binding molecule comprising five or six bivalent binding units, respectively, where each binding unit includes two IgM heavy chain constant regions or fragments or variants thereof. In certain aspects, the two IgM heavy chain constant regions are human heavy chain constant regions.

Where the binding molecule provided herein is pentameric, the binding molecule can further comprise a J chain, or fragment thereof, or variant thereof. In certain aspects the J chain can be modified, as discussed elsewhere herein.

An IgM heavy chain constant region can include one or more of a Cμ1 domain or fragment or variant thereof, a Cμ2 domain or fragment or variant thereof, a Cμ3 domain or fragment or variant thereof, and/or a Cμ4 domain or fragment or variant thereof, provided that the constant region can serve a desired function in the binding molecule, e.g., associate with second IgM constant region to form a binding domain, or associate with other binding units to form a hexamer or a pentamer. In certain aspects the two IgM heavy chain constant regions or fragments or variants thereof within an individual binding unit each comprise a Cμ3 domain or fragment or variant thereof, a Cμ4 domain or fragment or variant thereof, a tailpiece (TP) or fragment or variant thereof, or any combination of a Cμ3 domain a Cμ domain, and a TP or fragment or variant thereof. In certain aspects the two IgM heavy chain constant regions or fragments or variants thereof within an individual binding unit each further comprise a Cμ2 domain or fragment or variant thereof, a Cμ1 domain or fragment or variant thereof, or a Cμ1 domain or fragment or variant thereof and a Cμ2 domain or fragment or variant thereof.

In certain aspects each of the two IgM heavy chain constant regions in a given binding unit is associated with an antigen-binding domain, for example an Fv portion of an antibody, e.g., a VH and a VL of a human or murine antibody, where the VL can be associated with a light chain constant region. In a binding molecule as provided herein at least three antigen-binding domains of the binding molecule are GITR binding domains that can specifically and agonistically bind to GITR, e.g., human and/or murine GITR.

IgA Binding Molecules

IgA plays a critical role in mucosal immunity, and comprises about 15% of total immunoglobulin produced. IgA is a monomeric or dimeric molecule. An IgA binding unit includes two light and two heavy chains. IgA contains three heavy chain constant domains (Cα1, Cα2 and Cα3), and includes a C-terminal “tailpiece.” Human IgA has two subtypes, IgA1 and IgA2. The human IgA1 constant region typically comprises the amino acid sequence SEQ ID NO: 3. The human Cα1 region ranges from about amino acid 6 to about amino acid 98 of SEQ ID NO: 3; the human Cα2 region ranges from about amino acid 125 to about amino acid 220 of SEQ ID NO: 3, the human Cα3 region ranges from about amino acid 228 to about amino acid 330 of SEQ ID NO: 3, and the tailpiece ranges from about amino acid 331 to about amino acid 352 of SEQ ID NO: 3. The human IgA2 constant region typically comprises the amino acid sequence SEQ ID NO: 4. The human Cα1 region ranges from about amino acid 6 to about amino acid 98 of SEQ ID NO: 4; the human Cα2 region ranges from about amino acid 112 to about amino acid 207 of SEQ ID NO: 4, the human Cα3 region ranges from about amino acid 215 to about amino acid 317 of SEQ ID NO: 4, and the tailpiece ranges from about amino acid 318 to about amino acid 340 of SEQ ID NO: 4. SEQ ID NOS: 3 and 4 are presented below:

SEQ ID NO: 3
ASPTSPKVFPLSLCSTQPDGNVVIACLVQGFFPQEPLS
VTWSESGQGVTARNFPPSQDASGDLYTTSSQLTLPAT
QCLAGKSVTCHVKHYTNPSQDVTVPCPVPSTPPTPSP
STPPTPSPSCCHPRLSLHRPALEDLLLGSEANLTCTLTG
LRDASGVTFTWTPSSGKSAVQGPPERDLCGCYSVSSV
LPGCAEPWNHGKTFTCTAAYPESKTPLTATLSKSGNT
FRPEVHLLPPPSEELALNELVTLTCLARGFSPKDVLVR
WLQGSQELPREKYLTWASRQEPSQGTTTFAVTSILRV
AAEDWKKGDTFSCMVGHEALPLAFTQKTIDRLAGKP
THVNVSVVMAEVDGTCY
SEQ ID NO: 4
ASPTSPKVFPLSLDSTPQDGNVVVACLVQGFFPQEPLS
VTWSESGQNVTARNFPPSQDASGDLYTTSSQLTLPAT
QCPDGKSVTCHVKHYTNPSQDVTVPCPVPPPPPCCHP
RLSLHRPALEDLLLGSEANLTCTLTGLRDASGATFTW
TPSSGKSAVQGPPERDLCGCYSVSSVLPGCAQPWNH
GETFTCTAAHPELKTPLTANITKSGNTFRPEVHLLPPP
SEELALNELVTLTCLARGFSPKDVLVRWLQGSQELPR
EKYLTWASRQEPSQGTTTFAVTSILRVAAEDWKKGD
TFSCMVGHEALPLAFTQKTIDRMAGKPTHVNVSVVM
AEVDGTCY

Two IgA binding units can form a complex with two additional polypeptide chains, the J chain (SEQ ID NO: 2) and the secretory component (precursor, SEQ ID NO: 5, mature, SEQ ID NO: 6) to form a secretory IgA (sIgA) antibody. While not wishing to be bound by theory, the assembly of IgA binding units into a dimeric sIgA binding molecule is thought to involve the Cα3 and tailpiece domains. Accordingly, a dimeric sIgA binding molecule provided in this disclosure typically includes IgA constant regions that include at least the Cα3 and tailpiece domains. SEQ ID NO: 5 and SEQ ID NO: 6 are presented below:

SEQ ID NO: 5:
MLLFVLTCLLAVFPAISTKSPIFGPEEVNSVEGNSVSIT
CYYPPTSVNRHTRKYWCRQGARGGCITLISSEGYVSS
KYAGRANLTNFPENGTFVVNIAQLSQDDSGRYKCGL
GINSRGLSFDVSLEVSQGPGLLNDTKVYTVDLGRTVT
INCPFKTENAQKRKSLYKQIGLYPVLVIDSSGYVNPN
YTGRIRLDIQGTGQLLFSVVINQLRLSDAGQYLCQAG
DDSNSNKKNADLQVLKPEPELVYEDLRGSVTFHCAL
GPEVANVAKFLCRQSSGENCDVVVNTLGKRAPAFEG
RILLNPQDKDGSFSVVITGLRKEDAGRYLCGAHSDGQ
LQEGSPIQAWQLFVNEESTIPRSPTVVKGVAGGSVAV
LCPYNRKESKSIKYWCLWEGAQNGRCPLLVDSEGWV
KAQYEGRLSLLEEPGNGTFTVILNQLTSRDAGFYWCL
TNGDTLWRTTVEIKIIEGEPNLKVPGNVTAVLGETLK
VPCHFPCKFSSYEKYWCKWNNTGCQALPSQDEGPSK
AFVNCDENSRLVSLTLNLVTRADEGWYWCGVKQGH
FYGETAAVYVAVEERKAAGSRDVSLAKADAAPDEK
VLDSGFREIENKAIQDPRLFAEEKAVADTRDQADGSR
ASVDSGSSEEQGGSSRALVSTLVPLGLVLAVGAVAV
GVARARHRKNVDRVSIRSYRTDISMSDFENSREFGAN
DNMGASSITQETSLGGKEEFVATTESTTETKEPKKAK
RSSKEEAEMAYKDFLLQSSTVAAEAQDGPQEA
SEQ ID NO: 6:
KSPIFGPEEVNSVEGNSVSITCYYPPTSVNRHTRKYWC
RQGARGGCITLISSEGYVSSKYAGRANLTNFPENGTF
VVNIAQLSQDDSGRYKCGLGINSRGLSFDVSLEVSQG
PGLLNDTKVYTVDLGRTVTINCPFKTENAQKRKSLYK
QIGLYPVLVIDSSGYVNPNYTGRIRLDIQGTGQLLFSV
VINQLRLSDAGQYLCQAGDDSNSNKKNADLQVLKPE
PELVYEDLRGSVTFHCALGPEVANVAKFLCRQSSGEN
CDVVVNTLGKRAPAFEGRILLNPQDKDGSFSVVITGL
RKEDAGRYLCGAHSDGQLQEGSPIQAWQLFVNEESTI
PRSPTVVKGVAGGSVAVLCPYNRKESKSIKYWCLWE
GAQNGRCPLLVDSEGWVKAQYEGRLSLLEEPGNGTF
TVILNQLTSRDAGFYWCLTNGDTLWRTTVEIKIIEGEP
NLKVPGNVTAVLGETLKVPCHFPCKFSSYEKYVVCKW
NNTGCQALPSQDEGPSKAFVNCDENSRLVSLTLNLVT
RADEGWYWCGVKQGHFYGETAAVYVAVEERKAAG
SRDVSLAKADAAPDEKVLDSGFREIENKAIQDPR

An IgA heavy chain constant region can additionally include a Cα2 domain or a fragment thereof, a Cα1 domain or a fragment thereof, and/or other IgA heavy chain domains. In certain aspects, a binding molecule as provided herein can include a complete IgA heavy (α) chain constant domain (e.g., SEQ ID NO: 3 or SEQ ID NO: 4), or a variant, derivative, or analog thereof.

Agonistic Dimeric GITR Binding Molecules

This disclosure provides a dimeric binding molecule, e.g., a binding molecule with two IgA “binding units” as defined herein that can specifically bind to three or more or up to four GITR monomers, e.g., human or murine GITR monomers. In certain aspects, where GITR is expressed on a cell, e.g., a T cell, e.g., a Treg or an activated effector CTL, contacting multiple GITR receptors on the cell with a binding molecule as provided herein can trigger a signal transduction pathway in the absence of a secondary cross-linking moiety, thereby inducing anti-tumor immunity. A dimeric binding molecule as provided herein can possess improved binding characteristics or biological activity as compared to a binding molecule composed of a single binding unit, e.g., a bivalent IgG antibody. For example, an IgA binding molecule can more efficiently cross-link multiple, e.g., three or more GITR receptors on the surface of a cell, and/or can effectively cross-link multiple, e.g., three or more GITR receptors on the surface of a cell in the absence of a secondary cross-linking moiety such as, but not limited to a FcγR, thereby facilitating anti-tumor immunity. Moreover, an IgA binding molecule can reach mucosal sites providing greater tissue distribution for the binding molecules provided herein. Use of an IgA-based binding molecule can allow, for example, greater tissue distribution for a binding molecule provided herein. Mucosal distribution could be beneficial to reach the tumor microenvironment of certain cancers, e.g., lung cancer, ovarian cancer, colorectal cancer, or squamous cell carcinoma. Likewise, a dimeric binding molecule as provided herein can possess binding characteristics or biological activity that can be distinguished from a binding molecule comprising five or six binding units, e.g., a hexameric or pentameric IgM antibody. For example, a dimeric binding molecule would be smaller, and could, for example, achieve better tissue penetration in solid tumors.

In certain aspects, the disclosure provides a dimeric binding molecule comprising two bivalent binding units, where each binding unit includes two IgA heavy chain constant regions or fragments or variants thereof. In certain aspects, the two IgA heavy chain constant regions are human heavy chain constant regions.

A dimeric IgA binding molecule as provided herein can further comprise a J chain, or fragment thereof, or variant thereof, e.g., a modified J chain as disclosed elsewhere herein. A dimeric IgA binding molecule as provided herein can further comprise a secretory component, or fragment thereof, or variant thereof.

An IgA heavy chain constant region can include one or more of a Cα1 domain, a Cα2 domain, and/or a Cα3 domain, provided that the constant region can serve a desired function in the binding molecule, e.g., associate with a light chain constant region to facilitate formation of an antigen binding domain, or associate with another IgA binding unit to form a dimeric binding molecule. In certain aspects the two IgA heavy chain constant regions or fragments or variants thereof within an individual binding unit each comprise a Cα3 domain or fragment or variant thereof, a tailpiece (TP) or fragment or variant thereof, or any combination of a Cα3 domain, a TP, or fragment or variant thereof. In certain aspects the two IgA heavy chain constant regions or fragments thereof within an individual binding unit each further comprise a Cα2 domain or fragment or variant thereof, a Cα1 domain or fragment or variant thereof, or a Cα1 domain or fragment or variant thereof and a Cα2 domain or fragment or variant thereof.

In certain aspects each of the two IgA heavy chain constant regions in a given binding unit is associated with an antigen binding domain, for example an Fv portion of an antibody, e.g., a VH and a VL of a human or murine antibody, where the VL can be associated with a light chain constant region. In a binding molecule as provided herein at least three antigen-binding domains of the binding molecule specifically and agonistically bind to GITR, e.g., human and/or murine GITR.

Multispecific Dimeric, Pentameric or Hexameric GITR Agonist Binding Molecules

A multi-specific, e.g., bispecific dimeric GITR agonist binding molecule as provided herein can be based on the dimeric form of an IgA antibody, in which two pairs of IgA heavy chain sequences can be present with or without associated light chain sequences. For example, a bispecific dimeric GITR agonist binding molecule as provided herein can be composed of two IgA (IgA1 or IgA2) dimers, including a J chain, e.g., a modified J chain as provided elsewhere herein.

A multi-specific, e.g., bispecific dimeric GITR agonist binding molecule as provided herein can include mono- and bispecific binding units as long as the molecule as a whole has at least two binding specificities, e.g., at least two non-identical antigen-binding domains, e.g., different epitopes of GITR, epitopes from other TNFSFR molecules, or heterologous antigens.

Thus, in one embodiment, a multi-specific, e.g., bispecific dimeric binding molecule as provided herein can include two monospecific binding units (AA, BB), each having bivalent binding specificity to a different binding target. In another embodiment, a multi-specific, e.g., bispecific dimeric binding molecule as provided herein can include two bispecific binding units, each binding unit binding to the same two binding targets (AB, AB) to form a bispecific dimeric binding molecule. In a further embodiment, one binding unit present in a multi-specific dimeric binding molecule as provided herein is monospecific (AA) while the other binding units are bispecific (BC), resulting in a multispecific binding molecule with three (A, B, C) binding specificities. In a further embodiment, each binding unit is bispecific, but one specificity is overlapping (e.g. AB, AC), resulting in a multispecific binding molecule with three (A, B, C) binding specificities. Other combinations, e.g., with four non-identical antigen binding domains (A, B, C, D) can be readily made based on this disclosure.

A multi-specific, e.g., bispecific pentameric or hexameric GITR agonist binding molecule as provided herein can be based on the pentameric or hexameric forms of an IgM antibody, in which five or six pairs of IgM heavy chain sequences can be present with or without associated light chain sequences. For example, a bispecific hexameric or pentameric GITR agonist binding molecule as provided herein can be composed of five IgM dimers, including a J chain, e.g., a modified J chain as provided elsewhere herein, or six IgM dimers.

A multi-specific, e.g., bispecific pentameric or hexameric GITR agonist binding molecule as provided herein can include mono- and bispecific binding units as long as the molecule as a whole has at least two binding specificities, e.g., at least two non-identical antigen-binding domains, e.g., different epitopes of GITR, epitopes from other TNFSFR molecules, or heterologous antigens.

As discussed above for multispecific dimeric binding molecules, each of the five or six binding units can independently be monospecific or bispecific (e.g., AA, BB, CC, etc.) or one or more binding units can be bispecific (e.g., AB, AB, AC, CD, etc.). Thus, a multi-specific, e.g., bispecific pentameric or hexameric binding molecule as provided herein can include at least two independent antigen binding domains, and up to twelve different, independent antigen binding domains.

Modified J Chains

In certain aspects, the J chain of dimeric or pentameric binding molecules as provided herein can be modified, e.g., by introduction of a heterologous moiety, or two or more heterologous moieties, without interfering with the ability of the IgM or IgA binding molecule to assemble and bind to its binding target(s). See PCT Application No. PCT/US2015/024149 (Publication WO 2015/153912), PCT Application No. PCT/US2016/055053 (Publication WO 2017/059387), and PCT Application No. PCT/US2016/055041 (Publication WO 2017/059380) each of which is incorporated herein by reference in its entirety. Accordingly, dimeric or pentameric binding molecules as provided herein, including multispecific dimeric or pentameric binding molecules as described elsewhere herein, can comprise a modified J chain or functional fragment thereof comprising a heterologous moiety introduced into the J chain or fragment thereof. In certain aspects heterologous moiety can be a peptide or polypeptide sequence fused in frame to the J chain or chemically conjugated to the J chain. In certain aspects the heterologous moiety can be a chemical moiety conjugated to the J chain. Heterologous moieties to be attached to a J chain can include, without limitation, a binding moiety, e.g., an antibody or antigen binding fragment thereof, e.g., a single chain Fv (ScFv) molecule, a stabilizing peptide that can increase the half-life of the dimeric or pentameric binding molecule, or a chemical moiety such as a polymer or a cytotoxin.

In some embodiments, a modified J chain can comprise an antigen binding domain that can include, without limitation, a polypeptide (including small peptides) capable of specifically binding to a target antigen. In certain aspects, an antigen binding domain associated with a modified J chain can be an antibody or an antigen-binding fragment thereof, as described elsewhere herein. In certain aspects the antigen binding domain can be a scFv binding domain or a single-chain binding domain derived, e.g., from a camelid or condricthoid antibody. The antigen binding domain can be introduced into the J chain at any location that allows the binding of the antigen binding domain to its binding target without interfering with J chain function or the function of an associated IgM or IgA antibody. Insertion locations include, but are not limited to: at or near the C-terminus, at or near the N-terminus or at an internal location that, based on the three-dimensional structure of the J chain, is accessible. In certain aspects, the antigen binding domain can be introduced into the human J chain of SEQ ID NO: 2 between cysteine residues 92 and 101 of SEQ ID NO: 2. In a further aspect, the antigen binding domain can be introduced into the human J chain of SEQ ID NO: 2 at or near a glycosylation site. In a further aspect, the antigen binding domain can be introduced into the human J chain of SEQ ID NO: 2 within about 10 amino acid residues from the C-terminus.

GITR Binding Domains

A GITR agonist binding molecule as provided herein can be dimeric, pentameric, or hexameric, comprising two, five, or six bivalent binding units, respectively. The binding units can be full length or variants or fragments thereof that retain binding function.

Each binding unit comprises two IgA or IgM heavy chain constant regions or fragments thereof, each associated with an antigen-binding domain. As noted above, an antigen binding domain is a region of a binding molecule that is necessary and sufficient to specifically bind to an epitope. A “binding molecule” as described herein can include one, two, three, four, five, six, seven, eight, nine, ten, eleven, twelve or more “antigen binding domains.”

A dimeric, pentameric, or hexameric binding molecule as provided herein can include at least three antigen-binding domains that specifically and agonistically bind to GITR, e.g., human and/or murine GITR. As noted above, dimeric, pentameric, or hexameric GITR agonist binding molecules as provided herein can specifically bind to and engage multiple, e.g., three or more GITR monomers. In certain aspects, where GITR is expressed on a cell, e.g., a T cell, e.g., a Treg or an activated effector CTL, contacting multiple, e.g., three or more GITR receptors on the cell with a binding molecule as provided herein can trigger a signal transduction pathway thereby inducing anti-tumor immunity. A signal transduction pathway can be triggered when multiple receptor proteins are bound together, causing cross-linking of the receptor molecules such that a signal is transmitted across the cell membrane into the cytosol of the GITR-expressing cell.

A dimeric, pentameric, or hexameric binding molecule as provided herein can cross-link at least three GITR monomers expressed on the surface of a cell. Due to its dimeric, pentameric, or hexameric nature, a GITR agonist binding molecule as provided herein can cross-link as many as three, four, five, six, seven, eight, nine, ten, eleven, or twelve GITR monomers. The GITR monomers are necessarily spatially brought into proximity of each other, often into a lipid raft, which can contribute to their cross-linking and further enhance activation. When all five or all six of the bivalent binding units of a pentameric or hexameric GITR agonist binding molecule as provided herein bind to up to ten or twelve GITR monomers on a single cell, cross-linking and activation of the receptors can occur with high efficiency.

Because each of the binding units is bivalent, each binding molecule can bind to as many as 4 (for dimeric binding molecules), 10 (for pentameric binding molecules), or 12 (for hexameric binding molecules) GITR monomers.

Upon activation of the receptors by the binding of a dimeric, pentameric, or hexameric binding molecule as provided herein, the cell, e.g., a T cell, e.g., a Treg or an activated effector CTL, can be activated thereby inducing anti-tumor immunity through, e.g., CTL activation (proliferation, tumor cell killing) or interference with Treg immune suppression.

In certain aspects, a dimeric, pentameric, or hexameric binding molecule as provided herein can induce signal transduction in a GITR-expressing cell at a higher potency than an equivalent amount of a bivalent IgG antibody or fragment thereof, which also specifically binds to and agonizes the same GITR epitope. Not wishing to be bound by theory, because a provided binding molecule is dimeric, pentameric, or hexameric, and because each binding unit is bivalent, such a binding molecule can induce receptor-mediated functions previously characterized for GITR at a higher potency than any single binding unit alone, such as an equivalent IgG binding unit. IgG binding units are bivalent, containing two binding sites, but as previous clinical studies have shown, binding of two GITR monomers with a single IgG molecule can be ineffective without addition of other components, such as cross-linkers, etc.

By “potency” or “improved binding characteristics” is meant the least amount of a given binding molecule necessary to achieve a given biological result, e.g., activation of 20%, 50%, or 90% of GITR signal transduction activity in a given assay, e.g., a T cell signaling assay, a T cell proliferation assay, a T cell activation and cytokine secretion assay, a cytotoxicity assay, or other assay as provided in the examples below.

Because a binding molecule as provided herein is dimeric, pentameric, or hexameric, it can contain as many as 4, 10, or 12, respectively, GITR-specific antigen-binding domains. Each of the antigen-binding domains can specifically bind to a GITR monomer, gathering the monomers together to provide agonistic activity. Further, different antigen-binding domains can be specific for two or more particular GITR epitopes.

Thus, a single dimeric, pentameric, or hexameric binding molecule can: a) simultaneously bind a single epitope on many GITR monomers, or b) bind different epitopes on a single GITR monomer, or c) can bind different epitopes on different TNFSFR proteins in addition to GITR. In embodiment a), a GITR agonist binding molecule as provided herein can bind multiple GITR monomers, thereby forming a raft of such monomers in a single location, increasing the likelihood that the receptor will be activated. In other embodiments, such as embodiment c), a dimeric, pentameric, or hexameric binding molecule as provided herein can be used to contact GITR as well as other TNFSFR proteins, e.g., OX40 and/or CD137/4-1BB, thereby activating more than one pathway through the various targeted receptors, to achieve a desired biological response in the cells. In these embodiments, a GITR agonist binding molecule as provided herein can contact and agonize such receptors all on one single cell, or across multiple cells.

Thus, a dimeric, pentameric, or hexameric binding molecule as provided herein can comprise three, four, five, six, seven, eight, nine, ten, or in the case of the hexameric binding molecules, as many as eleven, or twelve antigen-binding domains that specifically and agonistically bind to GITR, and optionally one or more additional TNFSFR proteins expressed on the surface of one or more cells, thereby inducing the intended or desired biological response in the cell(s).

The binding units of a dimeric, pentameric, or hexameric binding molecule as provided herein can be human, humanized, or chimeric immunoglobulin binding units. Methods of humanizing immunoglobulin sequences are well known in the art. Thus, the nucleotide sequences encoding a dimeric, pentameric, or hexameric binding molecule polypeptide can be directly from human sequences, or can be humanized or chimeric, i.e., encoded by sequences from multiple different species.

The cells which express GITR can be any animal cell. For instance, in one embodiment, the cell is a human cell, e.g., a human T cell, e.g., a human CTL. In other aspects, the cell can be, e.g., any one or more of primate, rodent, canine, equine, etc., cells.

A dimeric, pentameric, or hexameric binding molecule as provided herein can be genetically engineered such that its antigen-binding domains are encoded by sequences known to specifically bind GITR, e.g., human and/or murine GITR. Many groups have published sequences of variable regions of monoclonal antibodies, most of the IgG isotype, which are characterized and are known to specifically bind to GITR. Non-limiting immunoglobulin variable domain sequences that are known to specifically bind to GITR are provided in Table 2. Other monoclonal antibody sequences specific for GITR have been published. One of skill in the art is capable of engineering these published sequences into immunoglobulin structures, such as an IgG, IgA, IgM structure, or biologically active or functional fragments thereof (such as scFv fragments and the like, as discussed above). Methods for genetically engineering cloned variable regions into immunoglobulin domains, and expressing and purifying such constructs are published and within the capability of one skilled in the art.

Thus, in certain aspects, a GITR binding domain as provided herein comprises six immunoglobulin complementarity determining regions HCDR1, HCDR2, HCDR3, LCDR1, LCDR2, and LCDR3, or the six immunoglobulin complementarity determining regions with one, two, three, four, or five single amino acid substitutions in one or more of the CDRs, of an anti-GITR mAb comprising the mature VH and VL amino acid sequences comprising or contained within SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 15 and SEQ ID NO: 17; SEQ ID NO: 18 and SEQ ID NO: 19; SEQ ID NO: 20 and SEQ ID NO: 21; SEQ ID NO: 22 and SEQ ID NO: 23; SEQ ID NO: 22 and SEQ ID NO: 24; SEQ ID NO: 25 and SEQ ID NO: 26; SEQ ID NO: 27 and SEQ ID NO: 28; SEQ ID NO: 27 and SEQ ID NO: 29; SEQ ID NO: 30 and SEQ ID NO: 31; SEQ ID NO: 32 and SEQ ID NO: 33; SEQ ID NO: 32 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 57 and SEQ ID NO: 58; SEQ ID NO: 59 and SEQ ID NO: 60; SEQ ID NO: 61 and SEQ ID NO: 62; SEQ ID NO: 63 and SEQ ID NO: 64; SEQ ID NO: 65 and SEQ ID NO: 66; SEQ ID NO: 67 and SEQ ID NO: 68; SEQ ID NO: 69 and SEQ ID NO: 68; SEQ ID NO: 70 and SEQ ID NO: 71; SEQ ID NO: 72 and SEQ ID NO: 71; SEQ ID NO: 73 and SEQ ID NO: 74; SEQ ID NO: 75 and SEQ ID NO: 76; SEQ ID NO: 77 and SEQ ID NO: 78; SEQ ID NO: 79 and SEQ ID NO: 80; SEQ ID NO: 81 and SEQ ID NO: 82; SEQ ID NO: 83 and SEQ ID NO: 84; SEQ ID NO: 85 and SEQ ID NO: 86; SEQ ID NO: 87 and SEQ ID NO: 88; SEQ ID NO: 89 and SEQ ID NO: 90; SEQ ID NO: 91 and SEQ ID NO: 92; SEQ ID NO: 93 and SEQ ID NO: 94; SEQ ID NO: 95 and SEQ ID NO: 96; SEQ ID NO: 97 and SEQ ID NO: 98; SEQ ID NO: 99 and SEQ ID NO: 98; SEQ ID NO: 100 and SEQ ID NO: 101; SEQ ID NO: 102 and SEQ ID NO: 103; SEQ ID NO: 104 and SEQ ID NO: 101; SEQ ID NO: 105 and SEQ ID NO: 101; SEQ ID NO: 106 and SEQ ID NO: 101; SEQ ID NO: 107 and SEQ ID NO: 101; SEQ ID NO: 108 and SEQ ID NO: 101; or SEQ ID NO: 109 and SEQ ID NO: 110, respectively.

TABLE 2
Anti-GITR Agonist Antibody VH and VL Sequences

| | VH SEQ | | VL SEQ | |
|---|---|---|---|---|
| Reference | ID NO | VH | ID NO | VL |
| U.S. Pat. No. | 9 | QVQLVESGGGVVQPGRSLRLSCAAS | 10 | AIQLTQSPSSLSASVGDRVTITCRASQGISS |
| 9,228,016B2 | | GFTFSSYGMHWVRQAPGKGLEWVA | | ALAWYQQKPGKAPKLLIYDASSLESGVPS |
| | | VIWYEGSNKYYADSVKGRFTISRDNS | | RFSGSGSGTDFTLTISSLQPEDFATYYCQQ |
| | | KNTLYLQMNSLRAEDTAVYYCARG | | FNSYPYTFGQGTKLEIK |
| | | GSMVRGDYYYGMDVWGQGTTVTV | | |
| | | SS | | |
| U.S. Pat. No. | 11 | QVQLVESGGGVVQPGRSLRLSCAAS | 12 | DIQMTQSPSSLSASVGDRVTITCRASQGISS |
| 9,228,016B2 | | GFTFSSYGFHWVRQAPGKGLEWVA | | WLAWYQQKPEKAPKSLIYAASSLQSGVPS |
| | | VIWYAGSNKFYADSVKGRFTISRDNS | | RFSGSGSGTDFTLTISSLQPEDFATYYCQQ |
| | | KNTLYLQMNSLRAEDTAVYYCARG | | YNSYPYTFGQGTKLEIK |
| | | GQLDYYYYYVMDVWGQGTTVTVSS | | |
| U.S. Pat. No. | 13 | QVQLVESGGGVVQPGRSLRLSCAAS | 14 | DIQMTQSPSSLSASVGDRVTITCRASQGISS |
| 9,228,016B2 | | GFTFSSYGMHWVRQAPGKGLEWVA | | WLAWYQQKPEKAPKSLIYAASSLQSGVPS |
| | | VIWYAGSNKYYADSVKGRFTISRDN | | RFSGSGSGTDFTLTISSLQPEDFATYYCQQ |
| | | SKNTLYLQMNSLRAEDTAVYYCARG | | YNSYPYTFGQGTKLEIK |
| | | GRIAVAFYYSMDVWGQGTTVTVSS | | |
| U.S. Pat. No. | 15 | QVQLQQWGAGLLKPSETLSLTCAVY | 16 | DIQMTQSPSSLSASVGDRVTITCRASQGISS |
| 9,228,016B2 | | GGSFSGYYWTWIRQPPGKGLEWIGKI | | WLAWYQQKPEKAPKSLIYAASSLQSGVPS |
| | | NHSGNTNYNPSLKSRVTISVDTSKNQ | | RFSGSGSGTDFTLTISSLQPEDFATYYCQQ |
| | | FSLKLSSVTAADTAVYYCARLGAFD | | YNSYPYTFGQGTKLEIK |
| | | AFDIWGQGTMVTVSS | | |
| U.S. Pat. No. | 15 | QVQLQQWGAGLLKPSETLSLTCAVY | 17 | EIVLTQSPATLSLSPGERATLSCRASQGVSS |
| 9,228,016B2 | | GGSFSGYYWTWIRQPPGKGLEWIGKI | | YLAWYQQKPGQAPRLLIYDASNRATGIPA |
| | | NHSGNTNYNPSLKSRVTISVDTSKNQ | | RFSGSGPGTDFTLTISSLEPEDFAVYYCQQ |
| | | FSLKLSSVTAADTAVYYCARLGAFD | | RSNWHTFGQGTKLEIK |
| | | AFDIWGQGTMVTVSS | | |
| U.S. Pat. No. | 18 | QVQLVESGGGVVQPGGSLRLSCAAS | 19 | DIQMTQSPSSLSASVGDRVTITCRASQGISS |
| 9,228,016B2 | | GFILSDYGMHWVRQAPGKGLEWVT | | WLAWYQQKPEKAPKSLIYAASSLQSGVPS |
| | | VIWYDGSNKFYVDSVKGRFTISRDNS | | RFSGSGSGTDFTLTISSLQPEDFATYYCQQ |
| | | KNTLYLQMNSLRVEDTAVYYCARG | | YNSYPYTFGQGTKLEIK |
| | | GRLATGHFYYVMDVWGQGTTVTVS | | |
| | | S | | |
| U.S. Pat. No. | 20 | QVQLVESGGGVVQPGRSLRLSCTAS | 21 | AIQLTQSPSSLSASVGDRVTITCRASQGISS |
| 9,228,016B2 | | GFTFSSYGMQWVRQAPGKGLEWVA | | ALAWYQQKPGKAPKFLIYDASSLESGVPS |
| | | VIWYEGSNKYYADSVKGRFTISRENS | | RFSGSGSGTDFTLTISSLQPEDFATYYCQQ |
| | | KNTLYLQMNSLRAEDTAVYYCARG | | FNSYPYTFGQGTKLEIK |
| | | GLMVRGLFYYGMDVWGQGTTVTVS | | |
| | | S | | |
| U.S. Pat. No. | 22 | EVQLVESGGGLVKPGGSLRLSCAAS | 23 | EIVLTQSPGTLSLSPGERATLSCRASQSVSS |
| 9,228,016B2 | | GFTFSTVWMSWVRQAPGKGLEWVG | | SYLAWYQQKPGQAPRLLIYGASSRATGIP |
| | | RIKSKTDGGTTDYAAPVKGRFTISRD | | DRFSGSGSGTDFTLTISRLEPEDFAVYYCQ |
| | | DSKNTLYLQMNSLHTEDTAVYYCTT | | QYGSSPWTFGQGTKVEIK |
| | | GQLIPYSYYYGMDVWGQGTSVTVSS | | |
| U.S. Pat. No. | 22 | EVQLVESGGGLVKPGGSLRLSCAAS | 24 | EIVLTQSPGTLSLSPGERATLSCRASQSVTS |
| 9,228,016B2 | | GFTFSTVWMSWVRQAPGKGLEWVG | | SYLAWYQQKPGQAPRLLIYGASSRATGIP |
| | | RIKSKTDGGTTDYAAPVKGRFTISRD | | ERFSGSGSGTDFTLTISRLEPEDFAVYYCQ |
| | | DSKNTLYLQMNSLHTEDTAVYYCTT | | QYGSSPITFGQGTRLEIK |
| | | GQLIPYSYYYGMDVWGQGTSVTVSS | | |
| U.S. Pat. No. | 25 | QVQLQQWGAGLLKPSETLSLTCAVY | 26 | DIQMTQSPSSLSASVGDRVTITCRAS QGISS |
| 9,228,016B2 | | GGSFSGYYWSWIRQPPGKGLEWIGEI | | WLAWYQQKPEKAPKSLIYAASSLQSGVPS |
| | | NHSGNTYYNPSLKSRVTISVDTSKNQ | | RFSGSGSGTDFTLTISSLQPEDFATYYCQQ |
| | | LSLKLSSVTAADTAVYYCARFGSND | | YNSYPPTFGQGTKVEIK |
| | | AFDIWGQGTMVTVSS | | |
| U.S. Pat. No. | 27 | QVQLVESGGGVVQPGRSLRLSCAAS | 28 | AIQLTQSPSSLSASVGDRVTITCRASQGISS |
| 9,228,016B2 | | GFTFSNYGMHWVRQAPGKGLEWMA | | ALAWYQQKPGKAPKFLIYDASSLESGVPS |
| | | VIWYGGSNKFYADSVKGRFTISRDNS | | RFSGSGSGTDFTLTISSLQPEDFATYYCQQ |
| | | KNSLSLQMNSLRAEDTAVYYCARGG | | FNSYPQTFGQGTKVEIK |
| | | AMVRGVYYYGMDVWGQGTTVTVS | | |
| | | S | | |
| U.S. Pat. No. | 27 | QVQLVESGGGVVQPGRSLRLSCAAS | 29 | EIVLTQSPATLSLSPGERATLSCRASQSVSS |
| 9,228,016B2 | | GFTFSNYGMHWVRQAPGKGLEWMA | | YLAWYQQKPGQAPRLLIYDASNRATGIPA |
| | | VIWYGGSNKFYADSVKGRFTISRDNS | | RFSGSGSGTDFTLTISSLEPEDFAVYYCQQ |
| | | KNSLSLQMNSLRAEDTAVYYCARGG | | RSNWPLTFGGGTKVEIK |
| | | AMVRGVYYYGMDVWGQGTTVTVS | | |
| | | S | | |
| U.S. Pat. No. | 30 | QVQLVESGGDVVQPGRSLRLSCAAS | 31 | AIQLTQSPSSLSASVGDRVTITCRASQGISS |
| 9,228,016B2 | | GFTFSTYGMHWVRQAPGKGLEWVA | | ALAWYQQKPGKAPKLLIYDASSLESGVPS |
| | | VTWYAGSNKFYADSVKGRFTISRDN | | RFSGSGSGTDFTLTISSLQPEDFATYYCQQ |
| | | SKNTLYLQMNSLRAEDTAVYYCARG | | FNSYPYTFGQGTKLEIK |
| | | GSMVRGLYYYGMDVWGQGTTVTVS | | |
| | | S | | |
| U.S. Pat. No. | 32 | QVQLVQSGAEVKKPGASVKVSCKGS | 33 | DIVMTQSPPTLSLSPGERVTLSCKSSQSLLN |
| 20150368349A1 | | GYTFTDYAMYWVRQAPGQGLEWIG | | SGNQKNYLTWYQQKPGQAPRLLIYWAST |
| | | VIRTYSGDVTYNQKFKDRATMTVDK | | RESGIPARFSGSGSGTDFTLTISSLQPEDFA |
| | | SISTAYMELSRLRSDDTAVYYCAKSG | | VYHCQNDYSYPYTFGQGTKLEIK |
| | | TVRGFAYWGQGTLVTVSS | | |
| U.S. Pat. No. | 32 | QVQLVQSGAEVKKPGASVKVSCKGS | 34 | DIVMTQSPDSLAVSLGERATINCKSSQSLL |
| 20150368349A1 | | GYTFTDYAMYWVRQAPGQGLEWIG | | NSGNQKNYLTWYQQKPGQPPKLLIYWAS |
| | | VIRTYSGDVTYNQKFKDRATMTVDK | | TRESGVPDRFSGSGSGTDFTLTISSLQAED |
| | | SISTAYMELSRLRSDDTAVYYCAKSG | | VAVYHCQNDYSYPYTFGQGTKLEIK |
| | | TVRGFAYWGQGTLVTVSS | | |
| U.S. Pat. No. | 35 | QVQLVQSGAEVKKPGASVKVSCKAS | 36 | DIVMTQSPDSLAVSLGERATINCKSSQSLL |
| 20150368349A1 | | GYTFTGYAMYWVRQAPGQGMEWIG | | NSGNQKNYLSWYHQKPGQPPKMLIYWAS |
| | | VIRTFSGDVTYNQKFRGRATMTVDT | | TRESGVPDRFSGSGSGTDFTLTISSVQAED |
| | | SISTAYMELSRLRSDDTAVYYCAKSG | | VAVYHCQNDHSFPYTFGQGTKLEIK |
| | | TVRGFAYWGQGTLVTVSS | | |
| U.S. Pat. No. | 37 | QVQVVESGGGVVQPGRSLRLSCAAS | 38 | DIQMTQSPSSLSASVGDRVTITCRASQGIR |
| 20150064204 | | GFTFSSYGMHWVRQAPGKGLEWVS | | NDLGWYQQKPGKAPKRLIYDASSLQSGVP |
| | | VIWYEGSNKYYADSVKGRFTISRDNS | | SRFSGSGSGTEFTLTISSLQPEDFATYYCLQ |
| | | KNTLYLQMNSLRAEDTAVYYCARG | | HHSYPWTFGQGTKVEIKR |
| | | GLLGYYYYYGMDVWGQGTTVTVSS | | |
| U.S. Pat. No. | 39 | QVQLVESGGGVVQPGRSLRLSCAAS | 40 | DIQMTQSPSSLSASVGDRVTVTCRASQGIR |
| 20150064204 | | GFTFSSYGMHWVRQAPGKGLEWVA | | NDLGWYQQKPGKAPKRLIYAASSLQSGVP |
| | | VIWYPGSNKYYADSVKGRFTISRDNS | | SRFSGSGSGTEFTLTISSLQPEDFATYYCLQ |
| | | KNTLYLQMNSLRAEDTAVYYCARG | | HNNYPWTFGQGTKVDIKR |
| | | GELGRYYYYGMDVWGQGTTVTVSS | | |
| U.S. Pat. No. | 41 | QVQLQESGPGLVKPSQTLSLTCTVSG | 42 | EIVLTQSPGTLSLSPGERATLSCRASQTVSS |
| 20150064204 | | GSISSGGYFWSWIRQHPGKGLEWIGY | | NYLAWYQQKPGQAPRLLIYGSSTRATGIP |
| | | IYYSGTTYYNPSLKSRVTISIDTSKNH | | DRFSGSGSGTDFTLTISRLEPEDFAVYYCQ |
| | | FSLKLSSVTAADTAVYYCARDLFYY | | QYDSSPWTFGQGTKVEIKR |
| | | DSSGPRGFDPWGQGTLVTVSS | | |
| U.S. Pat. No. | 43 | QVQLVESGGGVVQPGRSLRLSCAAS | 44 | DIQMTQSPSSLSASVGDRVTITCRASQGIR |
| 20150064204 | | GFTFSSYGMHWVRQAPGKGLEWMA | | NDLGWYQQKPGKAPKRLIYAASSLQSGVP |
| | | VIWYVGSNKYYADSVKGRFTISRDN | | SRFSGSGSGTEFTLTISSLQPEDFATYYCQQ |
| | | SKNTLYLQMNSLSAEDTAVYYCARG | | HNSYPWTFGQGTKVEIKR |
| | | GELGRDYYSGMDVWGQGTTVTVSS | | |
| U.S. Pat. No. | 45 | QVQLVESGGGVVQPGRSLRLSCVAS | 46 | DIQMTQSPSSLSASVGDRVTITCRASQGIR |
| 20150064204 | | GFTFSSYGMHWIRQAPGKGLEWVAV | | NDLGWYQQKPGKAPNRLIYATSSLQSGVP |
| | | IWYEGSNKYYADSVKGRFTISRDNSK | | SRFSGSGSGTEFTLTISSLQPEDFATYYCLQ |
| | | NTLYLQMNSLRAEDTAVYYCARGG | | HNTYPWTFGQGTKVEIKR |
| | | RLGKDYYSGMDVWGQGTTVTVSS | | |
| U.S. Pat. No. | 47 | QVQLQESGPGLVKPSETLSLTCTVSG | 48 | EIVLTQSPGTLSLSPGERATLSCRASQTVSS |
| 20150064204 | | GSISSGGYFWSWIRQPPGKGLEWIGY | | NYLAWYQQKPGQAPRLLIYGSSTRATGIP |
| | | IYYSGTTYYNPSLKSRVTISIDTSKNQ | | DRFSGSGSGTDFTLTISRLEPEDFAVYYCQ |
| | | FSLKLSSVTAADTAVYYCARDLFYY | | QYDSSPWTFGQGTKVEIKR |
| | | DTSGPRGFDPWGQGTLVTVSS | | |
| U.S. Pat. No. | 49 | QVQLVESGGGVVQPGRSLRLSCAAS | 50 | DIQMTQSPSSLSASVGDRVTITCRASQGIR |
| 20150064204 | | GFTFSSYGMHWVRQAPGKGLEWMA | | NDLGWYQQKPGKAPKRLIYAASSLQSGVP |
| | | VIWYVGSNKYYADSVKGRFTISRDN | | SRFSGSGSGTEFTLTISSLQPEDFATYYCQQ |
| | | SKNTLYLQMNSLRAEDTAVYYCARG | | HNSYPWTFGQGTKVEIKR |
| | | GELGRDYYSGMDVWGQGTTVTVSS | | |
| U.S. Pat. No. | 51 | QVQLVESGGGVVQPGRSLRLSCVAS | 52 | DIQMTQSPSSLSASVGDRVTITCRASQGIR |
| 20150064204 | | GFTFSSYGMHWIRQAPGKGLEWVAV | | NDLGWYQQKPGKAPKRLIYATSSLQSGVP |
| | | IWYEGSNKYYAESVKGRFTISRDNSK | | SRFSGSGSGTEFTLTISSLQPEDFATYYCLQ |
| | | NTLYLQMNSLRAEDTAVYYCARGG | | HNTYPWTFGQGTKVEIKR |
| | | RLGKDYYSGMDVWGQGTTVTVSS | | |
| U.S. Pat. No. | 53 | QVQVVESGGGVVQPGRSLRLSCAAS | 54 | DIQMTQSPSSLSASVGDRVTITCRASQGIR |
| 20150064204 | | GFTFSSYGMHWVRQAPGKGLEWVS | | NDLGWYQQKPGKAPKRLIYDASSLQSGVP |
| | | VIWYEGSNKYYAESVKGRFTISRDNS | | SRFSGSGSGTEFTLTISSLQPEDFATYYCLQ |
| | | KNTLYLQMNSLRAEDTAVYYCARG | | HHSYPWTFGQGTKVEIKR |
| | | GLLGYYYYYGMDVWGQGTTVTVSS | | |
| U.S. Pat. No. | 55 | QVQLVESGGGVVQPGRSLRLSCAAS | 56 | DIQMTQSPSSLSASVGDRVTVTCRASQGIR |
| 20150064204 | | GFTFSSYGMHWVRQAPGKGLEWVA | | NDLGWYQQKPGKAPKRLIYAASSLQSGVP |
| | | VIWYPGSNKYYAESVKGRFTISRDNS | | SRFSGSGSGTEFTLTISSLQPEDFATYYCLQ |
| | | KNTLYLQMNSLRAEDTAVYYCARG | | HNNYPWTFGQGTKVDIKR |
| | | GELGRYYYYGMDVWGQGTTVTVSS | | |
| U.S. Pat. No. | 57 | EVQLVQSGGGLVQPGGSLRLSCSAS | 58 | EIVMTQSPSSLSASVGDRVTITCRASQSINN |
| 20140072565A1 | | GFSFSSYAMHWVRQAPGKGLEYVSG | | YLNVVYQQKPGKAPKLLIYATSRLQSGVPS |
| | | ISDNGGSTKYADSVKGRFTISRDNSQ | | RFSGSGSGADLTLTISSLQPEDVATYYCQQ |
| | | NTLYLQMSSLRSEDTAVYYCARGGP | | SYSFPWTFGQGTKVEIK |
| | | TYYDFWSGYYTDEDAFDIWGQGTLV | | |
| | | TVSS | | |
| U.S. Pat. No. | 59 | EVQLEVQLVQSGGGLVQPGGSLRLS | 60 | EIVMTQSPSSLSASVGDRVTITCRASQSINN |
| 20140072565A1 | | CSASGFSFSSYAMHWVRQAPGKGLE | | YLNVVYQQKPGKAPKLLIYATSRLQSGVPS |
| | | YVSGISDNGGSTKYADSVKGRFTISR | | RFSGSGSGADLTLTISSLQPEDVATYYCQQ |
| | | DNSQNTLYLQMSSLRSEDTAVYYCA | | SYSFPWTFGQGTKVEIK |
| | | RGGPTYYDFWSGYYTDEDAFDIWGQ | | |
| | | GTLVTVSS | | |
| U.S. Pat. No. | 61 | QVQLVQSGTQVKMPGASVKVSCKA | 62 | QSVVTQPPSVSAAPGQKVTISCSGSTSNIG |
| 20140072565A1 | | SGYTFDDYGIGWVRQAPGQGLEWM | | NNYVSWYQQLPGTAPKLLIYDNYKRPSGI |
| | | GWISPYTHRTNSSPKLQDRVTMTTDT | | PDRFSGSKSGTSATLGITGLRTGDEADYFC |
| | | STSTAYMELRSLRSDDTAVYYCARD | | GTWDSSLNAWVFGGGTKLTVL |
| | | GTYYDFWSGYFDNGAFDIWGQGTL | | |
| | | VTVSS | | |
| U.S. Pat. No. | 63 | EVQLLESGGGLIQPGGSLRLSCAASG | 64 | SYELMQPPSVSVSPGQTAGITCSGDALPKQ |
| 20140072565A1 | | FTFSTYGMSWVRQAPGKGLEWVSGI | | YAYWYQQRPGQAPVLLIYKDTERPSGIPE |
| | | TGSAGGGSTNYADSVKGRFTISRDNS | | RFSGSSSGTTVTLTISGVQAEDEADYYCQS |
| | | KNTLYLQMNSLRAEDTAVYYCAKG | | ADSSGTYPVFGGGTKLTVL |
| | | YSSNWRSAFDIWGQGTMVTVSS | | |
| U.S. Pat. No. | 65 | QVQLVESGGGVVQPGRSLRLSCAAS | 66 | DIQMTQSPSSLSASVGDRVTITCRASQTIY |
| 20140072565A1 | | GFTFSSYAMHWVRQAPGKGLEWVA | | NYLNWYQQKPGKAPKLLIYAASSLQSGVP |
| | | VISYDGSNKYYADSVKGRFTISRDNS | | SRFGGRGYGTDFTLTINSLQPEDFATYFCQ |
| | | KNTLYLQMNSLRAEDTAVYYCARGI | | QSYTSPLTFGQGTKVDIK |
| | | AAAGPPYYYYYYYMDVWGKGTTVT | | |
| | | VSS | | |
| U.S. Pat. No. | 67 | QVTLVESGGGLVKPGGSLTLSCGAS | 68 | DIVLTQSPASLAASVGDRATISCRASETVD |
| 20130108641A1 | | GFTISSYAMSWVRQSPGKALEWVAII | | NYGISFMNVVFQQKPGKSPKLLIYAASNQG |
| | | STGGSTYYPDSVRGRFTISRDNAKNS | | SGVPARFSGSGSGTDFSLNIHPMQPDDTAT |
| | | LYLTMSSLDSVDTAMYYCARVGGY | | YFCQQSKEVPWTFGGGTKLE |
| | | YDSMDHWGQGTSVT | | |
| U.S. Pat. No. | 69 | QVTLVESGGGLVKPGGSLTLSCGAS | 68 | DIVLTQSPASLAASVGDRATISCRASETVD |
| 20130108641A1 | | GFTISSYAMSWVRQSPGKALEWVAII | | NYGISFMNVVFQQKPGKSPKLLIYAASNQG |
| | | STGGSTYYPDSVRGRFTISRDNAKNS | | SGVPARFSGSGSGTDFSLNIHPMQPDDTAT |
| | | LYLTMSSLDSVDTATYYCARVGGYY | | YFCQQSKEVPWTFGGGTKLE |
| | | DSMDHWGQGTSVT | | |
| U.S. Pat. No. | 70 | QVTLVESGGGLVKPGGSLTLSCGAS | 71 | DIVLTQSPASLSASVGDRATISCRASETVD |
| 20130108641A1 | | GFTISSYAMSWVRQSPGKALEWVAII | | NYGISFMNVVFQQKPGQSPKLLIYAASNQG |
| | | STGGSTYYPDKFRGRFTISRDNAKNS | | SGVPARFSGSGSGTDFSLTISPMQPDDTAT |
| | | LYLTMSSLRSEDTATYYCARVGGYY | | YYCQQSKEVPWTFGGGTKLE |
| | | DSMDHWGQGTSVT | | |
| U.S. Pat. No. | 72 | QVTLKESGGGLVKPGGSLTLSCGAS | 71 | DIVLTQSPASLSASVGDRATISCRASETVD |
| 20130108641A1 | | GFTISSYAMSWVRQSPGKALEWVAII | | NYGISFMNVVFQQKPGQSPKLLIYAASNQG |
| | | STGGSTYYPDKFRGRFTISRDNAKNS | | SGVPARFSGSGSGTDFSLTISPMQPDDTAT |
| | | LYLTMSSLRSEDTATYYCARVGGYY | | YYCQQSKEVPWTFGGGTKLE |
| | | DSMDHWGQGTSVT | | |
| U.S. Pat. No. | 73 | EVQLVESGGGLIQPGGSLKLSCAASG | 74 | DIVLTQSPASLAVSPGQRATITCRASETVD |
| 20130108641A1 | | FTISSYAMSWVRQAPGKGLEWVAIIS | | NYGISFMNVVFQQKPGQPPKLLIYAASNQG |
| | | TGGSTYYADSVKGRFTISRDNSKNTL | | SGVPARFSGSGSGTDFTLTINPVEADDTAN |
| | | YLQMNSLRAEDTAVYYCARVGGYY | | YYCQQSKEVPWTFGQGTKVE |
| | | DSMDHWGQGTSVT | | |
| U.S. Pat. No. | 75 | QVQLVESGGGVVQPGRSLRLSCAXS | 76 | DIVMTQSPLSLPVTPGEPASISCRSSQSLVH |
| 8,709,424B2 | | GFSLSTSGMGVGWVRQAPGKGLEW | | SDGNTYLHWYLQKPGQSPQLLIYKVSKRF |
| | | VAHIWWDDDKYYSPSLKSRXTISXD | | SGVPDRFSGSGSGTDFTLKISRVEAEDVGV |
| | | XSKNTXYLQMNSLRAEDTAVYYCX | | YYCSQSTHVPPTFGQGTKVEIKR |
| | | RSYYYGSSGAMDYWGQGTLVTVSS | | |
| U.S. Pat. No. | 77 | QVQLVESGGGVVQPGRSLRLSCAAS | 78 | DIVMTQSPLSLPVTPGEPASISCRSSQSLLH |
| 8,709,424B2 | | GFTFSDYYMAWVRQAPGKGLEWVA | | SDGNTFLSWYLQKPGQSPQLLIYLASNRFS |
| | | YIHANGGSTYYRDSVRGRFTISRDNS | | GVPDRFSGSGSGTDFTLKISRVEAEDVGV |
| | | KNTLYLQMNSLRAEDTAVYYMCG | | YYCFQHTHLPLTFGQGTKVEIKR |
| | | SFMYAADYYIMDAWGQGTLVTVSS | | |
| U.S. Pat. No. | 79 | QVQLVQSGAEVKKPGASVKVSCKAS | 80 | EIVLTQSPGTLSLSPGERATLSCTASSSVSS |
| 8,709,424B2 | | GYTFSRYWIEWVRQAPGQGLEWXG | | SYFHWYQQKPGQAPRLXIYSTSNLASGIPD |
| | | EILPGSGSSNYNEKFKDRXTXTXDTS | | RFSGSGSGTDXTLTISRLEPEDFAVYYCHQ |
| | | TSTAYMELRSLRSDDTAVYYCARKV | | YHRSPRTFGQGTKVEIKR |
| | | YYYAMDFWGQGTLVTVSS | | |
| U.S. Pat. No. | 81 | QVQLQESGPGLVKPSETLSLTCTXSG | 82 | DIQMTQSPSSLSASVGDRVTITCRASQGVN |
| 8,709,424B2 | | FSLSTYGVGVGWIRQPPGKGLEWXX | | NFLTWYQQKPGKAPKXLIXYTSNLQSGVP |
| | | NIWWDDDNYYNPSLIHRXTXSXDTS | | SRFSGSGSGTDXTLTISSLQPEDFATYYCQ |
| | | KNQXSLKLSSVTAADTAVYYCAXIK | | QYHGFPNTFGQGTKVEIKR |
| | | EPRDWFFEFWGQGTLVTVSS | | |
| U.S. Pat. No. | 83 | QVQLQESGPGLVKPSETLSLTCTXSG | 84 | DIQMTQSPSSLSASVGDRVTITCRASQGVN |
| 8,709,424B2 | | FSLSTYGVGVGWIRQPPGKGLEWXX | | NYLTWYQQKPGKAPKXLIXYTSNLQSGVP |
| | | NIWWDDDKYYNPSLKNRXTISXDTS | | SRFSGSGSGTDXTLTISSLQPEDFATYYCQ |
| | | KNQXSLKLSSVTAADTAVYYCAXIK | | QYHGFPNTFGQGTKVEIKR |
| | | EPRDWFFEFWGQGTLVTVSS | | |
| U.S. Pat. No. | 85 | QVQLVESGGGVVQPGRSLRLSCAAS | 86 | DIQMTQSPSSLSASVGDRVTITCRASQDIN |
| 8,709,424B2 | | GFTVRNYAMSWVRQAPGKGLEWVA | | NFLNWYQQKPGKAPKLLIYYTSKLHSGVP |
| | | SISTGDRSYLPDSMKGRFTISRDNSKN | | SRFSGSGSGTDFTLTISSLQPEDFATYYCQ |
| | | TLYLQMNSLRAEDTAVYYCXRYFDF | | QGHTLPPTFGQGTKVEIKR |
| | | DSFAFWGQGTLVTVSS | | |
| U.S. Pat. No. | 87 | QVQLQESGPGLVKPSETLSLTCTVSG | 88 | DIVMTQSPDSLAVSLGERATINCKASQDV |
| 8,709,424B2 | | DSITSGYWNWIRQPPGKGLEXXGYIS | | NTAVAWYQQKPGQPPKLLIYWASTRHTG |
| | | YSGSTYYNPSLRGRVTISXDTSKNQF | | VPDRFSGSGSGTDXTLTISSLQAEDVAVYY |
| | | SLKLSSVTAADTAVYYCXRRHLGSG | | CQQHSYTPPWTFGQGTKVEIKR |
| | | YGWFAYWGQGTLVTVSS | | |
| U.S. Pat. No. | 89 | QVQLVESGGGVVQPGRSLRLSCAAS | 90 | EIVLTQSPGTLSLSPGERATLSCRASESVDX |
| 8,709,424B2 | | GFTFSSYAMSWVRQAPGKGLEWVA | | YGVSFMNWYQQKPGQAPRLLIYAASXQG |
| | | SISSGGTTYYPDSVKGRFTISRDNSKN | | SGIPDRFSGSGSGTDFTLTISRLEPEDFAVY |
| | | TLYLQMNSLRAEDTAVYYCARVGG | | YCQQTKEVTWTFGQGTKVEIKR |
| | | YYDSMDYWGQGTLVTVSS | | |
| U.S. Pat. No. | 91 | QVQLQESGPGLVKPSETLSLTCTVSG | 92 | XIVMTQSPDSLAVSLGERATINCKASQDVI |
| 8,709,424B2 | | DSITSGYWNWIRQPPGKGLEXXGFIS | | SAVAWYQQKPGQPPKLLIYWASTRHTGV |
| | | YSGNTYYNPSLRSRXTISXDTSKNQX | | PDRFSGSGSGTDFTLTISSLQAEDVAVYYC |
| | | SLKLSSVTAADTAVYYCXRRHLISGY | | QQHSYTPPWTFGQGTKVEIKR |
| | | GWFAYWGQGTLVTVSS | | |
| U.S. Pat. No. | 93 | QVQLVQSGAEVKKPGASVKVSCKAS | 94 | DIQMTQSPSSLSASVGDRVTITCRASESVD |
| 8,709,424B2 | | GYTFTSYTMHWVRQAPGQGLEWXG | | NYGISFMNWYQQKPGKAPKLLIYAASNQ |
| | | YINPRSVYTNYNQKFKDRXTXTXDX | | GSGVPSRFSGSGSGTDFTLTISSLQPEDFAT |
| | | STSTAYMELRSLRSDDTAVYYCARL | | YYCQQSKEVPFTFGQGTKVEIKR |
| | | GGYYDTMDYWGQGTLVTVSS | | |
| U.S. Pat. No. | 95 | QVQLQESGPGLVKPSETLSLTCTVSG | 96 | EIVLTQSPGTLSLSPGERATLSCSANSTVN |
| 8,709,424B2 | | YSITSDYAWNWIRQPPGKGLEWXGY | | YMYWYQQKPGQAPR)OUYLTSNLASGIPD |
| | | ISYSGSTRYNPSLKSRXTISXDTSKNQ | | RFSGSGSGTDFTLTISRLEPEDFAVYYCQQ |
| | | FSLKLSSVTAADTAVYYCARQLGLR | | WNSNPPTFGQGTKVEIKR |
| | | FFDYWGQGTLVTVSS | | |
| U.S. Pat. No. | 97 | QVTLRESGPALVKPTQTLTLTCTFSG | 98 | EIVMTQSPATLSVSPGERATLSCKASQNVG |
| 7,812,135B2 | | FSLSTSGMGVGWIRQPPGKALEWLA | | TNVAWYQQKPGQAPRLLIYSASYRYSGIP |
| | | HIWWDDDKYYNPSLKSRLTISKDTS | | ARFSGSGSGTEFTLTISSLQSEDFAVYYCQ |
| | | KNQVVLTMTNMDPVDTATYYCART | | QYNTDPLTFGGGTKVEIK |
| | | RRYFPFAYWGQGTLVTVSS | | |
| U.S. Pat. No. | 99 | QVTLRESGPALVKPTQTLTLTCTFSG | 98 | EIVMTQSPATLSVSPGERATLSCKASQNVG |
| 7,812,135B2 | | FSLSTSGMGVGWIRQPPGKALEWLA | | TNVAWYQQKPGQAPRLLIYSASYRYSGIP |
| | | HIWWDDDKYYQPSLKSRLTISKDTS | | ARFSGSGSGTEFTLTISSLQSEDFAVYYCQ |
| | | KNQVVLTMTNMDPVDTATYYCART | | QYNTDPLTFGGGTKVEIK |
| | | RRYFPFAYWGQGTLVTVSS | | |
| W02016057841 | 100 | QVQLVESGGGLVQPGGSLRLSCAAS | 101 | EIVMTQSPATLSVSPGERATLSCRASESVS |
| | | GFSLSSYGVDWVRQAPGKGLEWVG | | SNVAWYQQRPGQAPRLLIYGASNRATGIP |
| | | VIWGGGGTYYASSVMARFTISRDNS | | ARFSGSGSGTDFTLTISRLEPEDFAVYYCG |
| | | KNTLYLQMNSLRAEDTAVYYCAKH | | QSYSYPFTFGQGTKLEIK |
| | | AYGHDGGFAMDYWGQGTLVTVSS | | |
| W02016057841 | 102 | QVQLVESGGGLVQPGGSLRLSCAAS | 103 | EIVMTQSPATLSVSPGERATLSCRASQSVS |
| | | GFSLSSYGVDWVRQAPGKGLEWLG | | SNLAWYQQKPGQAPRLLIYGASNRATGIP |
| | | VIWGGGGTYYTASLMGRFTISRDNS | | DRFSGSGSGTDFTLTISRLEPEDFAVYYCG |
| | | KNTLYLQMNSLRAEDTAVYYCAKH | | QSYSYPFTFGQGTKLEIK |
| | | AYGHDGGFAMDYWGQGTLVTVSS | | |
| W02016057841 | 104 | EVQLVESGGGLVQSGGSLRLSCAAS | 101 | EIVMTQSPATLSVSPGERATLSCRASESVS |
| | | GFSLSSYGVDWVRQAPGKGLEWVG | | SNVAWYQQRPGQAPRLLIYGASNRATGIP |
| | | VIWGGGGTYYASSLMGRFTISRDNS | | ARFSGSGSGTDFTLTISRLEPEDFAVYYCG |
| | | KNTLYLQMNSLRAEDTAVYYCAKH | | QSYSYPFTFGQGTKLEIK |
| | | AYGHDGGFAMDYWGQGTLVTVSS | | |
| W02016057841 | 105 | EVQLVESGGGLVQSGGSLRLSCAAS | 101 | EIVMTQSPATLSVSPGERATLSCRASESVS |
| | | GFSLSSYGVDWVRQAPGKGLEWLG | | SNVAWYQQRPGQAPRLLIYGASNRATGIP |
| | | VIWGGGGTYYTSSLMGRFTISRDNSK | | ARFSGSGSGTDFTLTISRLEPEDFAVYYCG |
| | | NTLYLQMNSLRAEDTAVYYCAKHA | | QSYSYPFTFGQGTKLEIK |
| | | YGHDGGFAMDYWGQGTLVTVSS | | |
| W02016057841 | 106 | EVQLVESGGGLVQSGGSLRLSCAAS | 101 | EIVMTQSPATLSVSPGERATLSCRASESVS |
| | | GFSLSSYGVDWVRQAPGKGLEWLG | | SNVAWYQQRPGQAPRLLIYGASNRATGIP |
| | | VIWGGGGTYYTSSLMARFTISRDNSK | | ARFSGSGSGTDFTLTISRLEPEDFAVYYCG |
| | | NTLYLQMNSLRAEDTAVYYCAKHA | | QSYSYPFTFGQGTKLEIK |
| | | YGHDGGFAMDYWGQGTLVTVSS | | |
| W02016057841 | 107 | EVQLVESGGGLVQSGGSLRLSCAAS | 101 | EIVMTQSPATLSVSPGERATLSCRASESVS |
| | | GFSLSSYGVDWVRQAPGKGLEWVG | | SNVAWYQQRPGQAPRLLIYGASNRATGIP |
| | | VIWGGGGTYYASSLMGRFTISRDNS | | ARFSGSGSGTDFTLTISRLEPEDFAVYYCG |
| | | KNTLYLQMNSLRAEDTAVYYCARH | | QSYSYPFTFGQGTKLEIK |
| | | AYGHDGGFAMDYWGQGTLVTVSS | | |
| W02016057841 | 108 | EVQLVESGGGLVQSGGSLRLSCAAS | 101 | EIVMTQSPATLSVSPGERATLSCRASESVS |
| | | GFSLSSYGVDWVRQAPGKGLEWVG | | SNVAWYQQRPGQAPRLLIYGASNRATGIP |
| | | VIWGGGGTYYASSLMGRFTISRDNS | | ARFSGSGSGTDFTLTISRLEPEDFAVYYCG |
| | | KNTLYLQMNSLRAEDTAVYYCARN | | QSYSYPFTFGQGTKLEIK |
| | | AYGHDGGFAMDYWGQGTLVTVSS | | |

In certain aspects the VH can comprise an amino acid sequence at least 60%, at least 65%, at least 70%, at least 75%, at least 80%, at least 85%, at least 90%, at least 95% or 100% identical to the mature VH amino acid sequence comprising or contained within SEQ ID NO: 9, SEQ ID NO: 11, SEQ ID NO: 13, SEQ ID NO: 15, SEQ ID NO: 18, SEQ ID NO: 20, SEQ ID NO: 22, SEQ ID NO: 25, SEQ ID NO: 27, SEQ ID NO: 30, SEQ ID NO: 32, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 70, SEQ ID NO: 72, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 100, SEQ ID NO: 102, SEQ ID NO: 104, SEQ ID NO: 105, SEQ ID NO: 106, SEQ ID NO: 107, SEQ ID NO: 108, or SEQ ID NO: 109.

In certain aspects the VL can comprise an amino acid sequence at least at least 60%, at least 65%, at least 70%, at least 75%, at least 80%, at least 85%, at least 90%, at least 95% or 100% identical to the mature VL amino acid sequence comprising or contained within SEQ ID NO: 10, SEQ ID NO: 12, SEQ ID NO: 14, SEQ ID NO: 16, SEQ ID NO: 17, SEQ ID NO: 19, SEQ ID NO: 21, SEQ ID NO: 23, SEQ ID NO: 24, SEQ ID NO: 26, SEQ ID NO: 28, SEQ ID NO: 29, SEQ ID NO: 31, SEQ ID NO: 33, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 71, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 101, SEQ ID NO: 103, or SEQ ID NO: 110.

In certain aspects the VH and VL amino acid sequences can comprise amino acid sequences at least at least 60%, at least 65%, at least 70%, at least 75%, at least 80%, at least 85%, at least 90%, at least 95% or 100% identical to the mature VH and VL amino acid sequences comprising or contained within SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 15 and SEQ ID NO: 17; SEQ ID NO: 18 and SEQ ID NO: 19; SEQ ID NO: 20 and SEQ ID NO: 21; SEQ ID NO: 22 and SEQ ID NO: 23; SEQ ID NO: 22 and SEQ ID NO: 24; SEQ ID NO: 25 and SEQ ID NO: 26; SEQ ID NO: 27 and SEQ ID NO: 28; SEQ ID NO: 27 and SEQ ID NO: 29; SEQ ID NO: 30 and SEQ ID NO: 31; SEQ ID NO: 32 and SEQ ID NO: 33; SEQ ID NO: 32 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 57 and SEQ ID NO: 58; SEQ ID NO: 59 and SEQ ID NO: 60; SEQ ID NO: 61 and SEQ ID NO: 62; SEQ ID NO: 63 and SEQ ID NO: 64; SEQ ID NO: 65 and SEQ ID NO: 66; SEQ ID NO: 67 and SEQ ID NO: 68; SEQ ID NO: 69 and SEQ ID NO: 68; SEQ ID NO: 70 and SEQ ID NO: 71; SEQ ID NO: 72 and SEQ ID NO: 71; SEQ ID NO: 73 and SEQ ID NO: 74; SEQ ID NO: 75 and SEQ ID NO: 76; SEQ ID NO: 77 and SEQ ID NO: 78; SEQ ID NO: 79 and SEQ ID NO: 80; SEQ ID NO: 81 and SEQ ID NO: 82; SEQ ID NO: 83 and SEQ ID NO: 84; SEQ ID NO: 85 and SEQ ID NO: 86; SEQ ID NO: 87 and SEQ ID NO: 88; SEQ ID NO: 89 and SEQ ID NO: 90; SEQ ID NO: 91 and SEQ ID NO: 92; SEQ ID NO: 93 and SEQ ID NO: 94; SEQ ID NO: 95 and SEQ ID NO: 96; SEQ ID NO: 97 and SEQ ID NO: 98; SEQ ID NO: 99 and SEQ ID NO: 98; SEQ ID NO: 100 and SEQ ID NO: 101; SEQ ID NO: 102 and SEQ ID NO: 103; SEQ ID NO: 104 and SEQ ID NO: 101; SEQ ID NO: 105 and SEQ ID NO: 101; SEQ ID NO: 106 and SEQ ID NO: 101; SEQ ID NO: 107 and SEQ ID NO: 101; SEQ ID NO: 108 and SEQ ID NO: 101; or SEQ ID NO: 109 and SEQ ID NO: 110, respectively.

In certain aspects the GITR antigen binding domain of a dimeric, hexameric, or pentameric binding molecule as provided herein comprises the HCDR1, HCDR2, and HCDR3 regions, or HCDR1, HCDR2, and HCDR3 regions containing one or two single amino acid substitutions, and the LCDR1, LCDR2, and LCDR3 regions, or LCDR1, LCDR2, and LCDR3 containing one or two single amino acid substitutions, of the mature VH and VL amino acid sequences comprising or contained within SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 15 and SEQ ID NO: 17; SEQ ID NO: 18 and SEQ ID NO: 19; SEQ ID NO: 20 and SEQ ID NO: 21; SEQ ID NO: 22 and SEQ ID NO: 23; SEQ ID NO: 22 and SEQ ID NO: 24; SEQ ID NO: 25 and SEQ ID NO: 26; SEQ ID NO: 27 and SEQ ID NO: 28; SEQ ID NO: 27 and SEQ ID NO: 29; SEQ ID NO: 30 and SEQ ID NO: 31; SEQ ID NO: 32 and SEQ ID NO: 33; SEQ ID NO: 32 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 57 and SEQ ID NO: 58; SEQ ID NO: 59 and SEQ ID NO: 60; SEQ ID NO: 61 and SEQ ID NO: 62; SEQ ID NO: 63 and SEQ ID NO: 64; SEQ ID NO: 65 and SEQ ID NO: 66; SEQ ID NO: 67 and SEQ ID NO: 68; SEQ ID NO: 69 and SEQ ID NO: 68; SEQ ID NO: 70 and SEQ ID NO: 71; SEQ ID NO: 72 and SEQ ID NO: 71; SEQ ID NO: 73 and SEQ ID NO: 74; SEQ ID NO: 75 and SEQ ID NO: 76; SEQ ID NO: 77 and SEQ ID NO: 78; SEQ ID NO: 79 and SEQ ID NO: 80; SEQ ID NO: 81 and SEQ ID NO: 82; SEQ ID NO: 83 and SEQ ID NO: 84; SEQ ID NO: 85 and SEQ ID NO: 86; SEQ ID NO: 87 and SEQ ID NO: 88; SEQ ID NO: 89 and SEQ ID NO: 90; SEQ ID NO: 91 and SEQ ID NO: 92; SEQ ID NO: 93 and SEQ ID NO: 94; SEQ ID NO: 95 and SEQ ID NO: 96; SEQ ID NO: 97 and SEQ ID NO: 98; SEQ ID NO: 99 and SEQ ID NO: 98; SEQ ID NO: 100 and SEQ ID NO: 101; SEQ ID NO: 102 and SEQ ID NO: 103; SEQ ID NO: 104 and SEQ ID NO: 101; SEQ ID NO: 105 and SEQ ID NO: 101; SEQ ID NO: 106 and SEQ ID NO: 101; SEQ ID NO: 107 and SEQ ID NO: 101; SEQ ID NO: 108 and SEQ ID NO: 101; or SEQ ID NO: 109 and SEQ ID NO: 110, respectively.

In certain aspects the GITR antigen binding domain of a dimeric, hexameric, or pentameric binding molecule as provided herein comprises a VH comprising the amino acid sequence SEQ ID NO: 49 and a VL comprising the amino acid sequence SEQ ID NO: 50 (“anti-GITR #1”).

In certain aspects the GITR antigen binding domain of a dimeric, hexameric, or pentameric binding molecule as provided herein comprises a VH comprising the amino acid sequence SEQ ID NO: 9 and a VL comprising the amino acid sequence SEQ ID NO: 10 (“anti-GITR #2”).

By “mature VH amino acid sequence” or “mature VL amino acid sequence” is meant the VH or VL amino acid sequence remaining after the secretory signal peptide is cleaved off.

While a variety of different dimeric, pentameric, and hexameric binding molecules can be contemplated by a person of ordinary skill in the art based on this disclosure, and as such are included in this disclosure, in certain aspects, a binding molecule as described above is provided in which each binding unit comprises two IgA or IgM heavy chains each comprising a VH situated amino terminal to the IgA or IgM constant region or fragment thereof, and two immunoglobulin light chains each comprising a VL situated amino terminal to an immunoglobulin light chain constant region.

Moreover in certain aspects, at least one binding unit of the binding molecule, or at least two, at least three, at least four, at least five, or at least six binding units of the binding molecule, comprises or comprise two of the GITR binding domains as described above. In certain aspects the two GITR binding domains in the at least one binding unit of the binding molecule, or at least two, at least three, at least four, at least five, or at least six binding units of the binding molecule, can be different from each other, or they can be identical.

In certain aspects, the two IgA or IgM heavy chains within the at least one binding unit of the binding molecule, or at least two, at least three, at least four, at least five, or at least six binding units of the binding molecule, are identical. In certain aspects, two identical IgA or IgM heavy chains within at least one binding unit, or within at least two, at least three, at least four, at least five, or at least six binding units of the binding molecule comprise the heavy chain variable domain amino acid sequences as disclosed in Table 2.

In certain aspects, the two light chains within the at least one binding unit of the binding molecule, or at least two, at least three, at least four, at least five, or at least six binding units of the binding molecule, are identical. In certain aspects, two identical light chains within at least one binding unit, or within at least two, at least three, at least four, at least five, or at least six binding units of the binding molecule are kappa light chains, e.g., human kappa light chains, or lambda light chains, e.g., human lambda light chains. In certain aspects, two identical light chains within at least one binding unit, or within at least two, at least three, at least four, at least five, or at least six binding units of the binding molecule each comprise the light chain variable domain amino acid sequences as disclosed in Table 2.

In certain aspects at least one, at least two, at least three, at least four, at least five, or at least six binding units of a dimeric, pentameric, or hexameric binding molecule provided by this disclosure comprises or each comprise two identical IgA or IgM heavy chain constant regions each comprising identical heavy chain variable domain amino acid sequences as disclosed in Table 2, and two identical light chains each comprising identical heavy chain variable domain amino acid sequences as disclosed in Table 2. According to this aspect, the GITR binding domains in the at least one binding unit of the binding molecule, or at least two, at least three, at least four, at least five, or at least six binding units of the binding molecule, can be identical. Further according to this aspect, a dimeric, pentameric, or hexameric binding molecule as provided herein can comprise at least one, at least two, at least three, at least four, at least five, at least six, at least seven, at least eight, at least nine, at least ten, at least eleven, or at least twelve copies of a GITR binding domain as described above. In certain aspects at least two, at least three, at least four, at least five, or at least six of the binding units can be identical and, in certain aspects the binding units can comprise identical binding domains, e.g., at least two, at least three, at least four, at least five, at least six, at least seven, at least eight, at least nine, at least ten, at least eleven, or at least twelve GITR binding domains can be identical.

In certain aspects, a dimeric, pentameric, or hexameric GITR agonist binding molecule as provided herein can possess advantageous structural or functional properties compared to a corresponding bivalent binding molecule having the same antigen binding domains. In certain aspects a dimeric, pentameric, or hexameric binding molecule as provided herein can trigger activation of GITR-expressing cells, e.g., T cells, e.g., Tregs or activated effector CTLs, at higher potency than an equivalent amount of a monospecific, bivalent IgG antibody or fragment thereof comprising the same binding domains. In certain aspects a dimeric, pentameric, or hexameric binding molecule as provided herein can more efficiently cross-link multiple, e.g., three or more GITR receptors on the surface of a cell, and/or can effectively cross-link multiple, e.g., three or more GITR receptors on the surface of a cell in the absence of a secondary cross-linking moiety such as, but not limited to a FcγR, thereby facilitating anti-tumor immunity. Upon activation of the receptors by the binding of a dimeric, pentameric, or hexameric binding molecule as provided herein, the cell, e.g., a T cell, e.g., a Treg or an activated effector CTL, can be more effectively activated and in turn can induce improved anti-tumor immunity than an equivalent amount of a monospecific, bivalent IgG antibody or fragment thereof comprising the same binding domains, where the antibody comprises the same VH and VL regions as the antibodies provided in Table 2, or the antibody is e.g., TRX518, MK-4166, or INCAGN1876.

Polynucleotides, Vectors, and Host Cells

The disclosure further provides a polynucleotide, e.g., an isolated, recombinant, and/or non-naturally-occurring polynucleotide, comprising a nucleic acid sequence that encodes a polypeptide subunit of the dimeric, hexameric, or pentameric binding molecule as described above. By “polypeptide subunit” is meant a portion of a binding molecule, binding unit, or antigen binding domain that can be independently translated. Examples include, without limitation, an antibody variable domain, e.g., a VH or a VL, a J chain, a secretory component, a single chain Fv, an antibody heavy chain, an antibody light chain, an antibody heavy chain constant region, an antibody light chain constant region, and/or any fragment, variant, or derivative thereof.

In certain aspects, the polypeptide subunit can comprise an IgM or an IgA heavy chain constant region or fragment thereof, and VH portion of a GITR antigen binding domain. In certain aspects the polynucleotide can encode a polypeptide subunit comprising a human IgM or IgA constant region or fragment thereof fused to the C-terminal end of a VH, where the VH comprises the HCDR1, HCDR2, and HCDR3 regions, or the HCDR1, HCDR2, and HCDR3 regions containing one or two single amino acid substitutions of a VH comprising or contained within the amino acid sequence SEQ ID NO: 9, SEQ ID NO: 11, SEQ ID NO: 13, SEQ ID NO: 15, SEQ ID NO: 18, SEQ ID NO: 20, SEQ ID NO: 22, SEQ ID NO: 25, SEQ ID NO: 27, SEQ ID NO: 30, SEQ ID NO: 32, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 70, SEQ ID NO: 72, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 100, SEQ ID NO: 102, SEQ ID NO: 104, SEQ ID NO: 105, SEQ ID NO: 106, SEQ ID NO: 107, SEQ ID NO: 108, or SEQ ID NO: 109.

In certain aspects, the polypeptide subunit can comprise an antibody VL portion of a GITR antigen binding domain as described above. In certain aspects the polypeptide subunit can comprise a human antibody light chain constant region or fragment thereof fused to the C-terminal end of a VL, where the VL comprises LCDR1, LCDR2, and LCDR3 regions, or the LCDR1, LCDR2, and LCDR3 regions containing one or two single amino acid substitutions of a VL comprising or contained within the amino acid sequence SEQ ID NO: 10, SEQ ID NO: 12, SEQ ID NO: 14, SEQ ID NO: 16, SEQ ID NO: 17, SEQ ID NO: 19, SEQ ID NO: 21, SEQ ID NO: 23, SEQ ID NO: 24, SEQ ID NO: 26, SEQ ID NO: 28, SEQ ID NO: 29, SEQ ID NO: 31, SEQ ID NO: 33, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 71, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 101, SEQ ID NO: 103, or SEQ ID NO: 110.

In certain aspects the polynucleotide can encode a polypeptide subunit comprising a human IgM or IgA constant region or fragment thereof fused to the C-terminal end of a VH, where the VH comprises an amino acid sequence at least 60%, at least 65%, at least 70%, at least 75%, at least 80%, at least 85%, at least 90%, at least 95% or 100% identical to a mature VH amino acid sequence comprising or contained within any one or more of the amino acid sequences of SEQ ID NO: 9, SEQ ID NO: 11, SEQ ID NO: 13, SEQ ID NO: 15, SEQ ID NO: 18, SEQ ID NO: 20, SEQ ID NO: 22, SEQ ID NO: 25, SEQ ID NO: 27, SEQ ID NO: 30, SEQ ID NO: 32, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 70, SEQ ID NO: 72, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 100, SEQ ID NO: 102, SEQ ID NO: 104, SEQ ID NO: 105, SEQ ID NO: 106, SEQ ID NO: 107, SEQ ID NO: 108, or SEQ ID NO: 109.

In certain aspects the polynucleotide can encode a polypeptide subunit comprising a human light chain constant region or fragment thereof fused to the C-terminal end of a VL, where the VL comprises an amino acid sequence at least at least 60%, at least 65%, at least 70%, at least 75%, at least 80%, at least 85%, at least 90%, at least 95% or 100% identical to a mature VL amino acid sequence comprising or contained within any one or more of SEQ ID NO: 10, SEQ ID NO: 12, SEQ ID NO: 14, SEQ ID NO: 16, SEQ ID NO: 17, SEQ ID NO: 19, SEQ ID NO: 21, SEQ ID NO: 23, SEQ ID NO: 24, SEQ ID NO: 26, SEQ ID NO: 28, SEQ ID NO: 29, SEQ ID NO: 31, SEQ ID NO: 33, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 71, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 101, SEQ ID NO: 103, or SEQ ID NO: 110.

Thus, to form the antigen binding domains, the variable regions of antibodies that specifically bind to GITR can be inserted into expression vector templates for IgM and/or IgA structures, thereby creating multimeric binding molecules having at least two bivalent binding units. In brief, nucleic acid sequences encoding the heavy and light chain variable domain sequences can be synthesized or amplified from existing molecules, and inserted into vectors in the proper orientation and in frame such that upon expression, the vector will yield a full length heavy or light chain. Vectors useful for these purposes are known in the art. Such vectors can also comprise enhancer and other sequences needed to achieve expression of the desired chains. Multiple vectors or single vectors can be used. These vectors are transfected into host cells and then the chains are expressed and purified. Upon expression the chains form fully functional multimeric binding molecules, as has been reported in the literature. The fully assembled multimeric binding molecules can then be purified by standard methods. The expression and purification processes can be performed at commercial scale, if needed.

The disclosure further provides a composition comprising two or more polynucleotides, where the two or more polynucleotides collectively can encode a dimeric, hexameric, or pentameric binding molecule as described above. In certain aspects the composition can include a polynucleotide encoding an IgM and/or IgA heavy chain or fragment thereof, e.g., a human IgM heavy chain as described above where the IgM and/or IgA heavy chain comprises at least the VH of a GITR antigen binding domain, and a polynucleotide encoding a light chain or fragment thereof, e.g., a human kappa or lambda light chain that comprises at least the VL of a GITR antigen binding domain. A polynucleotide composition as provided can further include a polynucleotide encoding a J chain, e.g., a human J chain, or a fragment, variant, or derivative thereof. In certain aspects the polynucleotides making up a composition as provided herein can be situated on two, three, or more separate vectors, e.g., expression vectors. Such vectors are provided by the disclosure. In certain aspects two or more of the polynucleotides making up a composition as provided herein can be situated on a single vector, e.g., an expression vector. Such a vector is provided by the disclosure.

The disclosure further provides a host cell, e.g., a prokaryotic or eukaryotic host cell, comprising a polynucleotide or two or more polynucleotides encoding a dimeric, pentameric, or hexameric GITR agonist binding molecule as provided herein, or any subunit thereof, a polynucleotide composition as provided herein, or a vector or two, three, or more vectors that collectively encode a dimeric, pentameric, or hexameric GITR agonist binding molecule as provided herein, or any subunit thereof. In certain aspects a host cell provided by the disclosure can express a dimeric, pentameric, or hexameric GITR agonist binding molecule as provided by this disclosure, or a subunit thereof.

In a related aspect, the disclosure provides a method of producing a dimeric, pentameric, or hexameric GITR agonist binding molecule as provided by this disclosure, where the method comprises culturing a host cell as described above, and recovering the binding molecule.

Methods of Use

This disclosure provides improved methods for activating signal transduction in cells that express GITR using a dimeric, pentameric, or hexameric IgA- or IgM-based GITR agonist binding molecule as provided herein. The methods described below can utilize binding molecules comprising GITR binding domains derived from any existing GITR antibodies, including without limitation the antibodies provided in Table 2, or variants, derivatives, or analogs thereof. In certain aspects the dimeric, pentameric, or hexameric GITR agonist binding molecule can provide improved activity as compared to an equivalent bivalent antibody, fragment, variant, derivative, or analog in a GITR-expressing cell. For example, upon activation of the receptors by the binding of a dimeric, pentameric, or hexameric binding molecule as provided herein to three or more receptor monomers, the cell, e.g., a T cell, e.g., a Treg or an activated effector CTL, can trigger a signal transduction pathway in the cell and thereby can induce anti-tumor immunity. In certain aspects the use of a dimeric, pentameric, or hexameric GITR agonist binding molecule can result in more potent T cell activation than an equivalent single-binding unit molecule and in turn can induce more potent anti-tumor immunity through, e.g., cytokine release, CTL proliferation, killing of tumor cells, and/or interruption of the suppressive effect of Treg cells in the tumor microenvironment. Based on this disclosure, construction of a dimeric, pentameric, or hexameric IgA- or IgM-based GITR agonist binding molecule comprising any GITR binding domain of interest is well within the capabilities of a person of ordinary skill in the art. The improved activity can, for example, allow a reduced dose to be used, can treat cancers that previously remained untreatable, or can result in more effective or longer-lasting anti-tumor immunity.

In certain aspects, this disclosure provides a method for activating a cell, e.g., a T cell, e.g., a Treg or an activated effector CTL that expresses GITR, where the method includes contacting a GITR-expressing cell with a dimeric, pentameric, or hexameric GITR agonist binding molecule as described herein, where the binding molecule can trigger activation, or enhanced activation, of the GITR-expressing cell. Where the cell is a CTL, “activation” can include, without limitation, increased surface expression of GITR, proliferation, production of proinflammatory cytokines, resistance to the inhibitory effects of CD4+ CD25+ FoxP3+ Treg cells, and/or enhanced killing of tumor cells. Where the cell is a Treg, “activation” can include, without limitation, interference with the cell's ability to suppress anti-tumor immunity in the tumor microenvironment. In certain aspects contacting a GITR-expressing cell with a dimeric, pentameric, or hexameric GITR agonist binding molecule as described herein can induce increased GITR expression, and multimerization of GITR on the cell surface. In certain aspects, contacting a dimeric, pentameric, or hexameric GITR agonist binding molecule as described herein with a GITR-expressing cell, e.g., a T cell, e.g., a Treg or an activated effector CTL that expresses GITR can result in activation of the cell at higher potency than an equivalent amount of a monospecific, bivalent IgG antibody or fragment thereof comprising the same or equivalent GITR binding domains. In certain aspects, contacting a dimeric, pentameric, or hexameric GITR agonist binding molecule as provided herein with a GITR-expressing cell, e.g., a T cell, e.g., a Treg or an activated effector CTL that expresses GITR can result in activation of the cell without the need for secondary cross-linking, e.g., by a FcγR, where an equivalent amount of a monospecific, bivalent IgG antibody or fragment thereof comprising equivalent GITR binding domains would require secondary cross-linking.

In yet another aspect a dimeric, pentameric, or hexameric GITR agonist binding molecule as provided herein can facilitate cancer treatment, e.g., by slowing tumor growth, stalling tumor growth, or reducing the size of existing tumors, when administered as an effective dose to a subject in need of cancer treatment. The disclosure provides a method of treating cancer comprising administering to a subject in need of treatment an effective dose of a dimeric, pentameric, or hexameric GITR agonist binding molecule as provided herein.

The terms “cancer”, “tumor”, “cancerous”, and “malignant” refer to or describe the physiological condition in mammals that is typically characterized by unregulated cell growth. Examples of cancers include but are not limited to, carcinoma including adenocarcinomas, lymphomas, blastomas, melanomas, sarcomas, and leukemias. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer (including hormonally mediated breast cancer, see, e.g., Innes et al. (2006) Br. J. Cancer 94:1057-1065), colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, various types of head and neck cancer including, but not limited to, squamous cell cancers, and cancers of mucinous origins, such as, mucinous ovarian cancer, cholangiocarcinoma (liver) and renal papillary carcinoma.

This disclosure further provides a method of preventing or treating a cancer in a subject in need thereof, comprising administering to the subject an effective amount of a dimeric, pentameric, or hexameric GITR agonist binding molecule as provided herein or a multimeric antigen-binding fragment thereof, a composition or formulation comprising the binding molecule, or a polynucleotide, a vector, or a host cell as described herein.

By “therapeutically effective dose or amount” or “effective amount” is intended an amount of a dimeric, pentameric, or hexameric GITR agonist binding molecule, that when administered brings about a positive immunotherapeutic response with respect to treatment of a cancer patient.

Effective doses of compositions for treatment of cancer vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages can be titrated using routine methods known to those of skill in the art to optimize safety and efficacy.

The subject to be treated can be any animal, e.g., mammal, in need of treatment, in certain aspects, the subject is a human subject.

In its simplest form, a preparation to be administered to a subject is a dimeric, pentameric, or hexameric binding molecule as provided herein, or a multimeric antigen-binding fragment thereof, administered in conventional dosage form, which can be combined with a pharmaceutical excipient, carrier or diluent as described elsewhere herein.

In certain aspects a dimeric, pentameric, or hexameric binding molecule as provided herein may be administered in combination with other cancer therapies, including, but not limited to chemotherapy, radiation therapy, or other immune modulating therapies such as cancer vaccines, immune checkpoint blockade inhibitors, immunostimulatory agents, or adoptive cell transfer such as CAR-T cells.

The compositions of the disclosure can be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In certain aspects, a GITR agonist binding molecule as provided herein or a multimeric antigen-binding fragment thereof can be introduced locally into a tumor, or in the vicinity of a tumor cell, e.g., within the tumor microenvironment (TME).

As noted above, all types of tumors are potentially amenable to treatment by this approach including, without limitation, carcinoma of the breast, lung, pancreas, ovary, kidney, colon and bladder, as well as melanomas, sarcomas and lymphomas. Mucosal distribution could be beneficial for certain cancers, e.g., lung cancer, ovarian cancer, colorectal cancer, or squamous cell carcinoma. A GITR agonist binding molecule as provided herein or a multimeric antigen-binding fragment thereof need not contact the cancer cells or tumor itself to be effective, so it is important to note that the methods of treatment provided herein can be just as effective on cancer cells that do not express GITR as it can be on cancer cells that do express GITR.

A dimeric, pentameric, or hexameric binding molecule for use in the methods provided herein is a binding molecule with two, five, or six binding units as defined herein, that can specifically bind to GITR, e.g., human and/or murine GITR. In certain aspects, a dimeric, pentameric, or hexameric binding molecule for use in the methods provided herein comprises two, five, or six bivalent binding units, respectively, where each binding unit includes two IgA or IgM heavy chain constant regions or fragments thereof. In certain aspects, the two IgA or IgM heavy chain constant regions are human heavy chain constant regions.

Where the binding molecule for use in the methods provided herein is a dimeric IgA-based binding molecule, the binding molecule can further comprise a J chain, or fragment thereof, or variant thereof, and can further comprise a secretory component, or fragment thereof, or variant thereof.

Where the binding molecule for use in the methods provided herein is pentameric IgM-based binding molecule, the binding molecule can further comprise a J chain, or fragment thereof, or variant thereof.

An IgA heavy chain constant region of a binding molecule for use in the methods provided herein can include one or more of a Cα1 domain, a Cα2 domain, and/or a Cα3 domain, provided that the constant region can serve a desired function in the binding molecule, e.g., associate with a light chain constant region to facilitate formation of a binding domain, or associate with another binding unit to form a dimer. In certain aspects the two IgA heavy chain constant regions or fragments thereof within an individual binding unit each comprise a Cα3 domain or fragment thereof, a tailpiece (TP) or fragment thereof, or any combination of a Cα3 domain and a TP or fragment thereof. In certain aspects the two IgA heavy chain constant regions or fragments thereof within an individual binding unit each further comprise a Cα2 domain or fragment thereof, a Cα1 domain or fragment thereof, or a Cα1 domain or fragment thereof and a Cα2 domain or fragment thereof.

An IgM heavy chain constant region of a binding molecule for use in the methods provided herein can include one or more of a Cμ1 domain, a Cμ2 domain, a Cμ3 domain, and/or a Cμ4 domain, provided that the constant region can serve a desired function in the binding molecule, e.g., associate with a light chain constant region to facilitate formation of a binding domain, or associate with other binding units to form a hexamer or a pentamer. In certain aspects the two IgM heavy chain constant regions or fragments thereof within an individual binding unit each comprise a Cμ3 domain or fragment thereof, a Cμ4 domain or fragment thereof, a tailpiece (TP) or fragment thereof, or any combination of a Cμ3 domain a Cμ4 domain, and a TP or fragment thereof. In certain aspects the two IgM heavy chain constant regions or fragments thereof within an individual binding unit each further comprise a Cμ2 domain or fragment thereof, a Cμ1 domain or fragment thereof, or a Cμ1 domain or fragment thereof and a Cμ2 domain or fragment thereof.

While a variety of different dimeric, pentameric, and hexameric binding molecules for use in the methods provided herein can be contemplated by a person of ordinary skill in the art based on this disclosure, and as such are included in this disclosure, in certain aspects, a binding molecule for use in the methods provided herein is provided in which each binding unit comprises two IgA or IgM heavy chains each comprising a VH situated amino terminal to the IgA or IgM constant region or fragment thereof, and two immunoglobulin light chains each comprising a VL situated amino terminal to an immunoglobulin light chain constant region.

Moreover in certain aspects, at least two binding units of the binding molecule for use in the methods provided herein, or at least three, at least four, at least five, or at least six binding units of the binding molecule for use in the methods provided herein, comprise two of the GITR binding domains as described above. In certain aspects the two GITR binding domains in at least two binding units of the binding molecule, or at least three, at least four, at least five, or at least six binding units of the binding molecule for use in the methods provided herein can be different from each other, or they can be identical.

In certain aspects, the two IgA or IgM heavy chains within at least two binding units of the binding molecule, or at least three, at least four, at least five, or at least six binding units of the binding molecule for use in the methods provided herein are identical.

In certain aspects, the two light chains within the at least two binding units of the binding molecule, or at least three, at least four, at least five, or at least six binding units of the binding molecule for use in the methods provided herein are identical. In certain aspects, two identical light chains within at least two binding units, or within at least three, at least four, at least five, or at least six binding units of the binding molecule for use in the methods provided herein are kappa light chains, e.g., human kappa light chains, or lambda light chains, e.g., human lambda light chains.

Dimeric, pentameric, or hexameric GITR agonist binding molecules for use in the methods provided herein can possess advantageous structural or functional properties compared to other binding molecules. For example, a dimeric, pentameric, or hexameric GITR agonist binding molecule for use in the methods provided herein can possess improved activity in a biological assay, either in vitro or in vivo, than a corresponding IgG binding molecule, as describe elsewhere herein.

Pharmaceutical Compositions and Administration Methods

Methods of preparing and administering a dimeric, pentameric, or hexameric GITR agonist binding molecule as provided herein to a subject in need thereof are well known to or are readily determined by those skilled in the art in view of this disclosure. The route of administration of a TNF receptor binding molecule can be, for example, intratumoral, oral, parenteral, by inhalation or topical. The term parenteral as used herein includes, e.g., intravenous, intraarterial, intraperitoneal, intramuscular, subcutaneous, rectal, or vaginal administration. While these forms of administration are contemplated as suitable forms, another example of a form for administration would be a solution for injection, in particular for intratumoral, intravenous, or intraarterial injection or drip. A suitable pharmaceutical composition can comprise a buffer (e.g. acetate, phosphate or citrate buffer), a surfactant (e.g. polysorbate), optionally a stabilizer agent (e.g. human albumin), etc.

As discussed herein, a dimeric, pentameric, or hexameric GITR agonist binding molecule as provided herein can be administered in a pharmaceutically effective amount for the in vivo immunotherapeutic treatment of cancers. In this regard, it will be appreciated that the disclosed binding molecules can be formulated so as to facilitate administration and promote stability of the active agent. Pharmaceutical compositions accordingly can comprise a pharmaceutically acceptable, non-toxic, sterile carrier such as physiological saline, non-toxic buffers, preservatives and the like. A pharmaceutically effective amount of a dimeric, pentameric, or hexameric TNF receptor binding molecule as provided herein means an amount sufficient to achieve effective binding to a target and to achieve a therapeutic benefit. Suitable formulations are described in Remington's Pharmaceutical Sciences (Mack Publishing Co.) 16th ed. (1980).

Certain pharmaceutical compositions provided herein can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of a dimeric, pentameric, or hexameric GITR agonist binding molecule that can be combined with carrier materials to produce a single dosage form will vary depending, e.g., upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, a dimeric, pentameric, or hexameric GITR agonist binding molecule as provided herein can be administered to a subject in need of therapy in an amount sufficient to produce a therapeutic effect. A dimeric, pentameric, or hexameric GITR agonist binding molecule as provided herein can be administered to the subject in a conventional dosage form prepared by combining the antibody or multimeric antigen-binding fragment, variant, or derivative thereof of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

This disclosure also provides for the use of a dimeric, pentameric, or hexameric GITR agonist binding molecule as provided herein in the manufacture of a medicament for treating, preventing, or managing cancer.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

General principles of antibody engineering are set forth in Borrebaeck, ed. (1995) Antibody Engineering (2nd ed.; Oxford Univ. Press). General principles of protein engineering are set forth in Rickwood et al., eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described can be followed as in Current Protocols in Immunology, John Wiley & Sons, New York; Stites et al., eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al., eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al., (Elsevier, Amsterdam); Goldsby et al., eds. (2000) Kuby Immunology (4th ed.; W.H. Freeman and Co., NY); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlag); Sambrook and Russell (2001) Molecular Cloning: A Laboratory Manual (Cold Spring Harbor Press); Lewin (2003) Genes VIII (Prentice Hall, 2003); Harlow and Lane (1988) Antibodies: A Laboratory Manual (Cold Spring Harbor Press); Dieffenbach and Dveksler (2003) PCR Primer (Cold Spring Harbor Press).

All of the references cited above, as well as all references cited herein, are incorporated herein by reference in their entireties.

The following examples are offered by way of illustration and not by way of limitation.

### EXAMPLES

### Example 1: Antibody Generation and Purification

Anti-GITR IgM and Anti-GITR IgG #1 and #2

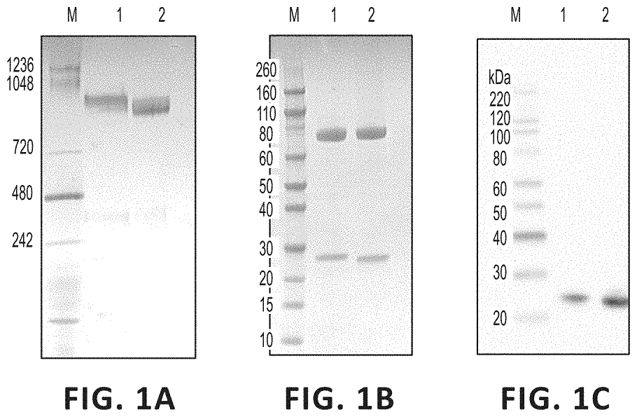

As exemplary constructs, the VH and VL regions of two anti-GITR antibodies from Table 2 were incorporated into IgM (plus wild-type J chain) and IgG formats according to standard cloning protocols. Anti-GITR #1 includes the VH and VL amino acid sequences SEQ ID NO: 49 and SEQ ID NO: 50, respectively, and Anti-GITR #2 includes the VH and VL amino acid sequences SEQ ID NO: 9 and SEQ ID NO: 10, respectively. These antibody constructs were expressed and purified as described below The IgM (plus J-chain) molecule was resolved on reduced and non-reduced gels as follows. FIG. 1A depicts a non-reduced gel to resolve high molecular weight IgMs, and FIG. 1B depicts a reduced gel to show IgM heavy and light chains. For the non-reduced gel, samples were mixed with NUPAGE® LDS Sample Buffer (Life Technologies #NP0007) and loaded onto a NATIVEPAGE™ NOVEX® 3-12% Bis-Tris Gel (Life Technologies #BN1003). NOVEX® Tris-Acetate SDS Running Buffer (Life Technologies #LA0041) was used for gel electrophoresis, and gel was stained with Colloidal Blue Stain (Life Technologies #LC6025). For the reduced gel, samples were mixed with sample buffer and NUPAGE® reducing agent (Life Technologies #NP0004) and heated to 80° C. for 10 minutes and loaded on a NUPAGE® NOVEX® 4-12% Bis-Tris Gel (Life Technologies #NP0322). NUPAGE® MES SDS Running Buffer (Life Technologies #NP0002) was used for gel electrophoresis and gel was stained with Colloidal Blue.

To confirm the presence of the J chain in the IgM pentamer, an anti-J chain western blot was performed (FIG. 1C). For western blotting, proteins were transferred to a membrane using the iBlot system (Life Technologies) according to manufacturer's instructions. Membrane was blocked with 2% BSA in PBS with 0.05% polysorbate-20 sold under the trademark TWEEN®-20, then incubated with anti-J chain antibody (Thermo #MA5-16419) followed by HRP conjugated secondary antibody (Jackson ImmunoResearch #111-035-144) using the iBind system (Life Technologies).

Additional Anti-GITR IgM and IgG Constructs

DTA-1 is a rat anti-mouse GITR monoclonal antibody of the IgG2b isotype, (available, e.g., from eBioscience, Inc. San Diego, Calif.). The VH and VL of DTA-1 are incorporated into rat, mouse, or human IgM and IgG formats according to standard cloning protocols. Anti-human GITR IgMs are generated by incorporating selected VH and VL sequences, e.g., those listed in Table 2, into human IgM and IgG formats according to standard cloning protocols. In addition, new antibodies are generated to human GITR and are selected based on their ability to, e.g., interfere with GITR-GITRL interaction and/or to enable maturation of T cell signaling, T cell proliferation, and/or cytokine secretion. The selected antibody binding domains are reformatted as IgM binding molecules as before.

Protein Expression, Purification and Characterization

Transfection. Heavy, light, and modified or unmodified J chain DNAs (for IgM pentamer constructs) are transfected into, e.g., CHO cells or HEK293 cells. DNA for expression vectors are mixed with polyethylamine (PEI) reagents and then added to cells. PEI transfection with CHO-S cells is conducted according to established techniques (see “Biotechnology and Bioengineering, Vol. 87, 553-545”).

IgG expression products are purified, e.g., using the MabSelectSuRe affinity matrix (GE Life Sciences Catalog #17-5438-01) according to manufacturer's recommendation.

IgM expression products, with or without J chain are purified, e.g., using the Capture Select IgM affinity matrix (BAC, Thermo Fisher Catalog #2890.05) according to manufacturer's recommendation.

### Example 2: Antibody Characterization

Antibody Specificity Measured by ELISA

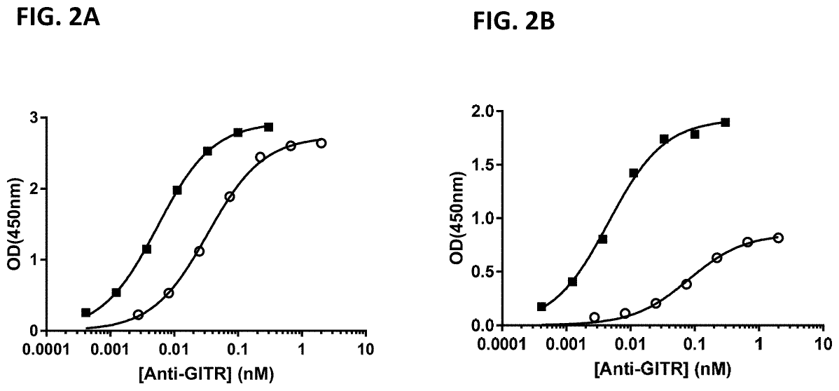

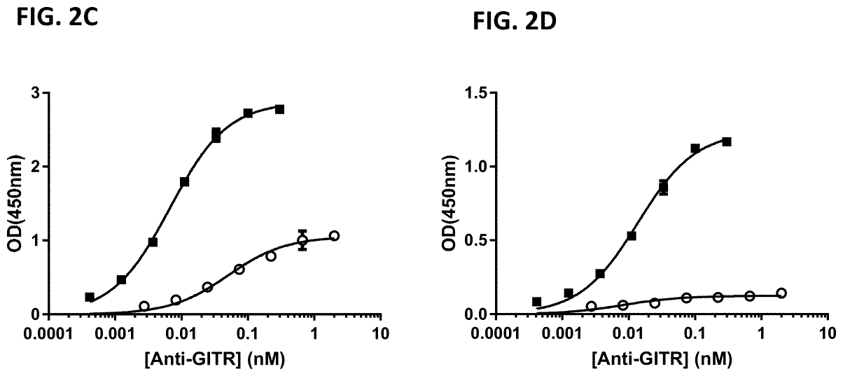

The specificity of the IgG and IgM versions of Anti-GITR #1 and Anti-GITR #2 for human GITR was measured in an ELISA assay at two different antigen densities, as follows. Recombinant human GITR protein (R&D Systems #689-GR-100) was coated onto Maxisorb ELISA plates (Nunc, VWR) in bicarbonate buffer at 10 ng/ml or 1 ng/ml and incubated overnight at 4° C. The plates were washed 3 times with wash buffer (PBS with 0.05% polysorbate-20 sold under the trademark TWEEN®-20) and blocked with blocking buffer (2% BSA in PBS) for 1 hour at room temperature. Serial dilutions of anti-GITR #1 and #2 IgM and IgG antibodies in blocking buffer were added and incubated for 1 hour at room temperature, washed 3 times, then incubated with a 1:6000 dilution of anti-human kappa-HRP (Southern Biotech #9230-05) in blocking buffer for 1 hour at room temperature. Plates were washed 3 times, and then incubated with TMB substrate (BD Biosciences #555214) for 20 minutes at room temperature. The reaction was stopped with 1M H2SO4 and Absorbance at 450 nm was read on a plate reader. The results are shown in FIG. 2A and FIG. 2B (Anti-GITR #1 IgM and IgG at 10 ng/ml and 1 ng/ml antigen densities, respectively), and FIG. 2C and FIG. 2D (Anti-GITR #2 IgM and IgG at 10 ng/ml and 1 ng/ml antigen densities, respectively). All of the constructs specifically bound to human GITR. The results, especially at lower antigen density, show that the IgM constructs bind GITR with much stronger avidity than IgG.

The specificity of chimeric IgG and IgM versions of DTA-1 can be measured in an ELISA assay, e.g., as follows. The extracellular domain of human or mouse GITR is available as his tagged protein (e.g., from Creative Biomart, Shirley, N.Y.). Antigen is coated on plates at a series of decreasing concentrations to determine if multimeric forms of antibodies have an advantage for binding to low antigen density. In this method, 96-well white polystyrene ELISA plates (Pierce 15042) are coated with 100 μL per well of 10 μg/mL or 0.3 μg/mL of his-tagged murine GITR extracellular domain overnight at 4° C. Plates are then washed with 0.05% PBS-polysorbate sold under the trademark TWEEN® and blocked with 2% BSA-PBS. After blocking, 100 μL of serial dilutions of DTA-1-IgM, DTA-1-IgG (or other anti-human antibodies as described above), standards, and controls are added to the wells and incubated at room temperature for 2 hours. The plates are then washed and incubated with HRP conjugated mouse anti-human kappa (Southern Biotech, 9230-05. 1:6000 diluted in 2% BSA-PBS) for 30 min. After 10 final washes using 0.05% PBS-polysorbate sold under the trademark TWEEN®, the plates are read out using SuperSignal chemiluminescent substrate (ThermoFisher, 37070). Luminescent data are collected on an EnVision plate reader (Perkin-Elmer) and analyzed with GraphPad Prism using a 4-parameter logistic model.

Antigen Affinity and Selectivity Measurements

Human or mouse GITR-Ig (Enzo Life Sciences, Inc., Farmingdale, N.Y.), and proteins are plated onto Maxisorb ELISA plates (Nunc, VWR) in bicarbonate buffer at a concentration of 0.2-2.0 μg/ml and incubated overnight at 4° C. Prior to use, plates are thawed, washed once, and then blocked with 0.5% BSA in wash buffer (PBS with 0.05% polysorbate-20 sold under the trademark TWEEN®-20). Various concentrations of anti-GITR MAbs produced as described in Example 1 or control, e.g., isotype-matched anti-KLH antibody are added and samples incubated for 1 h at room temperature, washed 3 times, and incubated with a 1:7,000 dilution of biotinylated anti-human kappa (Southern Biotech, Birmingham, Ala.) in blocking buffer for 1 h. Streptavidin-HRP (Jackson ImmunoResearch, West Grove, Pa.) is then added with TMB substrate (Thermo Scientific, Rockford, Ill.) and the optical density is read on a Spectramax plate reader at 650 nm. Selectivity is calculated as the ratio of the net signal against GITR versus other targets. Further affinity measurements are carried our using a Forte Bio Octet instrument using Biolayer Interferometry (BLI) using immobilized murine or human GITR-Ig. Epitope mapping is assessed against commercially available anti-human GITR antibodies, e.g., 621 (BioLegend), eBioAITR (eBioscience), and MAB689-100 (R&D Systems) as well the GITR ligand (TNFSF18, available from BioLegend).

Testing for GITR Expression

Peripheral blood mononuclear cells (PBMCs) are stained with anti-GITR Mabs produced as described in Example 1 for 30 min at 4° C. Cells are washed, stained with anti-kappa-A647 detection antibody for 15 min at 4° C., and washed again. Binding to CD4+ and CD8+ effector T cells and CD4+ FoxP3+ regulatory T cells is assessed by flow cytometry.

T Cell Binding Assay

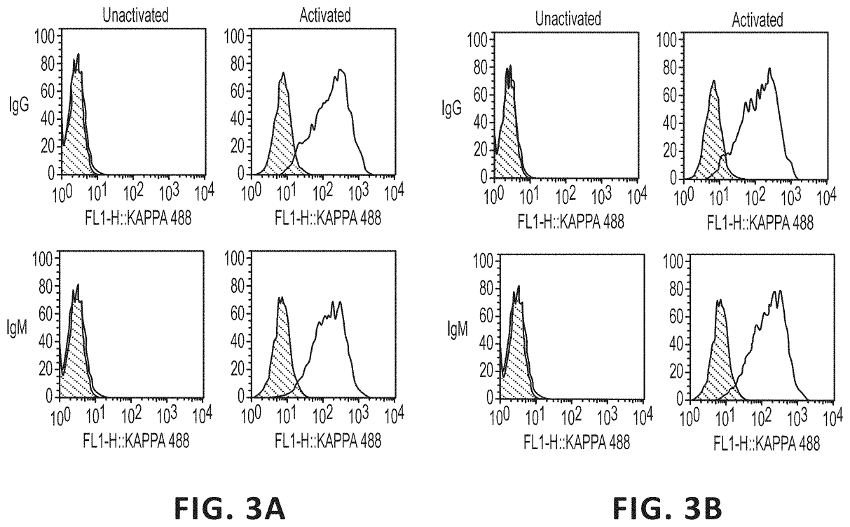

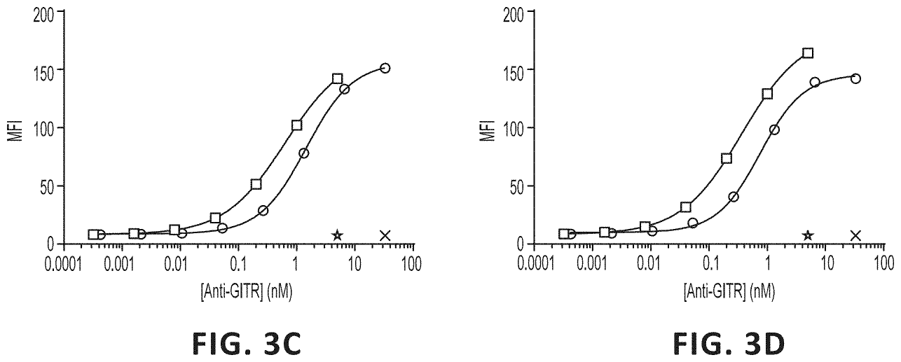

To assess the ability of IgG and IgM antibodies to bind GITR on activated T cells, a binding assay was performed by the following method. Tissue culture plates were coated with 5 μg/mL of anti-CD3 (eBioscience #16-0037-85) at 4° C. overnight, then washed 2 times with PBS. Purified human CD3 T cells (Astarte Biologics) were seeded at 0.1×10⁶ cells/well and incubated with 2 μg/mL of soluble anti-CD28 (Invitrogen #16-0289-85) for 4 days at 37° C. Cells were washed with FACS Stain Buffer (BD Pharmigen Catalog #554656) and 1×10⁵ cells were stained with serial dilutions of anti-GITR antibodies, 5 μg/mL IgG isotype control (Jackson ImmunoResearch #009-000-003), or 5 μg/mL IgM isotype control (Jackson ImmunoResearch #009-000-012) for 30 minutes at 4° C. Cells were washed twice, then stained for 30 minutes at 4° C. with 5 μg/mL anti-human kappa-AF488 secondary antibody (Biolegend #316512), and anti-human CD4-APC (BD Biosciences #555349) and anti-human CD25-PerCP (Biolegend #356111) for immunophenotyping. Cells were washed twice, resuspended in FACS Stain Buffer, and acquired by flow cytometry. The results are shown in FIG. 3A (Anti-GITR IgG and IgM #1) and FIG. 3B (Anti-GITR IgG and IgM #2). Filled histograms, Isotype controls; Open histograms, Anti-GITR antibodies. All the constructs bound to the activated T cells.

IgG and IgM binding dose response on activated T cells was also plotted. The results are shown in FIG. 3C (Anti-GITR IgG and IgM #1) and FIG. 3D (Anti-GITR IgG and IgM #2). The results of these binding assays demonstrate that the antibody constructs bind GITR on activated T cells.

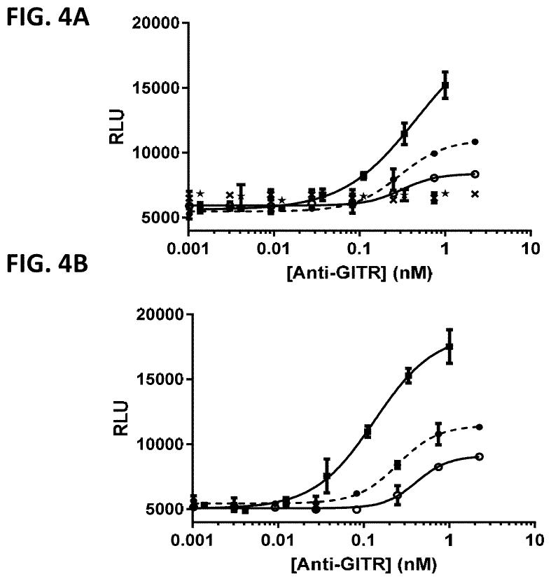

Agonist activity of antibodies was determined using the GITR Bioassay (Promega #CS184006), a NFκB reporter assay. The assay was performed according to manufacturer's protocol. NFκB-luc2/GITR Jurkat cells were incubated with serial dilutions of anti-GITR #1 and #2 IgM, anti-GITR #1 and #2 IgG alone and also with 10 μg/mL plate-bound anti-human IgG Fc crosslinker (Biolegend #409302), IgG isotype control (Jackson ImmunoResearch #009-000-003), or IgM isotype control (Jackson ImmunoResearch #009-000-012) for 6 hours at 37° C. Bio-Glo reagent was added and after 10 minutes luminescence was read on a plate reader. The results for Anti-GITR #1 are shown in FIG. 4A and the results for Anti-GITR #2 are shown in FIG. 4B. For two different Anti-GITR antibodies, IgM was a stronger inducer of GITR signaling than both crosslinked and uncrosslinked IgG, demonstrating the ability of the IgM constructs to act as a superagonist.

T Cell Proliferation Assay

To test for T cell proliferation, Anti-GITR Mabs produced as described in Example 1 are coated on a plate with or without anti-CD3 Mab for 1 hour, and then naïve T cells are plated. After 15 hours, T cell proliferation is measured using the Cell Titer Glo luminescent reagent (Promega). To evaluate effector T cell proliferation in the presence of regulatory T cells, effector T cells are labeled with carboxyfluorescein succinimidyl ester (CFSE) dye, mixed with regulatory T cells at a 1:1; ratio, then added to a plate pre-coated with anti-GITR Mab with or without anti-CD3. Effector T cell proliferation is monitored by flow cytometry.

T Cell Activation and Cytokine Secretion

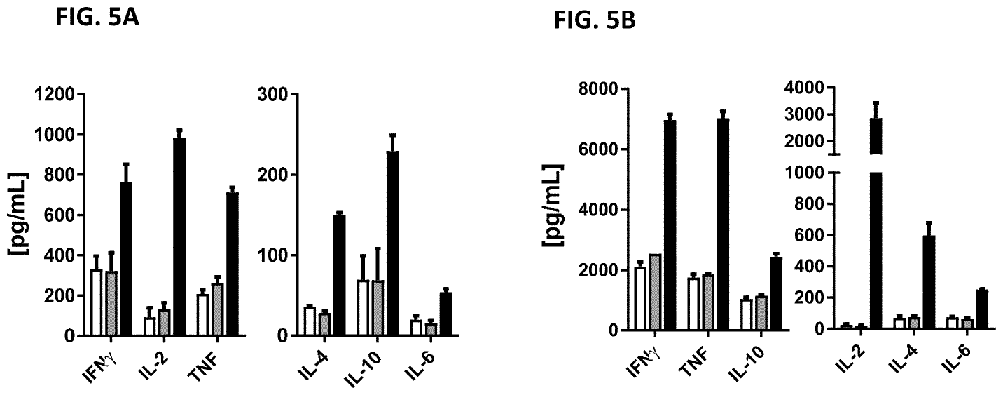

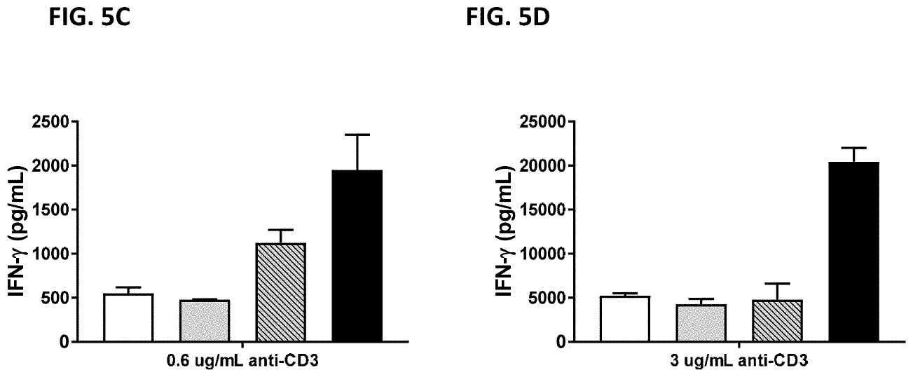

The ability of the Anti-GITR #1 IgM construct to enhance T cell activation was assessed as follows. 96-well tissue culture plates were coated with a suboptimal (0.6 μg/mL) and a high (3 μg/mL) dose of anti-CD3 (clone OKT3, eBioscience #16-0037-85) at 4° C. overnight, and then washed 2 times with PBS. For IgG crosslinking, wells were additionally coated with 10 μg/mL of anti-human IgG Fc crosslinker (Biolegend #409302). Purified human CD4 T cells (Astarte Biologics) were seeded at 0.2×10⁶ cells/well and incubated with 1 μg/mL of soluble anti-CD28 (Invitrogen #16-0289-85) and 10 μg/mL of soluble anti-GITR #1 IgM or IgG antibodies for 4 days at 37° C. Supernatants were assayed for a panel of cytokines including IFNγ, IL-4, TNF, IL-10, and IL-6 by cytometric bead array (CBA) according to manufacturer's protocol (BD #551809). The results, shown in FIG. 5A-B, demonstrate that Anti-GITR #1 IgM enhanced T cell activation. At both suboptimal (FIG. 5A) and high (FIG. 5B) concentrations of anti-CD3, anti-GITR IgM induced more IFNγ, IL-2, IL-4, TNF, IL-10, and IL-6 production than anti-GITR IgG.

The effect of IgG cross-linking was also assessed. Supernatants produced as above were collected and IFNγ was quantitated by ELISA according to manufacturer's protocol (R&D Systems #DY285B). At both suboptimal (FIG. 5C) and high (FIG. 5D) concentrations of anti-CD3, anti-GITR IgM #1 demonstrates enhanced IFNγ production than cross-linked anti-GITR IgG.

In an alternative assay, T cells are stimulated with anti-GITR Mabs produced as described in Example 1 in the presence or absence of anti-CD3 antibody. After 24 hours, IFNγ+ and TNFα+ T cells are analyzed by flow cytometry. Additionally, cytokines IL-2 and IFNγ secreted in the supernatant are measured using a standard ELISA kit.

T Cell Mediated Cytotoxicity

Effector T cells are stimulated with tumor cell specific peptide for 7 days. Murine CT26 or MC38 colon tumor cells or B16-F10 melanoma cells are labeled with CFSE dye, then mixed with activated T cells and anti-GITR Mabs produced as described in Example 1. After 24 hours, tumor cell cytotoxicity is measured by flow cytometry.

In Vivo Activity

For DTA 1-IgM and DTA-1 IgG antibodies, syngeneic mouse models are used. Balb/c mice are implanted with CT26, MC38, or B16-F10 tumor cells subcutaneously, and then mice are randomized according to tumor size. Animals are then dosed with DTA-1 IgG, DTA-1 IgM, or vehicle control and tumor volume is measured.

For anti-human GITR Mabs produced as described in Example 1, GITR knock-in HuGEMM mouse models are used (Crown Bio). Murine GITR is knocked out and replaced with human GITR in the mouse model. CT26, MC38, or B16-F10 tumors are implanted subcutaneously, mice are dosed with anti-GITR IgG or IgM or vehicle, and tumor volume is measured.

What is claimed is:

1. A multimeric binding molecule comprising two, five, or six bivalent binding units or variants or fragments thereof,
wherein each binding unit comprises two IgA or IgM heavy chain constant regions or fragments thereof, each associated with an antigen-binding domain,
wherein at least three of the antigen-binding domains of the binding molecule can specifically and agonistically bind to a GITR monomer on a cell expressing GITR, and
wherein the binding molecule can induce GITR-mediated signal transduction in the cell in the absence of a secondary cross-linking moiety.

2. The multimeric binding molecule of claim 1, which can bind to and engage three or more GITR monomers expressed on the surface of the cell in the absence of a secondary cross-linking moiety

3. The multimeric binding molecule of claim 1 or claim 2, wherein the cell expressing GITR is a T cell.

4. The multimeric binding molecule of claim 3, wherein the T cell is a cytotoxic T lymphocyte (CTL).

5. The multimeric binding molecule of claim 3 or claim 4, wherein GITR-mediated signal transduction in the cell can increase surface expression of GITR, increase CTL proliferation, increase production of proinflammatory cytokines, increase resistance to the inhibitory effects of CD4+ CD25+ FoxP3+ Treg cells, increase or enhance killing of tumor cells, or a combination thereof.

6. The multimeric binding molecule of claim 3, wherein the T cell is a CD4+ CD25+ FoxP3+ Treg cell.

7. The multimeric binding molecule of claim 3 or claim 6, wherein GITR-mediated signal transduction in the cell can interference with the cell's ability to suppress anti-tumor immunity in the tumor microenvironment.

8. The multimeric binding molecule of any one of claims 1 to 7, which can induce GITR-mediated signal transduction in the cell expressing GITR at a higher potency than an equivalent amount of a bivalent IgG antibody or fragment thereof comprising two equivalent GITR antigen-binding domains.

9. The multimeric binding molecule of any one of claims 1 to 8, which comprises at least three, at least four, at least five, at least six, at least seven, at least eight, at least nine, at least ten, at least eleven, or twelve antigen-binding domains that specifically and agonistically bind to a GITR monomer expressed on the surface of the cell, thereby activating GITR-mediated signal transduction in the cell.

10. The multimeric binding molecule of claim 9, wherein the at least three, at least four, at least five, at least six, at least seven, at least eight, at least nine, at least ten, at least eleven, or twelve antigen-binding domains bind to the same extracellular GITR epitope.

11. The multimeric binding molecule of claim 9, wherein at least three, at least four, at least five, at least six, at least seven, at least eight, at least nine, at least ten, at least eleven, or twelve antigen-binding domains each specifically bind one of a group of two or more different extracellular GITR epitopes.

12. The multimeric binding molecule of any one of claims 1 to 11, wherein the two, five, or six binding units are human, humanized, or chimeric immunoglobulin binding units.

13. The multimeric binding molecule of any one of claims 1 to 12, wherein at the least three antigen-binding domains of the binding molecule are GITR agonist binding domains, and wherein at least one, at least two, at least three, at least four, at least five, at least six, at least seven, at least eight, at least nine, at least ten, at least eleven, or twelve antigen-binding domains comprise a heavy chain variable region (VH) and a light chain variable region (VL), wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL amino acid sequences comprising or contained within SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 15 and SEQ ID NO: 17; SEQ ID NO: 18 and SEQ ID NO: 19; SEQ ID NO: 20 and SEQ ID NO: 21; SEQ ID NO: 22 and SEQ ID NO: 23; SEQ ID NO: 22 and SEQ ID NO: 24; SEQ ID NO: 25 and SEQ ID NO: 26; SEQ ID NO: 27 and SEQ ID NO: 28; SEQ ID NO: 27 and SEQ ID NO: 29; SEQ ID NO: 30 and SEQ ID NO: 31; SEQ ID NO: 32 and SEQ ID NO: 33; SEQ ID NO: 32 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 57 and SEQ ID NO: 58; SEQ ID NO: 59 and SEQ ID NO: 60; SEQ ID NO: 61 and SEQ ID NO: 62; SEQ ID NO: 63 and SEQ ID NO: 64; SEQ ID NO: 65 and SEQ ID NO: 66; SEQ ID NO: 67 and SEQ ID NO: 68; SEQ ID NO: 69 and SEQ ID NO: 68; SEQ ID NO: 70 and SEQ ID NO: 71; SEQ ID NO: 72 and SEQ ID NO: 71; SEQ ID NO: 73 and SEQ ID NO: 74; SEQ ID NO: 75 and SEQ ID NO: 76; SEQ ID NO: 77 and SEQ ID NO: 78; SEQ ID NO: 79 and SEQ ID NO: 80; SEQ ID NO: 81 and SEQ ID NO: 82; SEQ ID NO: 83 and SEQ ID NO: 84; SEQ ID NO: 85 and SEQ ID NO: 86; SEQ ID NO: 87 and SEQ ID NO: 88; SEQ ID NO: 89 and SEQ ID NO: 90; SEQ ID NO: 91 and SEQ ID NO: 92; SEQ ID NO: 93 and SEQ ID NO: 94; SEQ ID NO: 95 and SEQ ID NO: 96; SEQ ID NO: 97 and SEQ ID NO: 98; SEQ ID NO: 99 and SEQ ID NO: 98; SEQ ID NO: 100 and SEQ ID NO: 101; SEQ ID NO: 102 and SEQ ID NO: 103; SEQ ID NO: 104 and SEQ ID NO: 101; SEQ ID NO: 105 and SEQ ID NO: 101; SEQ ID NO: 106 and SEQ ID NO: 101; SEQ ID NO: 107 and SEQ ID NO: 101; SEQ ID NO: 108 and SEQ ID NO: 101; or SEQ ID NO: 109 and SEQ ID NO: 110, respectively or the CDRs of an antibody comprising the VH and VL amino acid sequences comprising or contained within SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 15 and SEQ ID NO: 17; SEQ ID NO: 18 and SEQ ID NO: 19; SEQ ID NO: 20 and SEQ ID NO: 21; SEQ ID NO: 22 and SEQ ID NO: 23; SEQ ID NO: 22 and SEQ ID NO: 24; SEQ ID NO: 25 and SEQ ID NO: 26; SEQ ID NO: 27 and SEQ ID NO: 28; SEQ ID NO: 27 and SEQ ID NO: 29; SEQ ID NO: 30 and SEQ ID NO: 31; SEQ ID NO: 32 and SEQ ID NO: 33; SEQ ID NO: 32 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 57 and SEQ ID NO: 58; SEQ ID NO: 59 and SEQ ID NO: 60; SEQ ID NO: 61 and SEQ ID NO: 62; SEQ ID NO: 63 and SEQ ID NO: 64; SEQ ID NO: 65 and SEQ ID NO: 66; SEQ ID NO: 67 and SEQ ID NO: 68; SEQ ID NO: 69 and SEQ ID NO: 68; SEQ ID NO: 70 and SEQ ID NO: 71; SEQ ID NO: 72 and SEQ ID NO: 71; SEQ ID NO: 73 and SEQ ID NO: 74; SEQ ID NO: 75 and SEQ ID NO: 76; SEQ ID NO: 77 and SEQ ID NO: 78; SEQ ID NO: 79 and SEQ ID NO: 80; SEQ ID NO: 81 and SEQ ID NO: 82; SEQ ID NO: 83 and SEQ ID NO: 84; SEQ ID NO: 85 and SEQ ID NO: 86; SEQ ID NO: 87 and SEQ ID NO: 88; SEQ ID NO: 89 and SEQ ID NO: 90; SEQ ID NO: 91 and SEQ ID NO: 92; SEQ ID NO: 93 and SEQ ID NO: 94; SEQ ID NO: 95 and SEQ ID NO: 96; SEQ ID NO: 97 and SEQ ID NO: 98; SEQ ID NO: 99 and SEQ ID NO: 98; SEQ ID NO: 100 and SEQ ID NO: 101; SEQ ID NO: 102 and SEQ ID NO: 103; SEQ ID NO: 104 and SEQ ID NO: 101; SEQ ID NO: 105 and SEQ ID NO: 101; SEQ ID NO: 106 and SEQ ID NO: 101; SEQ ID NO: 107 and SEQ ID NO: 101; SEQ ID NO: 108 and SEQ ID NO: 101; or SEQ ID NO: 109 and SEQ ID NO: 110, respectively, except for one or two amino acid substitutions in one or more of the CDRs.

14. The multimeric binding molecule of any one of claims 1 to 13, wherein at least one, at least two, at least three, at least four, at least five, at least six, at least seven, at least eight, at least nine, at least ten, at least eleven, or twelve antigen-binding domains comprise an antibody VH and a VL, wherein the VH and VL comprise amino acid sequences at least 80%, at least 85%, at least 90%, at least 95% or 100% identical to the mature VH and VL amino acid sequences comprising or contained within SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 15 and SEQ ID NO: 17; SEQ ID NO: 18 and SEQ ID NO: 19; SEQ ID NO: 20 and SEQ ID NO: 21; SEQ ID NO: 22 and SEQ ID NO: 23; SEQ ID NO: 22 and SEQ ID NO: 24; SEQ ID NO: 25 and SEQ ID NO: 26; SEQ ID NO: 27 and SEQ ID NO: 28; SEQ ID NO: 27 and SEQ ID NO: 29; SEQ ID NO: 30 and SEQ ID NO: 31; SEQ ID NO: 32 and SEQ ID NO: 33; SEQ ID NO: 32 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 57 and SEQ ID NO: 58; SEQ ID NO: 59 and SEQ ID NO: 60; SEQ ID NO: 61 and SEQ ID NO: 62; SEQ ID NO: 63 and SEQ ID NO: 64; SEQ ID NO: 65 and SEQ ID NO: 66; SEQ ID NO: 67 and SEQ ID NO: 68; SEQ ID NO: 69 and SEQ ID NO: 68; SEQ ID NO: 70 and SEQ ID NO: 71; SEQ ID NO: 72 and SEQ ID NO: 71; SEQ ID NO: 73 and SEQ ID NO: 74; SEQ ID NO: 75 and SEQ ID NO: 76; SEQ ID NO: 77 and SEQ ID NO: 78; SEQ ID NO: 79 and SEQ ID NO: 80; SEQ ID NO: 81 and SEQ ID NO: 82; SEQ ID NO: 83 and SEQ ID NO: 84; SEQ ID NO: 85 and SEQ ID NO: 86; SEQ ID NO: 87 and SEQ ID NO: 88; SEQ ID NO: 89 and SEQ ID NO: 90; SEQ ID NO: 91 and SEQ ID NO: 92; SEQ ID NO: 93 and SEQ ID NO: 94; SEQ ID NO: 95 and SEQ ID NO: 96; SEQ ID NO: 97 and SEQ ID NO: 98; SEQ ID NO: 99 and SEQ ID NO: 98; SEQ ID NO: 100 and SEQ ID NO: 101; SEQ ID NO: 102 and SEQ ID NO: 103; SEQ ID NO: 104 and SEQ ID NO: 101; SEQ ID NO: 105 and SEQ ID NO: 101; SEQ ID NO: 106 and SEQ ID NO: 101; SEQ ID NO: 107 and SEQ ID NO: 101; SEQ ID NO: 108 and SEQ ID NO: 101; or SEQ ID NO: 109 and SEQ ID NO: 110, respectively.

15. The multimeric binding molecule of any one of claims 1 to 14, which is a dimeric binding molecule comprising two bivalent IgA binding units or fragments thereof and a J chain or fragment or variant thereof, wherein each binding unit comprises two IgA heavy chain constant regions or fragments thereof each associated with an antigen-binding domain.

16. The multimeric binding molecule of claim 15, further comprising a secretory component, or fragment or variant thereof.

17. The multimeric binding molecule of claim 15 or claim 16, wherein the IgA heavy chain constant regions or fragments thereof each comprise a Cα2 domain or a Cα3-tp domain.

18. The multimeric binding molecule of claim 17, wherein one or more IgA heavy chain constant regions or fragments thereof further comprise a Cα1 domain.

19. The multimeric binding molecule of any one of claims 15 to 18, wherein the IgA heavy chain constant region is a human IgA constant region.

20. The multimeric binding molecule of any one of claims 15 to 19, wherein each binding unit comprises two IgA heavy chains each comprising a VH situated amino terminal to the IgA constant region or fragment thereof, and two immunoglobulin light chains each comprising a VL situated amino terminal to an immunoglobulin light chain constant region.

21. The multimeric binding molecule of any one of claims 1 to 14, which is a pentameric or a hexameric binding molecule comprising five or six bivalent IgM binding units, respectively, wherein each binding unit comprises two IgM heavy chain constant regions or fragments thereof each associated with an antigen-binding domain.

22. The multimeric binding molecule of claim 21, wherein the IgM heavy chain constant regions or fragments thereof each comprise a Cμ3 domain or fragment or variant thereof and a Cμ4-tp domain or fragment or variant thereof.

23. The multimeric binding molecule of claim 21 or claim 22, wherein one or more IgM heavy chain constant regions or fragments thereof further comprise a Cμ2 domain, a Cμ1 domain, or any combination thereof.

24. The multimeric binding molecule of any one of claims 21 to 23, wherein the binding molecule is pentameric, and further comprises a J chain, or fragment thereof, or variant thereof.

25. The multimeric binding molecule of any one of claims 21 to 24, wherein the IgM heavy chain constant region is a human IgM constant region.

26. The multimeric binding molecule of any one of claims 21 to 25, wherein each binding unit comprises two IgM heavy chains each comprising a VH situated amino terminal to the IgM constant region or fragment thereof, and two immunoglobulin light chains each comprising a VL situated amino terminal to an immunoglobulin light chain constant region.

27. The multimeric binding molecule of any one of claims 1 to 26, wherein each binding unit comprises two heavy chains and two light chains, wherein the heavy chains and light chains comprise VH and VL amino acid sequences at least 80%, at least 85%, at least 90%, at least 95% or 100% identical to the mature VH and VL amino acid sequences comprising or contained within SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 15 and SEQ ID NO: 17; SEQ ID NO: 18 and SEQ ID NO: 19; SEQ ID NO: 20 and SEQ ID NO: 21; SEQ ID NO: 22 and SEQ ID NO: 23; SEQ ID NO: 22 and SEQ ID NO: 24; SEQ ID NO: 25 and SEQ ID NO: 26; SEQ ID NO: 27 and SEQ ID NO: 28; SEQ ID NO: 27 and SEQ ID NO: 29; SEQ ID NO: 30 and SEQ ID NO: 31; SEQ ID NO: 32 and SEQ ID NO: 33; SEQ ID NO: 32 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 57 and SEQ ID NO: 58; SEQ ID NO: 59 and SEQ ID NO: 60; SEQ ID NO: 61 and SEQ ID NO: 62; SEQ ID NO: 63 and SEQ ID NO: 64; SEQ ID NO: 65 and SEQ ID NO: 66; SEQ ID NO: 67 and SEQ ID NO: 68; SEQ ID NO: 69 and SEQ ID NO: 68; SEQ ID NO: 70 and SEQ ID NO: 71; SEQ ID NO: 72 and SEQ ID NO: 71; SEQ ID NO: 73 and SEQ ID NO: 74; SEQ ID NO: 75 and SEQ ID NO: 76; SEQ ID NO: 77 and SEQ ID NO: 78; SEQ ID NO: 79 and SEQ ID NO: 80; SEQ ID NO: 81 and SEQ ID NO: 82; SEQ ID NO: 83 and SEQ ID NO: 84; SEQ ID NO: 85 and SEQ ID NO: 86; SEQ ID NO: 87 and SEQ ID NO: 88; SEQ ID NO: 89 and SEQ ID NO: 90; SEQ ID NO: 91 and SEQ ID NO: 92; SEQ ID NO: 93 and SEQ ID NO: 94; SEQ ID NO: 95 and SEQ ID NO: 96; SEQ ID NO: 97 and SEQ ID NO: 98; SEQ ID NO: 99 and SEQ ID NO: 98; SEQ ID NO: 100 and SEQ ID NO: 101; SEQ ID NO: 102 and SEQ ID NO: 103; SEQ ID NO: 104 and SEQ ID NO: 101; SEQ ID NO: 105 and SEQ ID NO: 101; SEQ ID NO: 106 and SEQ ID NO: 101; SEQ ID NO: 107 and SEQ ID NO: 101; SEQ ID NO: 108 and SEQ ID NO: 101; or SEQ ID NO: 109 and SEQ ID NO: 110, respectively.

28. The multimeric binding molecule of any one of claims 1 to 14 or 21 to 27, wherein the binding molecule is a pentameric IgM molecule, further comprising a J chain or fragment or variant thereof.

29. A composition comprising the multimeric binding molecule of any one of claims 1 to 28.

30. A polynucleotide comprising a nucleic acid sequence that encodes a polypeptide subunit of the binding molecule of any one of claims 1 to 28.

31. The polynucleotide of claim 30, wherein the polypeptide subunit comprises an IgM heavy chain constant region and at least an antibody VH portion of the antigen-binding domain of the multimeric binding molecule.

32. The polynucleotide of claim 31, wherein the polypeptide subunit comprises a human IgM constant region or fragment thereof fused to the C-terminal end of a VH comprising:
(a) HCDR1, HCDR2, and HCDR3 regions comprising the CDRs contained in the VH amino acid sequence comprising or contained within SEQ ID NO: 9, SEQ ID NO: 11, SEQ ID NO: 13, SEQ ID NO: 15, SEQ ID NO: 18, SEQ ID NO: 20, SEQ ID NO: 22, SEQ ID NO: 25, SEQ ID NO: 27, SEQ ID NO: 30, SEQ ID NO: 32, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 70, SEQ ID NO: 72, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 100, SEQ ID NO: 102, SEQ ID NO: 104, SEQ ID NO: 105, SEQ ID NO: 106, SEQ ID NO: 107, SEQ ID NO: 108, or SEQ ID NO: 109; or the CDRs contained in the VH amino acid sequence comprising or contained within SEQ ID NO: 9, SEQ ID NO: 11, SEQ ID NO: 13, SEQ ID NO: 15, SEQ ID NO: 18, SEQ ID NO: 20, SEQ ID NO: 22, SEQ ID NO: 25, SEQ ID NO: 27, SEQ ID NO: 30, SEQ ID NO: 32, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 70, SEQ ID NO: 72, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 100, SEQ ID NO: 102, SEQ ID NO: 104, SEQ ID NO: 105, SEQ ID NO: 106, SEQ ID NO: 107, SEQ ID NO: 108, or SEQ ID NO: 109 with one or two single amino acid substitutions in one or more of the HCDRs; or
(b) an amino acid sequence at least 80%, at least 85%, at least 90%, at least 95% or 100% identical to the mature VH amino acid sequence comprising or contained within SEQ ID NO: 9, SEQ ID NO: 11, SEQ ID NO: 13, SEQ ID NO: 15, SEQ ID NO: 18, SEQ ID NO: 20, SEQ ID NO: 22, SEQ ID NO: 25, SEQ ID NO: 27, SEQ ID NO: 30, SEQ ID NO: 32, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 70, SEQ ID NO: 72, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 100, SEQ ID NO: 102, SEQ ID NO: 104, SEQ ID NO: 105, SEQ ID NO: 106, SEQ ID NO: 107, SEQ ID NO: 108, or SEQ ID NO: 109.

33. The polynucleotide of any one of claims 30 to 32, wherein the polypeptide subunit comprises a light chain constant region and an antibody VL portion of the antigen-binding domain of the multimeric binding molecule.

34. The polynucleotide of claim 33, wherein the polypeptide subunit comprises a human kappa or lambda light chain constant region or fragment thereof fused to the C-terminal end of a VL comprising:
(a) LCDR1, LCDR2, and LCDR3 regions comprising the CDRs contained in the VL amino acid sequence comprising or contained within SEQ ID NO: 10, SEQ ID NO: 12, SEQ ID NO: 14, SEQ ID NO: 16, SEQ ID NO: 17, SEQ ID NO: 19, SEQ ID NO: 21, SEQ ID NO: 23, SEQ ID NO: 24, SEQ ID NO: 26, SEQ ID NO: 28, SEQ ID NO: 29, SEQ ID NO: 31, SEQ ID NO: 33, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 71, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 101, SEQ ID NO: 103, or SEQ ID NO: 110; or the CDRs contained in the VL amino acid sequence SEQ ID NO: 10, SEQ ID NO: 12, SEQ ID NO: 14, SEQ ID NO: 16, SEQ ID NO: 17, SEQ ID NO: 19, SEQ ID NO: 21, SEQ ID NO: 23, SEQ ID NO: 24, SEQ ID NO: 26, SEQ ID NO: 28, SEQ ID NO: 29, SEQ ID NO: 31, SEQ ID NO: 33, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 71, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 101, SEQ ID NO: 103, or SEQ ID NO: 110 with one or two single amino acid substitutions in one or more of the LCDRs; or
(b) an amino acid sequence at least 80%, at least 85%, at least 90%, at least 95% or 100% identical to the mature VL amino acid sequence comprising or contained within SEQ ID NO: 10, SEQ ID NO: 12, SEQ ID NO: 14, SEQ ID NO: 16, SEQ ID NO: 17, SEQ ID NO: 19, SEQ ID NO: 21, SEQ ID NO: 23, SEQ ID NO: 24, SEQ ID NO: 26, SEQ ID NO: 28, SEQ ID NO: 29, SEQ ID NO: 31, SEQ ID NO: 33, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 71, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 101, SEQ ID NO: 103, or SEQ ID NO: 110.

35. A composition comprising the polynucleotide of any one of claims 30 to 32, and the polynucleotide of any one of claim 30, 33, or 34.

36. The composition of claim 35, wherein the polynucleotides are on separate vectors.

37. The composition of claim 35, wherein the polynucleotides are on a single vector.

38. The composition of any one of claims 35 to 37, further comprising a polynucleotide comprising a nucleic acid sequence encoding a J chain, or fragment thereof, or variant thereof.

39. The vector of claim 37.

40. The vectors of claim 36.

41. A host cell comprising the polynucleotide of any one of claims 30 to 34, the composition of any one of claims 35 to 38, or the vector or vectors of any one of claim 39 or 40, wherein the host cell can express the binding molecule of any one of claims 1 to 28, or a subunit thereof.

42. A method of producing the binding molecule of any one of claims 1 to 28, comprising culturing the host cell of claim 41, and recovering the binding molecule.

43. A method of inducing GITR-mediated activation in a GITR-expressing cell, comprising contacting the GITR-expressing cell with the multimeric binding molecule of any one of claims 1 to 28.

44. A method of inducing GITR translocation and clustering in GITR-expressing T cells, comprising contacting GITR-expressing T cells with the multimeric binding molecule of any one of claims 1 to 28.

45. A method of treating cancer comprising administering to a subject in need of treatment an effective amount of the multimeric binding molecule of any one of claims 1 to 28, wherein the multimeric binding molecule can activate GITR-expressing CTL cells thereby triggering a tumoricidal CTL response.

46. The method of claim 45, wherein the subject is human.
